(19) United States
(12) Patent Application Publication
(10) Pub. No.: US 2010/0069347 A1
(43) Pub. Date: Mar. 18, 2010

(54) HETEROCYCLYL-SUBSTITUTED ANTI-HYPERCHOLESTEROLEMIC COMPOUNDS

(76) Inventors: Gregori J. Morriello, Westfield, NJ (US); Robert J. DeVita, Westfield, NJ (US); Christopher R. Moyes, Westfield, NJ (US)

Correspondence Address: MERCK AND CO., INC, P O BOX 2000, RAHWAY, NJ, 07065-0907, US

(21) Appl. No.: 12/513,010

(22) PCT Filed: Oct. 30, 2007
(86) PCT No.: PCT/US07/22895

§ 371 Date: Apr. 30, 2009

Related U.S. Application Data
(60) Provisional application No. 60/856,213, filed on Nov. 2, 2006.
(60) Provisional application No. 60/931,236, filed on May 22, 2007.

(51) Int. Cl. A61K 31/397; C07D 205/08; A61P 9/10

(52) U.S. Cl. 514/210.02; 540/200

(57) ABSTRACT

This invention provides cholesterol absorption inhibitors of Formula I: and the pharmaceutically acceptable salts thereof, wherein R¹² is a hydroxylated alkyl group and R⁹ contains a heterocyclic ring. The compounds are useful for lowering plasma cholesterol levels, particularly LDL cholesterol, and for treating atherosclerosis and preventing atherosclerotic disease events.

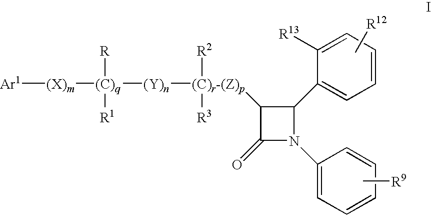

### BACKGROUND OF THE INVENTION

The instant invention relates to substituted 2-azetidinones and the pharmaceutically acceptable salts and esters there of, and to their use alone or in combination with other active agents to treat hypercholesterolemia and for preventing, halting or slowing the progression of atherosclerosis and related conditions and disease events.

It has been clear for several decades that elevated blood cholesterol is a major risk factor for coronary heart disease, and many studies have shown that the risk of CHD events can be reduced by lipid-lowering therapy. Prior to 1987, the lipid-lowering armamentarium was limited essentially to a low saturated fat and cholesterol diet, the bile acid sequestrants (cholestyramine and colestipol), nicotinic acid (niacin), the fibrates and probucol. Unfortunately, all of these treatments have limited efficacy or tolerability, or both. Substantial reductions in LDL (low density lipoprotein) cholesterol accompanied by increases in HDL (high density lipoprotein) cholesterol could be achieved by the combination of a lipid-lowering diet and a bile acid sequestrant, with or without the addition of nicotinic acid. However, this therapy is not easy to administer or tolerate and was therefore often unsuccessful except in specialist lipid clinics. The fibrates produce a moderate reduction in LDL cholesterol accompanied by increased HDL cholesterol and a substantial reduction in triglycerides, and because they are well tolerated these drugs have been more widely used. Probucol produces only a small reduction in LDL cholesterol and also reduces HDL cholesterol, which, because of the strong inverse relationship between HDL cholesterol level and CHD risk, is generally considered undesirable. With the introduction of lovastatin, the first inhibitor of HMG-CoA reductase to become available for prescription in 1987, for the first time physicians were able to obtain large reductions in plasma cholesterol with very few adverse effects.

Studies have unequivocally demonstrated that lovastatin, simvastatin and pravastatin, all members of the HMG-CoA reductase inhibitor class, slow the progression of atherosclerotic lesions in the coronary and carotid arteries. Simvastatin and pravastatin have also been shown to reduce the risk of coronary heart disease events, and in the case of simvastatin a highly significant reduction in the risk of coronary death and total mortality has been shown by the Scandinavian Simvastatin Survival Study. This study also provided some evidence for a reduction in cerebrovascular events. Despite the substantial reduction in the risk of coronary morbidity and mortality achieved by simvastatin, the risk is still substantial in the treated patients. For example, in the Scandinavian Simvastatin Survival Study, the 42% reduction in the risk of coronary death still left 5% of the treated patients to die of their disease over the course of this 5 year study. Further reduction of risk is clearly needed.

A more recent class of anti-hyperlipidemic agents that has emerged includes inhibitors of cholesterol absorption. Ezetimibe, the first compound to receive regulatory approval in this class, is currently marketed in the U.S. under the tradename ZETIA®. Ezetimibe has the following chemical structure and is described in U.S. Pat. No. Re. 37721 and U.S. Pat. No. 5,846,966:

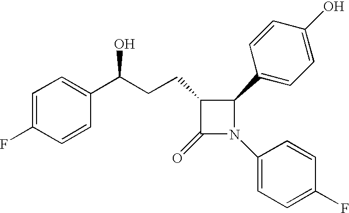

Sugar-substituted 2-azetidinones, including glucuronidated analogs of the following general structure:

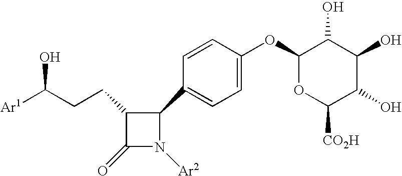

and methods for making them are disclosed in U.S. Pat. No. 5,756,470, wherein Ar¹ and Ar² are unsubstituted or substituted aryl groups.

Additional cholesterol absorption inhibitors are described in WO2002/066464 A1 (applied for by Kotobuki Pharmaceutical Co.), and US2002/0137689 A1 (Glombik et al.). WO2002/066464 A1 discloses hypolipidemic compounds of general formula

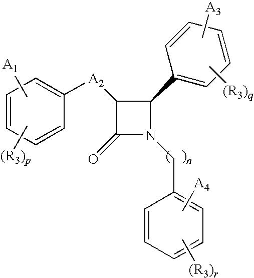

wherein, among other definitions, A₁, A₃ and A₄ can be

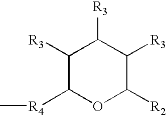

and wherein R₂ is —CH₂OH, —CH₂OC(O)—R₁, or —CO₂R₁; R₃ is —OH or —OC(O)R₁, and R₄ is —(CH₂)ₖR₅(CH₂)ᵢ— where k and i are zero or integers of one or more, and k+i is an integer of 10 or less; and R₅ is a single bond, —CH═CH—, —OCH₂—, carbonyl or —CH(OH).

US2002/0137689 A1 discloses hypolipidemic compounds of general formula

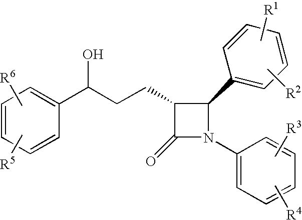

wherein, among other definitions, R¹, R², R³, R⁴, R⁵, R⁶ independently of one another can be (C₀-C₃₀)-alkylene-(LAG), where one or more carbon atoms of the alkylene radical may be replaced by —O—, —(C═O)—, —CH═CH—, —C≡C—, —N((C₁-C₆)-alkyl)-, —N((C₁-C₆)-alkylphenyl) or —NH—; and (LAG) is a sugar residue, disugar residue, trisugar residue, tetrasugar residue; a sugar acid, or an amino sugar.

In the ongoing effort to discover novel treatments for hyperlipidemia and atherosclerotic process, the instant invention provides novel cholesterol absorption inhibitors, described below.

### SUMMARY OF THE INVENTION

One object of the instant invention is to provide novel cholesterol absorption inhibitors of Formula I

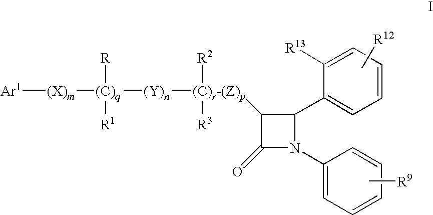

and the pharmaceutically acceptable salts thereof.

A second object of the instant invention is to provide a method for inhibiting cholesterol absorption comprising administering a therapeutically effective amount of a compound of Formula I to a patient in need of such treatment. Another object is to provide a method for reducing plasma cholesterol levels, especially LDL-cholesterol, and treating hypercholesterolemia comprising administering a therapeutically effective amount of a compound of Formula Ito a patient in need of such treatment.

As a further object, methods are provided for preventing or reducing the risk of developing atherosclerosis, as well as for halting or slowing the progression of atherosclerotic disease once it has become clinically evident, comprising the administration of a prophylactically or therapeutically effective amount, as appropriate, of a compound of Formula I to a patient who is at risk of developing atherosclerosis or who already has atherosclerotic disease. Another object of the present invention is the use of the compounds of the present invention for the manufacture of a medicament useful in treating, preventing or reducing the risk of developing these conditions. Other objects of this invention are to provide processes for making the compounds of Formula I and to provide novel pharmaceutical compositions comprising these compounds.

Additionally the compounds of this invention, particularly radioactive isotopes of the compounds of Formula I, can be used in screening assays, where the assay is designed to identify new cholesterol absorption inhibitors that have the same mechanism of action as ezetimibe. Additional objects will be evident from the following detailed description.

### DETAILED DESCRIPTION OF THE INVENTION

The novel cholesterol absorption inhibitors of the instant invention are compounds of structural Formula I

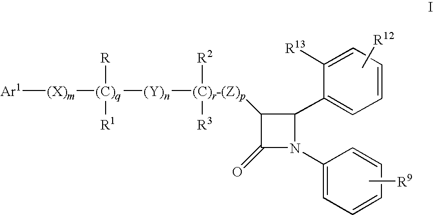

and the pharmaceutically acceptable salts thereof, wherein
Ar¹ is selected from the group consisting of aryl and R⁴-substituted aryl;
X, Y and Z are independently selected from the group consisting of —CH₂—, —CH(C₁₋₆alkyl)- and —C(C₁₋₆alkyl)₂—;
R is selected from the group consisting of —OR⁶, —O(CO)R⁶, —O(CO)OR⁸, —O(CO)NR⁶R⁷, a sugar residue, a disugar residue, a trisugar residue and a tetrasugar residue;
R¹ is selected from the group consisting of —H, —C₁₋₆alkyl and aryl, or R and R¹ together are oxo;
R² is selected from the group consisting of —OR⁶, —O(CO)R⁶, —O(CO)OR⁸ and —O(CO)NR⁶R⁷;
R³ is selected from the group consisting of —H, —C₁₋₆alkyl and aryl, or R² and R³ together are oxo;
q and r are integers each independently selected from 0 and 1 provided that at least one of q and r is 1;
m, n and p are integers each independently selected from 0, 1, 2, 3 and 4, provided that the sum of m, n, p, q and r is 1, 2, 3, 4, 5 or 6;
t is an integer selected from 0, 1 and 2;
R⁴ is 1-5 substituents independently selected at each occurrence from the group consisting of: —OR⁵, —O(CO)R⁵, —O(CO)OR⁸, —O—C₁₋₅alkyl-OR⁵, —O(CO)NR⁵R⁶, —NR⁵R⁶, —NR⁵(CO)R⁶, —NR⁵(CO)OR⁸, —NR⁵(CO)NR⁶R⁷, —NR⁵SO₂R⁸, —COOR⁵, —CONR⁵R⁶, —COR⁵, —SO₂NR⁵R⁶, —S(O)ₜR⁸, —O—C₁₋₁₀ alkyl-COOR⁵, —O—C₁₋₁₀alkyl-CONR⁵R⁶ and fluoro;
R⁵, R⁶ and R⁷ are independently selected at each occurrence from the group consisting of —H, —C₁₋₆alkyl, aryl and aryl-substituted —C₁₋₆alkyl;
R⁸ is selected from the group consisting of —C₁₋₆alkyl, aryl and aryl-substituted —C₁₋₆alkyl;
R⁹ is selected from the group consisting of —C₁₋₈alkyl-Hetcy, —(CH₂)₀₋₂CH═CH—C₀₋₆alkyl-Hetcy, —C≡C—C₀₋₆alkyl-Hetcy and —C₁₋₈alkyl-NH-Hetcy,
   Hetcy is selected from the group consisting of:
(a) a 5-membered aromatic or partially unsaturated heterocyclic ring containing 1 to 4 heteroatoms selected from 1 to 4 of N, zero to 1 of S, and zero to 1 of O, wherein the heterocyclic ring is optionally mono- or di-substituted with R¹⁴,
(b) a 6-membered aromatic heterocyclic ring containing 1 to 3 N heteroatoms, wherein the heterocyclic ring is optionally mono- or di-substituted with R¹⁴, and
(c) a 6-membered saturated heterocyclic ring containing 1 to 3 heteroatoms selected from 1-3 of N, zero to 1 of O, and zero to 1 of S(O)ₜ, and wherein the heterocyclic ring is optionally mono- or di-substituted with R¹⁴;
R^{10a} is —C₁₋₃alkyl optionally substituted with one or more substituents selected from the group consisting of —OH, phenyl and 1-3 of fluoro;
R¹⁰ is selected from the group consisting of —H and —C₁₋₃alkyl optionally substituted with one or more substituents selected from the group consisting of —OH, phenyl and 1-3 of fluoro;
R¹¹ is selected from the group consisting of —H and —C₁₋₃alkyl optionally substituted with one or more substituents selected from the group consisting of —OH, phenyl and 1-3 of fluoro;
R¹² is selected from the group consisting of —C₁₋₁₅alkyl mono- or poly-substituted with —OH, —C₂₋₁₅alkenyl mono- or poly-substituted with —OH, —C₂₋₁₅alkynyl mono- or poly-substituted with —OH, and —C₁₋₃alkyl-C₃₋₆cycloalkyl wherein each carbon in the cycloalkyl ring is optionally substituted with —OH;
R¹³ is selected from the group consisting of —H and —OH; and
R¹⁴ is independently selected at each occurrence from the group consisting of: R^{10a}, —C₁₋₃alkyl-COOR¹⁰, —C₁₋₃alkyl-C(O)NR¹⁰R¹¹, —C₁₋₃alkyl-SO₂—R^{10a}, —C₁₋₃alkyl-O—R^{10a}, —COOR¹⁰, —OC(O)—R^{10a}, —C(O)NR¹⁰R¹¹, —NR¹⁰R¹¹, —CN, —OH and oxo.

In an embodiment of this invention, referred to herein as Embodiment A, are compounds of Formula I wherein R⁹ is selected from the group consisting of —C₁₋₈alkyl-Hetcy, —(CH₂)₀₋₂CH═CH—C₁₋₆alkyl-Hetcy, —C≡C—C₁₋₆alkyl-Hetcy and —C₁₋₈alkyl-NH-Hetcy and R¹⁴ is independently selected at each occurrence from the group consisting of R^{10a}, —C₁₋₃alkyl-COOR¹⁰, —C₁₋₃alkyl-C(O)NR¹⁰R¹¹, —C₁₋₃alkyl-SO₂—R^{10a}, —C₁₋₃alkyl-O—R^{10a}, —COOR¹⁰, —OC(O)—R^{10a}, —C(O)NR¹⁰R¹¹, —NR¹⁰R¹¹, —OH and oxo.

In another embodiment of this invention are compounds of Formula I and Embodiment A wherein the sum of m, q and n is 1, 2, 3, 4, or 5 when p is 0 and r is 1.

In another embodiment of this invention are compounds of Formula I and Embodiment A wherein r is zero and m is zero; and more particularly wherein r is zero, m is zero, q is 1, n is 1 and p is 1.

In a another embodiment of this invention are compounds Formula I and Embodiment A having structural Formula Ia,

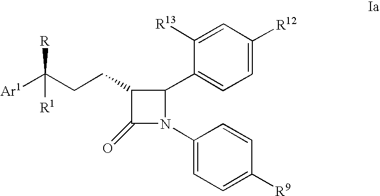

and the pharmaceutically acceptable salts thereof, wherein the variables (Ar¹, R, R¹, R⁹, R¹², R¹³) are as defined in Formula I or Embodiment A.

In another embodiment of this invention are compounds Formula I and Embodiment A having structural Formula Ib,

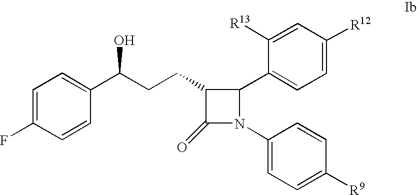

and the pharmaceutically acceptable salts thereof, wherein the variables (R⁹, R¹², R¹³) are as defined in Formula I or Embodiment A.

In another embodiment of this invention are compounds of Formula I, Ia or Embodiment A wherein Ar¹ is selected from the group consisting of aryl and R⁴-substituted aryl wherein R⁴ is 1-2 substituents independently selected at each occurrence from the group consisting of: —OR⁵, —O(CO)R⁵, —O(CO)OR⁸, —O—C₁₋₅alkyl-OR⁵, —O(CO)NR⁵R⁶, —NR⁵R⁶, —NR⁵(CO)R⁶, —NR⁵(CO)OR⁸, —NR⁵(CO)NR⁶R⁷, —NR⁵SO₂R⁸, —COOR⁵, —CONR⁵R⁶, —COR⁵, —SO₂NR⁵R⁶, —S(O)ₜR⁸, —O—C₁₋₁₀alkyl-COOR⁵, —O—C₁₋₁₀alkyl-CONR⁵R⁶ and fluoro. In a class of this embodiment, Ar¹ is unsubstituted, mono- or di-substituted phenyl. In a sub-class, Ar¹ is phenyl mono-substituted with fluoro, and particularly 4-fluoro-phenyl.

In another embodiment of this invention are compounds of Formula I, Ia or Embodiment A wherein R is —OR⁶; in a class of this embodiment, R is —OH.

In another embodiment of this invention are compounds of Formula I, Ia or Embodiment A wherein R¹ is —H.

In another embodiment of this invention are compounds of Formula I or Embodiment A wherein R² is —OR⁶; in a class of this embodiment, R² is —OH.

In another embodiment of this invention are compounds of Formula I or Embodiment A wherein R³ is —H.

In another embodiment of this invention are compounds of Formula I, Ia, Ib or Embodiment A wherein R⁹ is —C₁₋₈alkyl-Hetcy. In a class of this embodiment R⁹ is —C₂₋₃alkyl-Hetcy. More particularly, the alkyl portion of R⁹ which links Hetcy to the phenyl ring is n-alkyl.

In another embodiment of this invention are compounds of Formula I, Ia, Ib or Embodiment A wherein R⁹ is —(CH₂)₀₋₂CH═CH—C₀₋₆alkyl-Hetcy. In a class of this embodiment R⁹ is —CH═CH—C₀₋₆ n-alkyl-Hetcy, and more particularly it is —CH═CH—C₀₋₁-alkyl-Hetcy.

In another embodiment of this invention are compounds of Formula I, Ia, Ib or Embodiment A wherein R⁹ is —C≡C—C₀₋₆alkyl-Hetcy. In a class of this embodiment R⁹ is —C≡C—C₀₋₆ n-alkyl-Hetcy, and more particularly it is —C≡C—C₀₋₁alkyl-Hetcy.

In another embodiment of this invention are compounds of Formula I, Ia, Ib or Embodiment A wherein R⁹ is —C₁₋₈alkyl-NH-Hetcy. In a class of this embodiment R⁹ is —C₁₋₃alkyl-NH-Hetcy.

In another embodiment of this invention are compounds of Formula I, Ia, Ib or Embodiment A wherein Hetcy is a 5-membered aromatic or partially unsaturated heterocyclic ring containing 1 to 4 heteroatoms selected from 1 to 4 of N, zero to 1 of S, and zero to 1 of O, wherein the heterocyclic ring is optionally mono- or di-substituted with R¹⁴. Examples of such heterocyclic rings within the meaning of Hetcy include but are not limited to the following, each of which may be optionally mono- or di-substituted with R¹⁴:

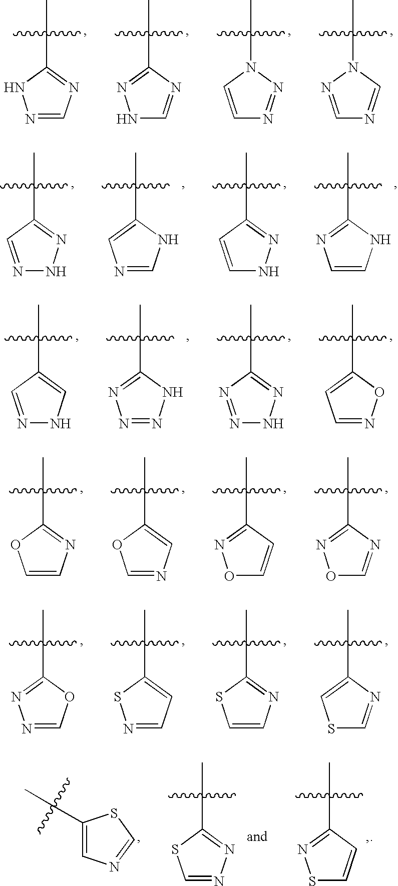

In another embodiment of this invention are compounds of Formula I, Ia, Ib or Embodiment A wherein Hetcy is a 6-membered aromatic heterocyclic ring containing 1 to 3 N heteroatoms, and particularly wherein the ring contains 1-2 of N, wherein the heterocyclic ring is optionally mono- or di-substituted with R¹⁴. Examples of such heterocyclic rings within the meaning of Hetcy include but are not limited to the following, each of which may be optionally mono- or di-substituted with R¹⁴:

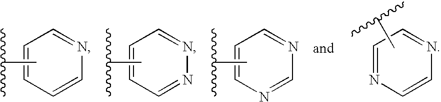

In another embodiment of this invention are compounds of Formula I, Ia, Ib or Embodiment A wherein Hetcy is a 6-membered saturated heterocyclic ring containing 1 to 3 heteroatoms selected from 1-3 of N, zero to 1 of 0, and zero to 1 of S(O)ₜ, wherein the heterocyclic ring is optionally substituted with R¹⁴. Examples of such heterocyclic rings within the meaning of Hetcy include but are not limited to the following, each of which may be optionally mono- or di-substituted with R¹⁴:

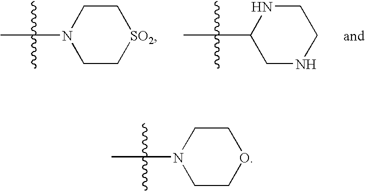

In another embodiment of this invention are compounds of Formula I, Ia, Ib or Embodiment A wherein R¹⁰ is selected from —H and methyl.

In another embodiment of this invention are compounds of Formula I, Ia, Ib or Embodiment A wherein R¹¹ is selected from —H and methyl.

In another embodiment of this invention are compounds of Formula I, Ia, Ib or Embodiment A wherein R¹² is —C₁₋₁₅alkyl mono- or poly-substituted with —OH. In a class of this embodiment, R¹² is —C₁₋₈alkyl mono- or poly-substituted with —OH. In a sub-class of this class, R¹² is —C₃₋₆ alkyl mono- or poly-substituted with —OH. In a further sub-class of this class, R¹² is —(CH₂)₂₋₃—C(OH)(CH₂OH)₂.

In another embodiment of this invention are compounds of Formula I, Ia, Ib or Embodiment A wherein R¹² is —C₂₋₁₅alkenyl mono- or poly-substituted with —OH. In a class of this embodiment, R¹² is —C₂₋₈alkenyl mono- or poly-substituted with —OH. In a sub-class of this class, R¹² is —C₃₋₆ alkenyl mono- or poly-substituted with —OH. In a further sub-class of this class, R¹² is —(CH₂)₀₋₁—CH═CH—C(OH)(CH₂OH)₂.

In another embodiment of this invention are compounds of Formula I, Ia, Ib or Embodiment A wherein R¹² is —C₂₋₁₅alkynyl mono- or poly-substituted with —OH. In a class of this embodiment, R¹² is —C₂₋₈alkynyl mono- or poly-substituted with —OH. In a sub-class of this class, R¹² is —C₃₋₆ alkynyl mono- or poly-substituted with —OH. In a further sub-class of this class, R¹² is —(CH₂)₀₋₁—C≡C—C(OH)(CH₂OH)₂.

When any variable (e.g., X, Y, Z, R⁵, R⁶, R¹⁰, R¹¹, R¹⁴, etc.) can be present more than once in a generic structure, its definition is independently selected at each occurrence, so it may be defined the same or differently at each point of attachment.

Each embodiment, class or sub-class described above for each variable (i.e., Ar¹, R, R¹, R⁹, R¹², etc.) in Formulas I, Ia and Ib may be combined with one or more of the embodiments, classes or sub-classes described above for one or more other variables, and all such generic sub-combinations are included within the scope of this invention.

As used herein “alkyl” is intended to include both branched- and straight-chain saturated aliphatic hydrocarbon groups having the specified number of carbon atoms. Examples of alkyl groups include, but are not limited to, methyl (Me), ethyl (Et), n-propyl (Pr), n-butyl (Bu), n-pentyl, n-hexyl, and the isomers thereof such as isopropyl (i-Pr), isobutyl (i-Bu), secbutyl (s-Bu), tertbutyl (t-Bu), 1-methylpropyl, 2-methylbutyl, 3-methylbutyl, isopentyl, isohexyl and the like.

“Alkenyl” means carbon chains which contain at least one carbon-carbon double bond, and which may be linear or branched or combinations thereof. Examples of alkenyl include vinyl, allyl, isopropenyl, pentenyl, hexenyl, heptenyl, 1-propenyl, 2-butenyl, 2-methyl-2-butenyl, and the like.

“Alkynyl” means carbon chains which contain at least one carbon-carbon triple bond, and which may be linear or branched or combinations thereof. Examples of alkynyl include ethynyl, propargyl, 3-methyl-1-pentynyl, 2-heptynyl and the like.

“Cycloalkyl” means a monocyclic saturated carbocyclic ring. Examples of cycloalkyl include, but are not limited to, cyclopropyl, cyclobutyl, cyclopentyl, cyclohexyl, and the like.

Certain alkyl, alkenyl and alkynyl groups (collectively referred to as “alk” chains), are defined herein as being “mono- or poly-substituted with —OH,” meaning that one or more hydroxyl substituents is present on the alk chain, and that each carbon atom available for substitution in the alk chain may independently be unsubstituted or mono-substituted with hydroxyl provided that at least one carbon atom is substituted with hydroxyl. This encompasses —CH₂OH and longer alk chains where every available carbon atom is mono-substituted with hydroxyl as well as those where fewer than all available carbon atoms are mono-substituted with hydroxyl. In said alkenyl chains, it is preferred that the unsaturated carbons are not substituted with hydroxyl, although such carbons can be converted to saturated hydroxyl-substituted carbons. The alk chains that are mono- or poly-substituted with —OH can contain up to 15 carbons as defined in R¹², including straight and branched chains containing fewer carbons, for example but not limited to 1-8 carbons (for alkyl), 2-8 carbons, 3-8 carbons, 4-8 carbons, 5-8 carbons, 5-6 carbons, etc.

Hydroxyl protecting groups may be used on intermediates during the synthetic procedures for making final products within the scope of this invention. Suitable protecting groups for the hydroxyl groups, for example those in R¹² and R¹³, include but are not limited to those that are known to be useful as hydroxyl protecting groups, such as for example benzyl, acetyl, benzoyl, tert-butyldiphenylsilyl, trimethylsilyl, para-methoxybenzyl, benzylidine, dimethylacetal and methoxy methyl. Conditions required to selectively add and remove such protecting groups are found in standard textbooks such as Greene, T, and Wuts, P. G. M., Protective Groups in Organic Synthesis, John Wiley & Sons, Inc., New York, N.Y., 1999.

As used herein, “aryl” is intended to include phenyl (Ph), naphthyl, indenyl, tetrahydronaphthyl or indanyl. Phenyl is preferred.

The terms “heterocycle” and derivatives thereof such as “heterocyclyl” and “heterocyclic ring” mean an aromatic, partially unsaturated or saturated ring containing one or more carbon atoms and one or more heteroatoms such as nitrogen, oxygen and sulfur, but may be more specifically defined where appropriate in the specification, for example with respect to degree of saturation, number of members (i.e. atoms) in the ring and/or the type and quantity of heteroatoms in the ring. The point of attachment in a compound structure may be via any carbon or nitrogen in the heterocyclic ring which results in the creation of a stable structure, unless specified otherwise. The heterocyclic ring may be substituted on any available carbon or nitrogen in the ring which results in the creation of a stable structure, unless specified otherwise.

Compounds of Formula I may contain one or more asymmetric (i.e., chiral) centers and can thus occur as racemates and racemic mixtures, single enantiomers, enantiomeric mixtures, diastereomeric mixtures and individual diastereomers. All such isomeric forms of the compounds of Formula I are included within the scope of this invention. Furthermore, some of the crystalline forms for compounds of the present invention may exist as polymorphs and as such all amorphous and crystalline forms are intended to be included in the scope of the present invention. In addition, some of the compounds of the instant invention may form solvates with water or organic solvents. Such hydrates and solvates are also encompassed within the scope of this invention.

Some of the compounds described herein may contain olefinic double bonds, and unless specified otherwise, are meant to include both E and Z geometric isomers, singly or as a mixture.

Some of the compounds encompassed herein may exist as tautomers, e.g., keto-enol tautomers. For the purpose of illustration, when Hetcy is a 5-membered heterocyclic substituted with oxo, the resulting compound may be capable of tautomerism, as exemplified below:

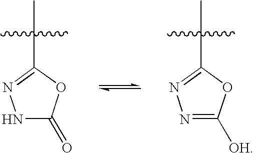

Where compounds of this invention are capable of tautomerization, all individual tautomers as well as mixtures thereof are included in the scope of this invention.

Reference to the compounds of this invention as those of “Formula I” herein also includes compounds defined by the scope of each of the sub-generic descriptions such as Formulas Ia, and Ib, as well as individual compounds within the scope of any of these sub-generic descriptions, unless in context a structural sub-group of compounds is being addressed as in, for example, the synthetic description of how to make certain compounds within a structural sub-group. Reference to the compounds of this invention as those of “Formula I,” “Formula Ia,” and “Formula Ib” or any other generic structural formula used herein is intended to encompass compounds falling within the scope of each of these structural formulas including pharmaceutically acceptable salts and esters thereof where such salts and esters are possible. Herein, the term “pharmaceutically acceptable salts” means non-toxic salts of the compounds employed in this invention which are generally prepared by reacting the free acid with a suitable organic or inorganic base, particularly those formed from cations such as sodium, potassium, aluminum, calcium, lithium, magnesium, zinc and tetramethylammonium, as well as those salts formed from amines such as ammonia, ethylenediamine, N-methylglucamine, lysine, arginine, ornithine, choline, N,N′-dibenzylethylenediamine, chloroprocaine, diethanolamine, procaine, N-benzylphenethylamine, 1-p-chlorobenzyl-2-pyrrolidine-1′-yl-methylbenzimidazole, diethylamine, piperazine, morpholine, 2,4,4-trimethyl-2-pentamine and tris(hydroxymethyl)aminomethane.

When the compound of the present invention is basic, salts may be prepared from pharmaceutically acceptable non-toxic acids, including inorganic and organic acids. Such acids include acetic, trifluoroacetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like.

Also, in the case of a carboxylic acid (—COOH) or alcohol group being present in the compounds of this invention, pharmaceutically acceptable esters of carboxylic acid derivatives, such as —C1-4 alkyl, —C1-4 alkyl substituted with phenyl, acetylamino and pivaloyloxymethyl, or acyl derivatives of alcohols such as O-acetyl, O-pivaloyl, O-benzoyl, O-dimethylamino and O-acetylamino, can be employed. Included within the scope of this invention are those esters and acyl groups known in the art for modifying the solubility or hydrolysis characteristics of a compound for use as sustained-release or prodrug formulations.

The term “patient” includes mammals, especially humans, who use the instant active agents for the prevention or treatment of a medical condition. Administering of the drug to the patient includes both self-administration and administration to the patient by another person. The patient may be in need of treatment for an existing disease or medical condition, or may desire prophylactic treatment to prevent or reduce the risk for diseases and medical conditions affected by inhibition of cholesterol absorption.

The term “therapeutically effective amount” is intended to mean that amount of a pharmaceutical drug that will elicit the biological or medical response of a tissue, a system, animal or human that is being sought by a researcher, veterinarian, medical doctor or other clinician. The term “prophylactically effective amount” is intended to mean that amount of a pharmaceutical drug that will prevent or reduce the risk of occurrence of the biological or medical event that is sought to be prevented in a tissue, a system, animal or human by a researcher, veterinarian, medical doctor or other clinician. Particularly, the dosage a patient receives can be selected so as to achieve the amount of LDL cholesterol lowering desired; the dosage a patient receives may also be titrated over time in order to reach a target LDL level. The dosage regimen utilizing a compound of the instant invention is selected in accordance with a variety of factors including type, species, age, weight, sex and medical condition of the patient; the severity of the condition to be treated; the potency of the compound chosen to be administered; the route of administration; and the renal and hepatic function of the patient. A consideration of these factors is well within the purview of the ordinarily skilled clinician for the purpose of determining the therapeutically effective or prophylactically effective dosage amount needed to prevent, counter, or arrest the progress of the condition.

The compounds of the instant invention are cholesterol absorption inhibitors and are useful for reducing plasma cholesterol levels, particularly reducing plasma LDL cholesterol levels, when used either alone or in combination with another active agent, such as an anti-atherosclerotic agent, and more particularly a cholesterol biosynthesis inhibitor, for example an HMG-CoA reductase inhibitor. Thus the instant invention provides methods for inhibiting cholesterol absorption and for treating lipid disorders including hypercholesterolemia, comprising administering a therapeutically effective amount of a compound of Formula Ito a person in need of such treatment. The term hypercholesterolemia includes but is not limited to homozygous familial hypercholesterolemia (HoFH) and heterozygous familial hypercholesterolemia (HeFH) and therefore the compounds of Formula I can be used treat HoHF and HeHF patients. These compounds can also be used for the treatment of mixed hyperlipidemia which is characterized by an elevated LDL cholesterol level and elevated triglycerides level along with an undesirably low HDL cholesterol level. Compounds of Formula I can also be used to treat or prevent sitosterolemia and/or to lower the concentration of one or more sterols other than cholesterol in the plasma or tissue of a patient.

Further provided are methods for preventing or reducing the risk of developing atherosclerosis, as well as for halting or slowing the progression of atherosclerotic disease once it has become clinically evident, comprising the administration of a prophylactically or therapeutically effective amount, as appropriate, of a compound of Formula Ito a mammal who is at risk of developing atherosclerosis or who already has atherosclerotic disease.

Atherosclerosis encompasses vascular diseases and conditions that are recognized and understood by physicians practicing in the relevant fields of medicine. Atherosclerotic cardiovascular disease including restenosis following revascularization procedures, coronary heart disease (also known as coronary artery disease or ischemic heart disease), cerebrovascular disease including multi-infarct dementia, and peripheral vessel disease including erectile dysfunction are all clinical manifestations of atherosclerosis and are therefore encompassed by the terms “atherosclerosis” and “atherosclerotic disease.”

A compound of Formula I may be administered to prevent or reduce the risk of occurrence, or recurrence where the potential exists, of a coronary heart disease event, a cerebrovascular event, and/or intermittent claudication. Coronary heart disease events are intended to include CHD death, myocardial infarction (i.e., a heart attack), and coronary revascularization procedures. Cerebrovascular events are intended to include ischemic or hemorrhagic stroke (also known as cerebrovascular accidents) and transient ischemic attacks. Intermittent claudication is a clinical manifestation of peripheral vessel disease. The term “atherosclerotic disease event” as used herein is intended to encompass coronary heart disease events, cerebrovascular events, and intermittent claudication. It is intended that persons who have previously experienced one or more non-fatal atherosclerotic disease events are those for whom the potential for recurrence of such an event exists.

Accordingly, the instant invention also provides a method for preventing or reducing the risk of a first or subsequent occurrence of an atherosclerotic disease event comprising the administration of a prophylactically effective amount of a compound of Formula I to a patient at risk for such an event. The patient may or may not have atherosclerotic disease at the time of administration, or may be at risk for developing it.

Persons to be treated with the instant therapy include those at risk of developing atherosclerotic disease and of having an atherosclerotic disease event. Standard atherosclerotic disease risk factors are known to the average physician practicing in the relevant fields of medicine. Such known risk factors include but are not limited to hypertension, smoking, diabetes, low levels of high density lipoprotein (HDL) cholesterol, and a family history of atherosclerotic cardiovascular disease. Published guidelines for determining those who are at risk of developing atherosclerotic disease can be found in: Executive Summary of the Third Report of the National Cholesterol Education Program (NCEP) Expert Panel on Detection, Evaluation, and Treatment of High Blood Cholesterol in Adults (Adult Treatment Panel III), JAMA, 2001; 285 pp. 2486-2497. People who are identified as having one or more of the above-noted risk factors are intended to be included in the group of people considered at risk for developing atherosclerotic disease. People identified as having one or more of the above-noted risk factors, as well as people who already have atherosclerosis, are intended to be included within the group of people considered to be at risk for having an atherosclerotic disease event.

The oral dosage amount of the compound of Formula I is from about 0.1 to about 30 mg/kg of body weight per day, preferably about 0.1 to about 15 mg/kg of body weight per day. For an average body weight of 70 kg, the dosage level is therefore from about 5 mg to about 1000 mg of drug per day. However, dosage amounts will vary depending on factors as noted above, including the potency of the particular compound. Although the active drug of the present invention may be administered in divided doses, for example from two to four times daily, a single daily dose of the active drug is preferred. As examples, the daily dosage amount may be selected from, but not limited to, 5 mg, 10 mg, 15 mg, 20 mg, 25 mg, 30 mg, 35 mg, 40 mg, 45 mg, 50 mg, 75 mg, 80 mg, 100 mg and 200 mg.

The active drug employed in the instant therapy can be administered in such oral forms as tablets, capsules, pills, powders, granules, elixirs, tinctures, suspensions, syrups, and emulsions. Oral formulations are preferred, and particularly solid oral formulations such as tablets.

For compounds of Formula I, administration of the active drug can be via any pharmaceutically acceptable route and in any pharmaceutically acceptable dosage form. This includes the use of oral conventional rapid-release, time controlled-release and delayed-release (such enteric coated) pharmaceutical dosage forms. Additional suitable pharmaceutical compositions for use with the present invention are known to those of ordinary skill in the pharmaceutical arts; for example, see Remington's Pharmaceutical Sciences, Mack Publishing Co., Easton, Pa.

In the methods of the present invention, the active drug is typically administered in admixture with suitable pharmaceutical diluents, excipients or carriers (collectively referred to herein as “carrier” materials) suitably selected with respect to the intended form of administration, that is, oral tablets, capsules, elixirs, syrups and the like, and consistent with conventional pharmaceutical practices.

For instance, for oral administration in the form of a tablet or capsule, the active drug component can be combined with a non-toxic, pharmaceutically acceptable, inert carrier such as lactose, starch, sucrose, glucose, modified sugars, modified starches, methyl cellulose and its derivatives, dicalcium phosphate, calcium sulfate, mannitol, sorbitol and other reducing and non-reducing sugars, magnesium stearate, steric acid, sodium stearyl fumarate, glyceryl behenate, calcium stearate and the like. For oral administration in liquid form, the drug components can be combined with non-toxic, pharmaceutically acceptable inert carrier such as ethanol, glycerol, water and the like. Moreover, when desired or necessary, suitable binders, lubricants, disintegrating agents and coloring and flavoring agents can also be incorporated into the mixture. Stabilizing agents such as antioxidants, for example butylated hydroxyanisole (BHA), 2,6-di-tert-butyl-4-methylphenol (BHT), propyl gallate, sodium ascorbate, citric acid, calcium metabisulphite, hydroquinone, and 7-hydroxycoumarin, particularly BHA, propyl gallate and combinations thereof, can also be added to stabilize the dosage forms. When a compound of Formula I is formulated together with an HMG-CoA reductase inhibitor such as simvastatin, the use of at least one stabilizing agent is preferred in the composition. Other suitable components include gelatin, sweeteners, natural and synthetic gums such as acacia, tragacanth or alginates, carboxymethylcellulose, polyethylene glycol, waxes and the like.

The instant invention also encompasses a process for preparing a pharmaceutical composition comprising combining a compound of Formula I with a pharmaceutically acceptable carrier. Also encompassed is the pharmaceutical composition which is made by combining a compound of Formula I with a pharmaceutically acceptable carrier.

One or more additional active agents may be administered in combination with a compound of Formula I, and therefore an embodiment of the instant invention encompasses a drug combination. The drug combination encompasses a single dosage formulation comprised of the compound of Formula I and additional active agent or agents, as well as administration of each of the compound of Formula I and the additional active agent or agents in separate dosage formulations, which allows for concurrent or sequential administration of the active agents. The additional active agent or agents can be lipid modifying agents, particularly a cholesterol biosynthesis inhibitor such as an HMG-CoA reductase inhibitor, or agents having other pharmaceutical activities, or agents that have both lipid-modifying effects and other pharmaceutical activities. Examples of HMG-CoA reductase inhibitors useful for this purpose include statins in their lactonized or dihydroxy open acid forms and pharmaceutically acceptable salts and esters thereof, including but not limited to lovastatin (MEVACOR®; see U.S. Pat. No. 4,342,767); simvastatin (ZOCOR®; see U.S. Pat. No. 4,444,784); dihydroxy open-acid simvastatin, particularly the ammonium or calcium salts thereof; pravastatin, particularly the sodium salt thereof (PRAVACOL®; see U.S. Pat. No. 4,346,227); fluvastatin particularly the sodium salt thereof (LESCOL®; see U.S. Pat. No. 5,354,772); atorvastatin, particularly the calcium salt thereof (LIPITOR®; see U.S. Pat. No. 5,273,995); rosuvastatin (CRESTOR®; see U.S. Pat. No. 5,260,440); and pitavastatin also referred to as NK-104 (see PCT international publication number WO 97/23200). Examples of additional active agents which may be employed include but are not limited to one or more of FLAP inhibitors; 5-lipoxygenase inhibitors; additional cholesterol absorption inhibitors such as ezetimibe (ZETIA®), described in U.S. Pat. No. Re. 37721 and U.S. Pat. No. 5,846,966; cholesterol ester transfer protein (CETP) inhibitors, for example JTT-705 and torcetrapib, also known as CP529,414; HMG-CoA synthase inhibitors; squalene epoxidase inhibitors; squalene synthetase inhibitors (also known as squalene synthase inhibitors); acyl-coenzyme A: cholesterol acyltransferase (ACAT) inhibitors including selective inhibitors of ACAT-1 or ACAT-2 as well as dual inhibitors of ACAT1 and -2; microsomal triglyceride transfer protein (MTP) inhibitors; niacin; niacin receptor agonists such as acipimox and acifran, as well as niacin receptor partial agonists; LDL (low density lipoprotein) receptor inducers; platelet aggregation inhibitors, for example glycoprotein IIb/IIIa fibrinogen receptor antagonists and aspirin; human peroxisome proliferator activated receptor gamma (PPARγ) agonists including the compounds commonly referred to as glitazones for example pioglitazone and rosiglitazone and, including those compounds included within the structural class known as thiazolidinediones as well as those PPARγ agonists outside the thiazolidinedione structural class; PPARα agonists such as clofibrate, fenofibrate including micronized fenofibrate, and gemfibrozil; PPAR dual α/γ agonists; vitamin B₆ (also known as pyridoxine) and the pharmaceutically acceptable salts thereof such as the HCl salt; vitamin B₁₂ (also known as cyanocobalamin); folic acid or a pharmaceutically acceptable salt or ester thereof such as the sodium salt and the methylglucamine salt; anti-oxidant vitamins such as vitamin C and E and beta carotene; beta-blockers; angiotensin II antagonists such as losartan; angiotensin converting enzyme inhibitors such as enalapril and captopril; calcium channel blockers such as nifedipine and diltiazam; endothelian antagonists; agents that enhance ABC1 gene expression; FXR ligands including both inhibitors and agonists; and LXR ligands including both inhibitors and agonists of all sub-types of this receptor, e.g. LXRα and LXRβ; bisphosphonate compounds such as alendronate sodium; and cyclooxygenase-2 inhibitors such as rofecoxib, celecoxib and valdecoxib.

A therapeutically or prophylactically effective amount, as appropriate, of a compound of Formula I can be used for the preparation of a medicament useful for treatments described above, e.g., inhibiting cholesterol absorption, as well as for treating and/or reducing the risk for diseases and conditions affected by inhibition of cholesterol absorption, such as treating lipid disorders, preventing or reducing the risk of developing atherosclerotic disease, halting or slowing the progression of atherosclerotic disease once it has become clinically manifest, and preventing or reducing the risk of a first or subsequent occurrence of an atherosclerotic disease event. For example, the medicament may be comprised of about 5 mg to about 1000 mg of a compound of Formula I. The medicament comprised of a compound of Formula I may also be prepared with one or more additional active agents, such as those described supra.

Compounds of this invention were determined to inhibit cholesterol absorption employing the Cholesterol Absorption Assay in Rat, below. This assay involves comparing a test compound to ezetimibe with respect to their ability to inhibit cholesterol absorption in rat. Both ezetimibe and the tested compounds of this invention inhibited cholesterol absorption by >90% at the highest dose tested. Compounds of this inventions that were tested had an ID 50<1 mg/kg.

Cholesterol Absorption Assay in Rats: CD male rats (n=5/group), aged 5 weeks, were dosed orally with 0.5 ml 0.25% methyl cellulose solution with or without test compound or ezetimibe (0.0003 to 1 mg/kg). 0.5 to 16 hrs later all of the rats were dosed orally with 0.5 ml INTRALIPID® containing 5 μCi [³H]-cholesterol per rat. Five hours later, the animals were euthanized, and liver and blood were collected. Cholesterol counts in liver and plasma were determined, and percent inhibition of cholesterol absorption was calculated.

The compounds of structural Formula I of the present invention can be prepared according to the procedures of the following Scheme and Examples, using appropriate materials, and are further exemplified by specific examples which follow. Moreover, by utilizing the procedures described herein, one of ordinary skill in the art can readily prepare additional compounds of the present invention claimed herein. The compounds illustrated in the examples are not, however, to be construed as forming the only genus that is considered as the invention. The Examples further illustrate details for the preparation of the compounds of the present invention. Those skilled in the art will readily understand that known variations of the conditions and processes of the following preparative procedures can be used to prepare these compounds.

A variety of chromatographic techniques may be employed in the preparation of the compounds. These techniques include, but are not limited to: High Performance Liquid Chromatography (HPLC) including normal-reversed- and chiral-phase; Medium Pressure Liquid Chromatography (MPLC), Super Critical Fluid Chromatography; preparative Thin Layer Chromatography (prep TLC); flash chromatography with silica gel or reversed-phase silica gel; ion-exchange chromatography; and radial chromatography. All temperatures are degrees Celsius unless otherwise noted.

Some abbreviations used herein include:
Ac=Acyl (CH₃C(O)—); Aq=Aqueous; Bn=Benzyl; Br=Bromide; C.=Celsius; calc.=Calculated; DCM=dichloromethane; DIEA=N,N-diisopropylethylamine; DMAP=4-dimethylaminopyridine; DMF=N,N-dimethylformamide; equiv.=Equivalent(s); ES-MS=Electron Spray Ion-Mass Spectroscopy; EtOAc=Ethyl acetate; H=Hours(s); HPLC=High pressure liquid chromatography; I=iodide; Min=Minute(s); Mp or Mpt=Melting point; MPLC=Medium pressure liquid chromatography; MS=Mass spectrum; NMO=N-methylmorpholine N-oxide; OTf=triflate; Prep.=Preparative; r.t. (or rt or RT)=Room temperature; sat.=Saturated; TBAI=Tetrabutylammonium iodide; TBS=Tert-butyl dimethylsilyl; TEA=Triethyl amine; TFA=Trifluoroacetic acid; THF=Tetrahydrofuran; TLC=Thin layer chromatography; TMS=Trimethylsilyl.

The general Schemes below illustrate a method for the syntheses of compounds of the present invention. All substituents and variables (e.g., R¹, R², Ar¹, X, Y, etc.) are as defined above in Formula I unless indicated otherwise. In the schemes, R^{12a} represents an alkyl group which is mono- or poly-substituted with hydroxyl or protected hydroxyl.

In Scheme I, the intermediate I-1 can be converted to I-2 by treatment with guanidine and triethylamine in methanol to selectively remove the phenolic acetate; then converting the intermediate phenol to the triflate via treatment with bis(trifluoromethylsulfonyl)amino pyridine in the presence of either triethylamine or N,N diisopropyl-N-ethyl amine in dichloromethane medium. Intermediate I-2 is then treated with a terminal alkyne of type I-3 containing the R^{12a} group in the presence of a suitable palladium catalyst such as tetrakistriphenylphosphine palladium(0) or [1,1′-bis(diphenylphosphino)ferrocene]dichloropalladium(II) or the like, and copper(I) iodide and an initiator such as tetra-n-butylammonium iodide. The reaction is usually performed in an inert organic solvent such as DMF, between room temperature and 100° C., for a period of 6-48 h, and the product is an internal alkyne of structural formula I-4. R^{12a} group within intermediate I-3 may possess either hydroxyl-protected or unprotected alkynyl-R^{12a} derivative I-3. Examples of hydroxyl protecting groups (PG) include, for example, benzyl, acetate, acetal or any other suitable oxygen protecting group, or combinations thereof, compatible with earlier or subsequent chemical reactions. As an example, R^{12a} includes but is not limited to —C₁₋₆alkyl-OBn and

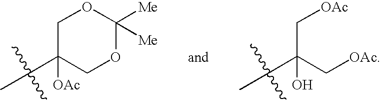

The resulting triflate I-4 is treated with an alkynyl-(CH₂)ₙ-heteroaryl group of type I-5 in the presence of a suitable palladium catalyst such as tetrakistriphenylphosphine palladium(0) and copper(I) iodide with an initiator such as tetrabutylammonium iodide. The reaction is usually performed in an inert organic solvent such as DMF, at RT to 50° C., for a period of 1 to 5 hrs, and the product possesses an alkynyl-(CH₂)ₙ-heteroaryl group of structure I-6. Hydrogenation of this bis-alkyne intermediate I-6 by treatment with 10% palladium on carbon catalyst under hydrogen atmosphere in a solvent such as ethyl acetate over 15-24 hours may achieve hydrogenation of the triple bonds along with the removal of any benzyl protecting groups in I-6, except for substituent R¹³ in which the benzyl protection survives these hydrogenation conditions. An additional deprotection step may be included if there are useful protecting groups on the heteroaryl group know to those skilled in the art necessary to allow the chemistry to proceed in a facile fashion. These protecting groups may include trityl groups, t-butylcarbamate groups or other groups suitable for the protection of heterocyclic compounds or the functional groups attached to the heterocyclic group known to those skilled in the art. Hydrolysis or cleavage of any remaining hydroxyl protecting groups may be performed at this time, or non-benzylic protecting groups can be removed prior to the hydrogenation step. For example, diols protected as acetals that are contained in R^{12a} may be removed by treatment with aqueous acid. When R^{12a} contains one or more acetate groups, deprotection with potassium cyanide or potassium trimethylsilanoate in an alcohol solvent such as ethanol at ambient temperature or heated to 50° C. for 1-2 hours affords the free hydroxyl groups to form compounds of the present invention I-7. When R¹³ is the 2-benzyloxy substituent, a second deprotection step using 10% palladium on carbon in ethanol under hydrogen atmosphere is required as a final deprotection to afford the 2-hydroxy substituted phenyl as in the structure of type I-7.

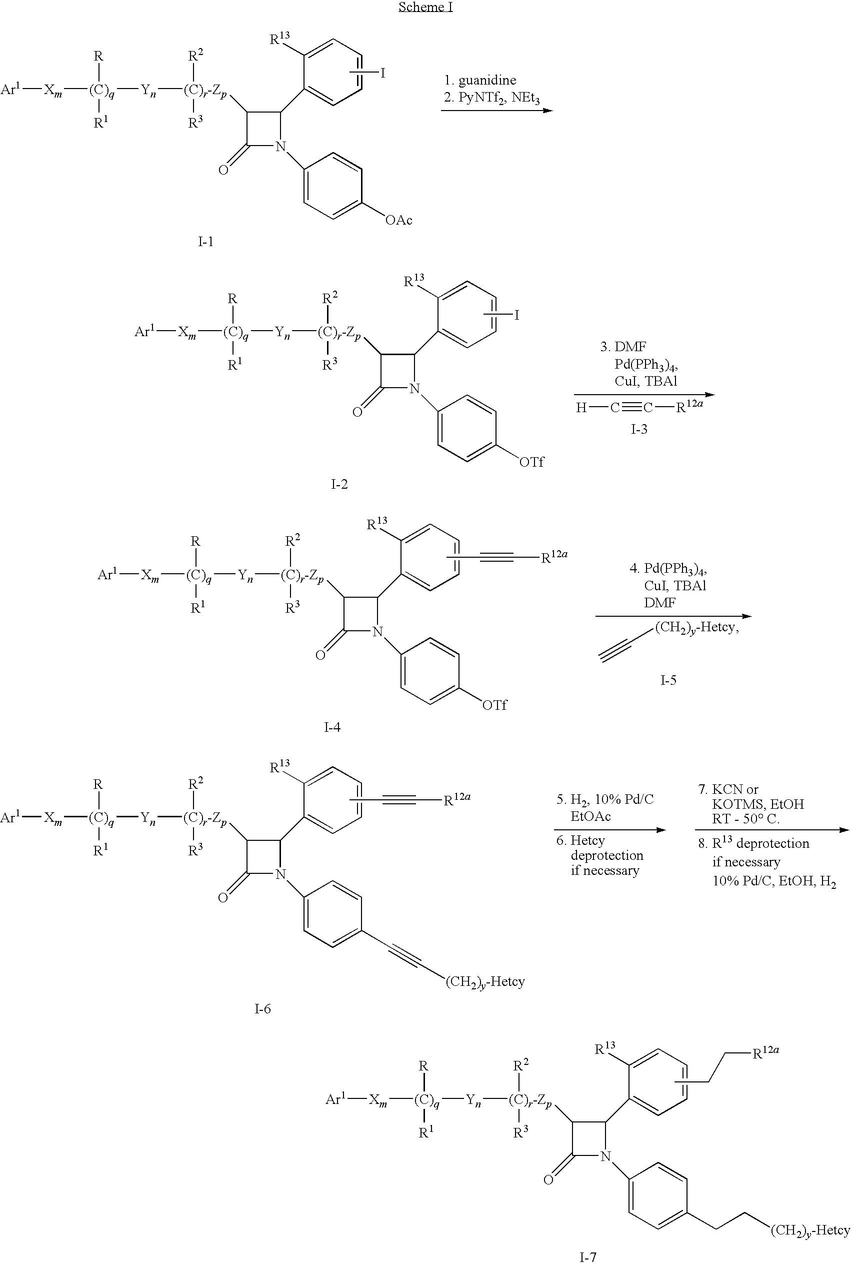

In an alternative procedure shown in Scheme II, intermediate I-4 from the above Scheme I may be utilized in reaction using trimethylsilyl acetylene I-8 in the presence of a suitable palladium catalyst such as tetrakistriphenylphosphine palladium(0) and copper(I) iodide with an initiator such as tetrabutylammonium iodide. The reaction is usually performed in an inert organic solvent such as DMF, at RT to 50° C., for a period of 1 to 5 hrs. The intermediate possessing a trimethylsilylalkynyl group may subsequently be treated with tetra-n-butylammonium fluoride in THF at 0° C. to remove the TMS-group and afford the terminal alkyne of structure I-9. This intermediate may be utilized in a second cross coupling reaction with a heteroaryl-X compound wherein X=Br, I, or OTf in the presence of a suitable palladium catalyst such as tetrakistriphenylphosphine palladium(0) and copper(I) iodide with an initiator such as tetrabutylammonium iodide. The reaction is usually performed in an inert organic solvent such as DMF, at RT to 50° C., for a period of 1 to 5 hrs, and the product possesses an alkynyl-heteroaryl group of structure I-10. Similar reaction steps as described in Scheme I may be utilized as outlined in Scheme II to afford compounds of the present invention I-7. For example, hydrogenation of this bisalkyne intermediate I-10, an additional deprotection step may be included if there are useful protecting groups on the heteroaryl group know to those skilled in the art necessary to allow the chemistry to proceed in a facile fashion. Hydrolysis or cleavage of any remaining hydroxyl protecting groups may be achieved with potassium cyanide or potassium trimethylsilanoate in an alcohol solvent such as ethanol at ambient temperature or heated to 50° C. for 1-2 hours affords the free hydroxyl groups of compounds I-7. When R¹³ is the 2-benzyloxy substituent, a second deprotection step using 10% palladium on carbon in ethanol under hydrogen atmosphere is required as a final deprotection to afford the 2-hydroxy substituted phenyl as in the structure of type I-7.

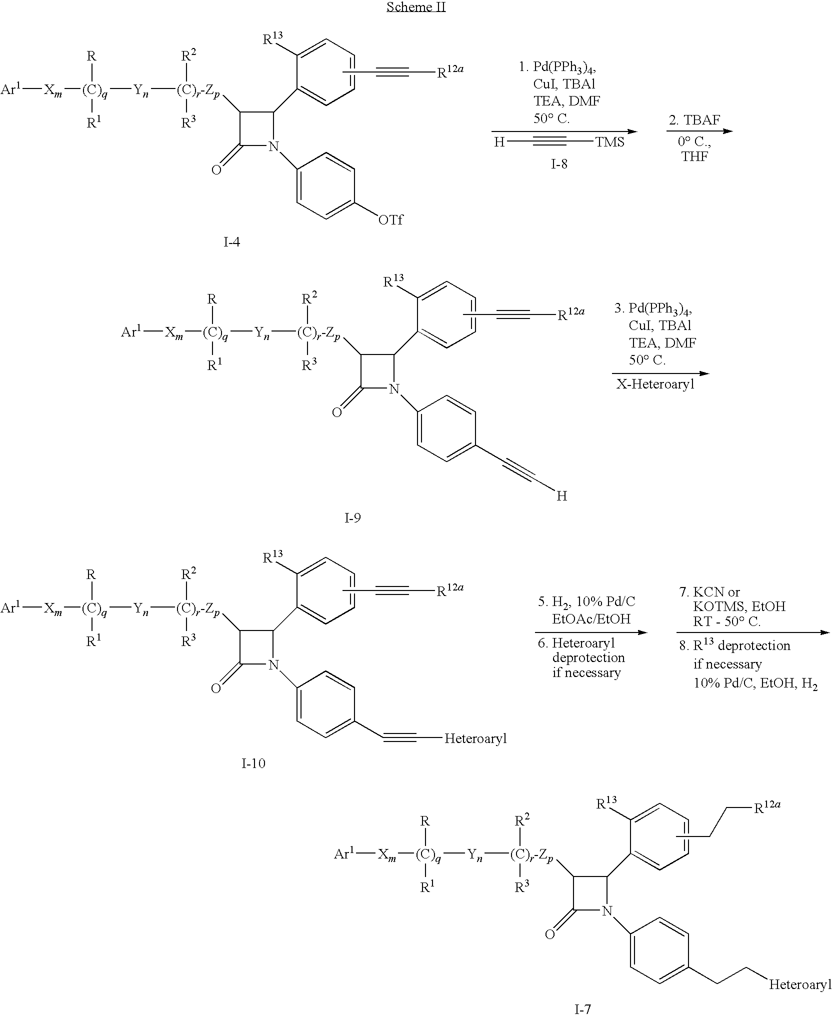

A third synthesis route to compounds of the present invention is outlined in Scheme III. Cross-coupling of iodide intermediate I-1 with allyl or vinyl stannane intermediates (y=0, 1) may be performed in the presence of a palladium catalysts such as Pd(PPh₃)₄ or PdCl₂(PPh₃)₂ in an inert solvent such as DMF at RT or elevated temperature. The subsequent vinyl compound I-11 may be reacted in an olefin cross metathesis with a vinyl intermediate containing R^{12a} using an appropriate catalyst useful olefin metathesis known to those skilled in the art. These catalysts may include the “Shrock” catalyst or the “Zhan” catalyst to produce the intermediates of general structure I-12. The acetoxy group may be converted to the triflate using procedures described above to produce I-13 which may undergo aklyne cross coupling with TMS-acetylene, silicon removal and then a second cross-coupling with heteroaryl-X groups as described in earlier the Schemes to arrive at intermediate I-14. The intermediate I-14 may be converted to compounds of the present invention I-7 by the previously described hydrogenation and subsequent deprotection steps necessary to complete the synthesis.

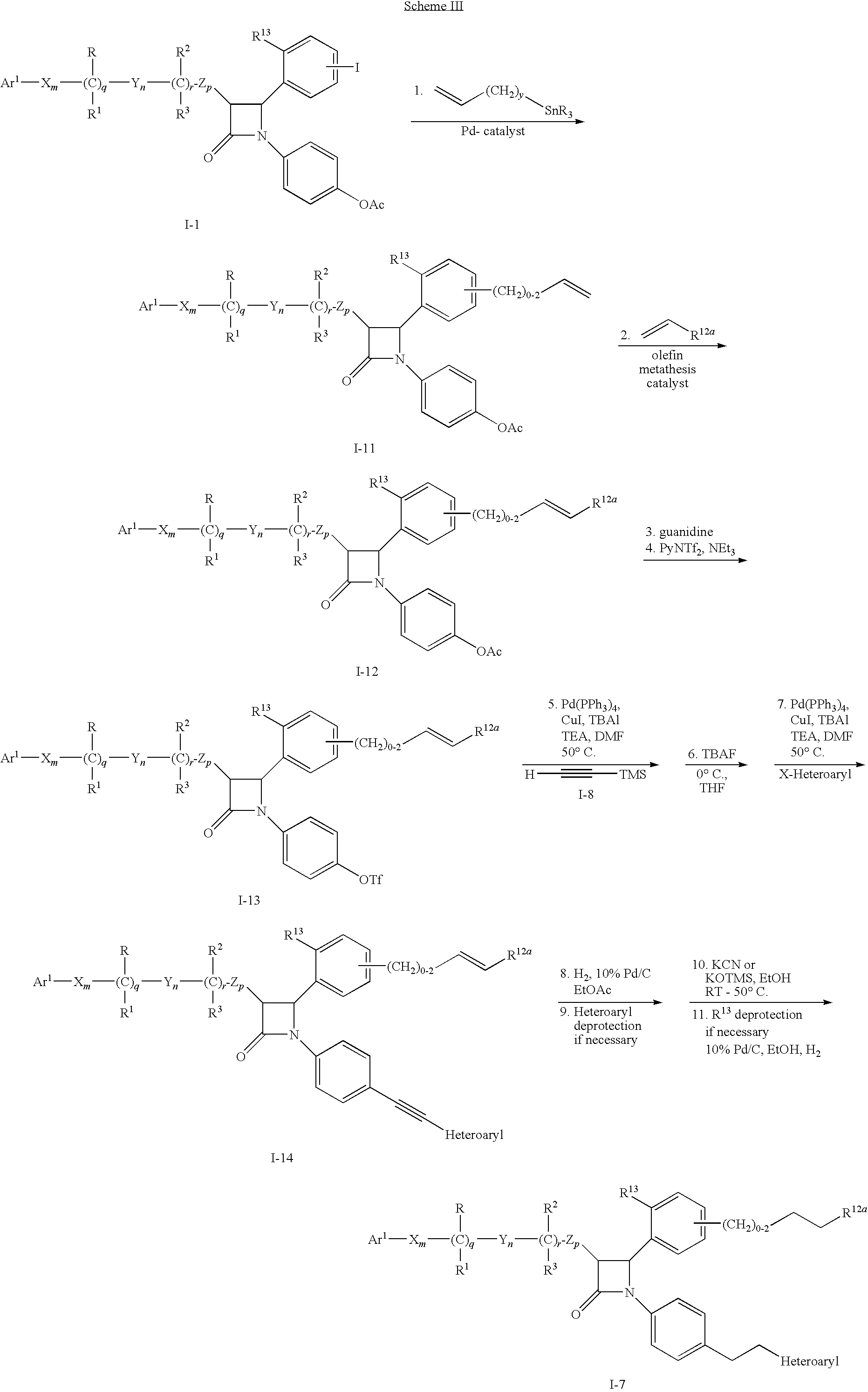

Scheme IV describes the synthesis of compounds of present invention that contain heteroatom linked heteroaryl groups at R⁹ of the present invention. The intermediate 1-4 may be reacted in a Pd-catalyzed cross-coupling reaction using the general conditions described earlier with an alkynylalcohol of general structure I-15. Alternatively the hydroxyl group of I-15 may be protected. The resulting alcohol intermediate I-16 may be hydrogenated using the general conditions described above and the resulting alcohol oxidized to an aldehyde using conditions known to those skilled in the art such as the “Dess-Martin” reagent to provide intermediate I-17. The aldehyde group of I-17 may be reacted in a reductive amination reaction with alkyl, cyclic alkyl/heteroalkyl, aryl or heteroaryl amine compounds using conditions known to those skilled in the art such as sodiumtriacetoxyborohydride in the presence of a buffer such as KOAc and molecular sieves. The reaction product so obtained may be deprotected using the general procedures described earlier to produce compounds of the present invention I-18 in which a nitrogen atom is in the link from the aryl group to the alkyl, cyclic alkyl/heteroalkyl, aryl or heteroaryl group.

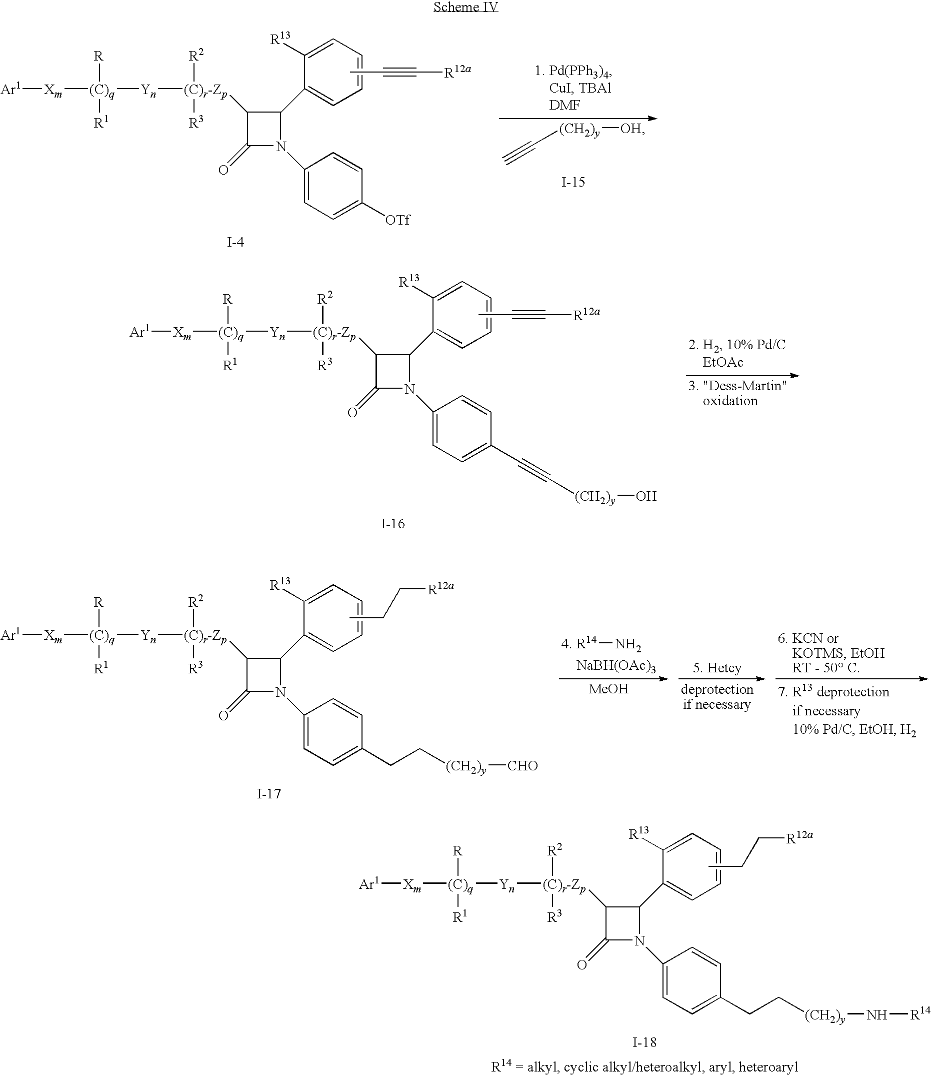

In a related approach, compounds of the general invention that contain oxygen linked heteroaryl groups at R⁹ may be prepared as outlined ion Scheme V. The intermediate I-19 may be prepared as a result of the above mentioned cross-coupling reaction of intermediate I-4 with alkynyl alcohols I-15 (or protected variants thereof) followed by hydrogenation under the usual conditions. The alcohol intermediate I-19 may be reacted in an ether formation reaction with alkyl-, cyclic alkyl/heteralkyl-, aryl- or heteroaryl-OH compounds or related tautomers using the conditions such as triphenyl phosphine and diethylazodicarboxylate. The desired product may then undergo the subsequent deprotections steps described earlier to obtain compounds of the present invention I-20 that contain an oxygen atom in the link from the aryl group to the alkyl, cyclic alkyl/heteroalkyl, aryl or heteroaryl group.

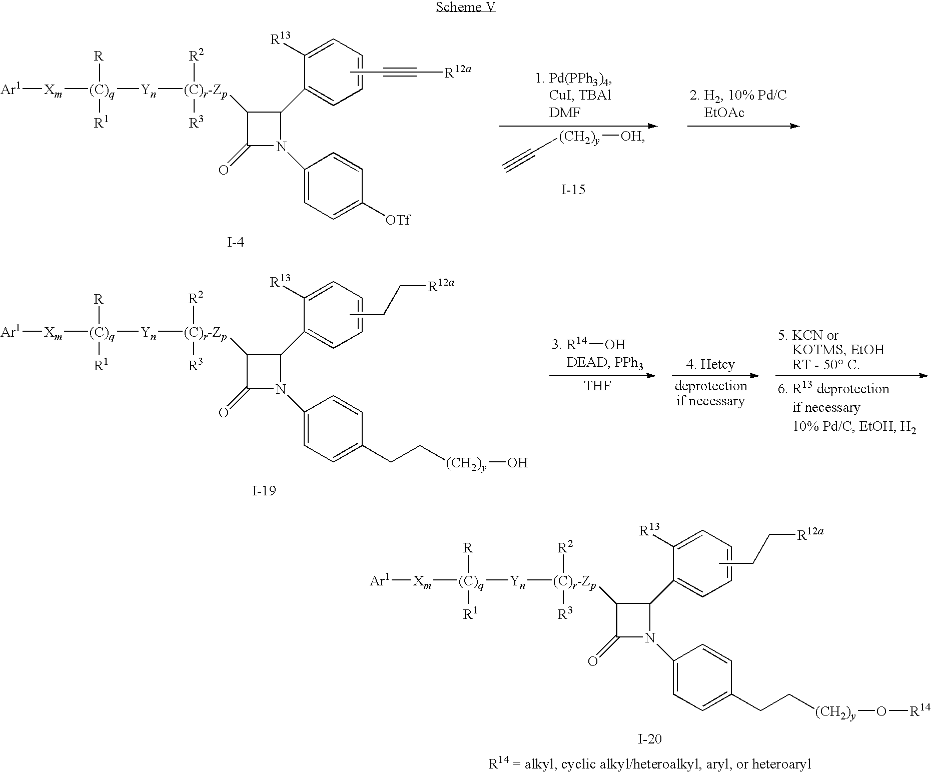

Scheme VI describes the preparation of compounds of the present invention in which alcohol groups are contained on the linking group from the aryl group to the R^{12a} group. The olefin of the intermediate I-12 from the above Scheme III may be reacted in a dihydroxylation reaction using conditions known to those skilled in the art such as catalytic osmiumtetroxide and N-methylmorpholine N-oxide to produce diol compounds I-21 in which R═H. Alternatively, the subsequent diols may be protected as necessary to accommodate subsequent chemistry so the reaction sequence proceeds to the desired compounds. The resulting intermediate I-21 may be processed using reactions similar to those described in the above Schemes to produce intermediates I-22, I-23 and after appropriate hydrogenation and subsequent deprotection steps to prepare compounds of the present invention of general structure I-24.

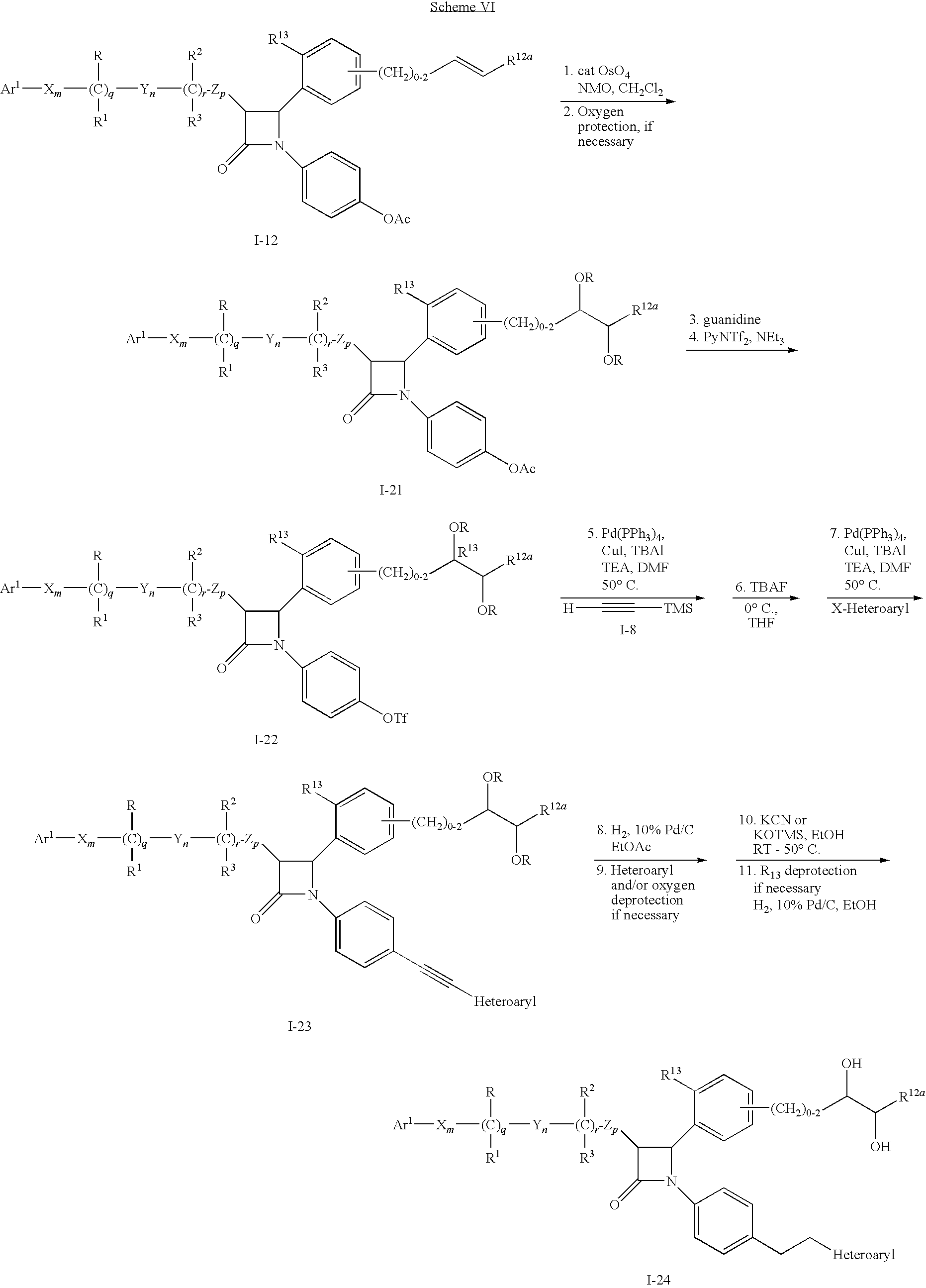

Scheme VII describes the preparation of compounds of the present invention in which the heterocycle is substituted directly onto the phenyl moiety. Conversion of I-4 to the boron pincolate ester (I-26) can be achieved by treatment with dichloro[1,1′-bis(diphenylphosphino)ferrocene]palladium(II) and Bis(pinacolato)diboron in dioxane in the presense of a mild base such as potassium acetate heated to 60° C. overnight. The resulting boronate ester I-27 is treated with a halogenated (preferably I, Br) aryl or heteroaryl moiety of type I-27 in the presence of a suitable palladium catalyst such as dichloro[1,1′-bis(diphenylphosphino)ferrocene]palladium(II) and a mild organic base such as triethylamine. The reaction is usually performed in an inert organic solvent such as DMF, at 40° C. 33 to 65° C., for a period of 1 to 8 hrs, and the product possesses the aryl or heteroaryl substituent directly incorporated onto the phenyl as seen in structure I-28. Hydrogenation of this alkyne intermediate I-28 by treatment with 10% palladium on carbon catalyst under hydrogen atmosphere in a solvent such as ethyl acetate over 15-24 hours may achieve hydrogenation of the triple bond along with the removal of any benzyl protecting groups in I-28. An additional deprotection step may be included if there are useful protecting groups on the heteroaryl group know to those skilled in the art necessary to allow the chemistry to proceed in a facile fashion. These protecting groups may include trityl groups, t-butylcarbamate groups or other groups suitable for the protection of heterocyclic compounds or the functional groups attached to the heterocyclic group known to those skilled in the art. Hydrolysis or cleavage of any remaining hydroxyl protecting groups may be performed at this time, or non-benzylic protecting groups can be removed prior to the hydrogenation step. For example, diols protected as acetals that are contained in R^{12a} may be removed by treatment with aqueous acid. When R^{12a} contains one or more acetate groups, deprotection with potassium cyanide or potassium trimethylsilanoate in an alcohol solvent such as ethanol at ambient temperature or heated to 50° C. for 1-2 hours affords the free hydroxyl groups to form compounds of the present invention I-29. When R¹³ is the 2-benzyloxy substituent, a second deprotection step using 10% palladium on carbon in ethanol under hydrogen atmosphere is required as a final deprotection to afford the 2-hydroxy substituted phenyl as in the structure of type I-29.

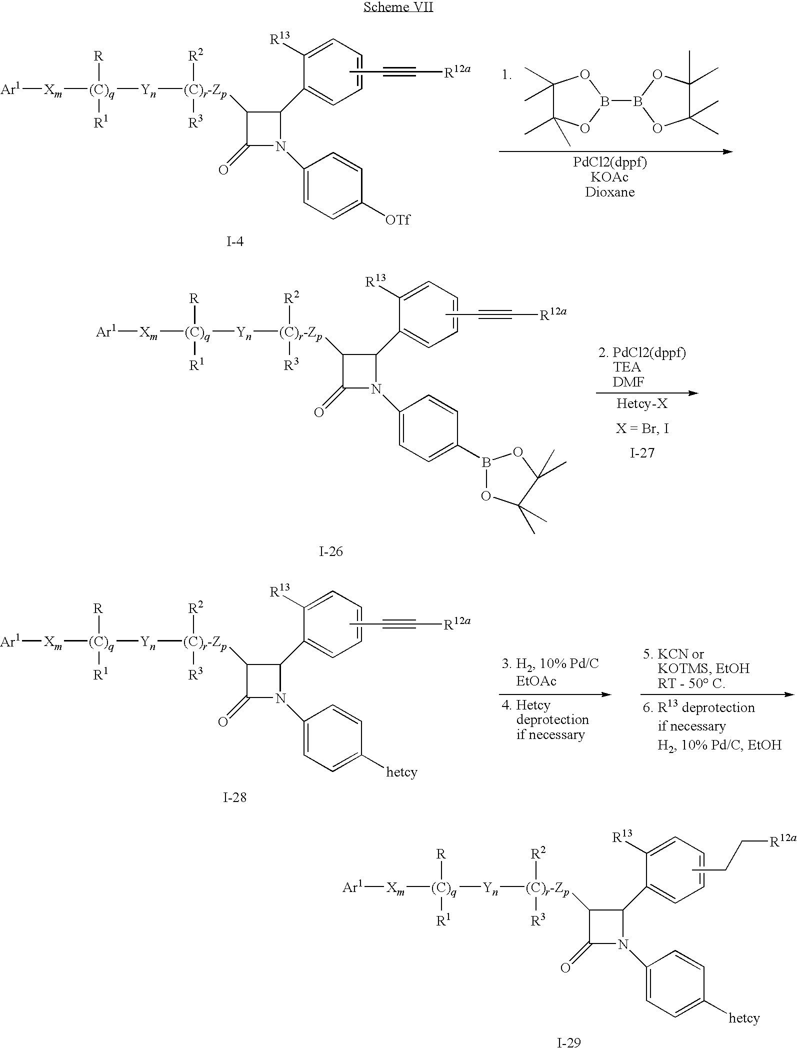

In an alternative approach, compounds of the same general invention may be prepared as outlined in Scheme VIIb. In this scheme the aryl or heteroaryl moiety possesses the boronic acid and the beta-lactam core structure contains the 4-substituted halogen on the N-linked phenyl group. The iodo-phenyl intermediate of the structure I-30 is treated with the boronic acid of the type I-31 in the presence of a suitable palladium catalyst such as dichloro[1,1′-bis(diphenylphosphino)ferrocene]palladium(II) and a mild organic base such as triethylamine. The acetate can be converted to the triflate described previously in the prior schemes. The resulting triflate I-33 is treated with a terminal alkyne of type I-2 containing the R^{12a} group in the presence of a suitable palladium catalyst such as tetrakistriphenylphosphine palladium(0) or [1,1′-bis(diphenylphosphino)ferrocene]dichloropalladium(II) or the like, and copper(I) iodide and an initiator such as tetra-n-butylammonium iodide. The reaction is usually performed in an inert organic solvent such as DMF, between room temperature and 100° C., for a period of 6-48 h, and the product is an internal alkyne of structural formula I-34. Then following the same procedures as described in Scheme VII, the compounds of structure I-28 may be synthesized.

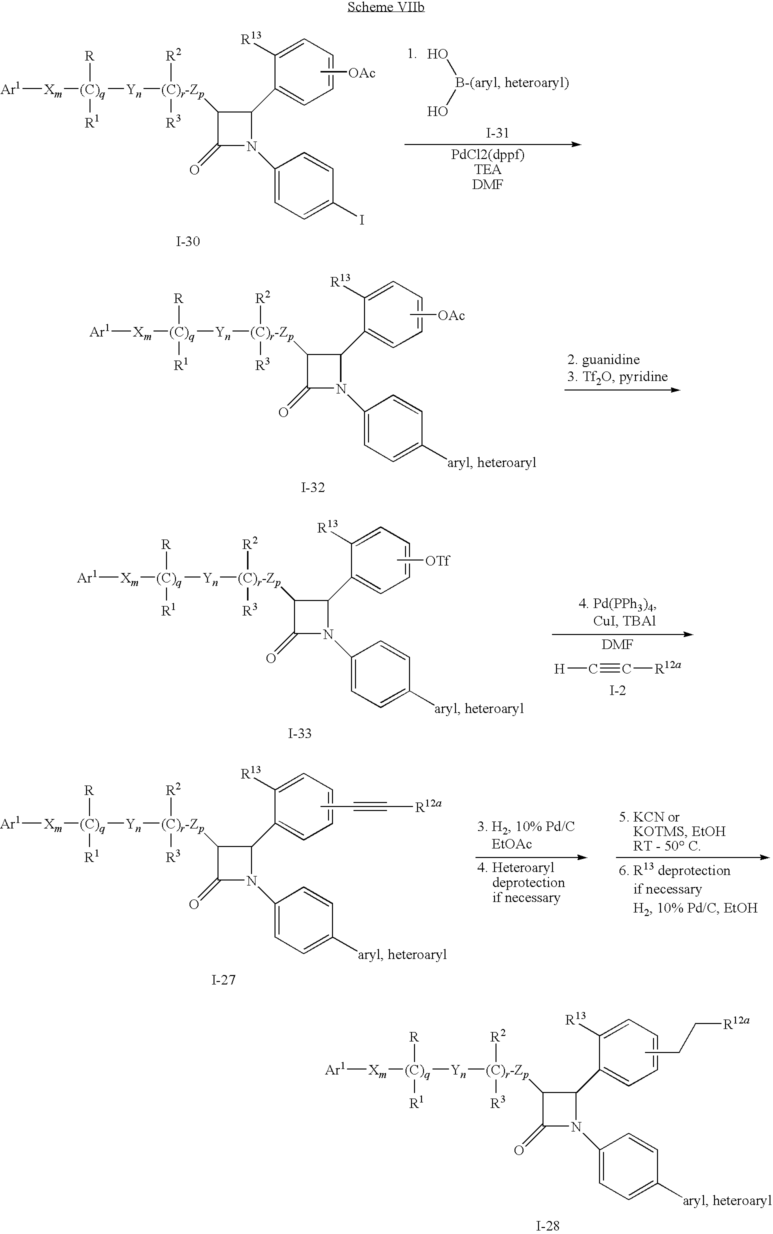

In a related approach, compounds of the general invention, I-38 containing the methylene tether between the phenyl and heterocycle, may be prepared as outlined in Scheme VIII. In this scheme, the benzylic boronic acid of the aryl or heteroaryl moiety of the type I-34 may be prepared for the Suzuki cross coupling of the iodo intermediate I-30. The iodo-phenyl intermediate of the structure I-30 may be treated with the boronic acid of the type I-34 in the presence of a suitable palladium catalyst such as dichloro[1,1′-bis(diphenylphosphino)ferrocene]palladium(II) and a mild organic base such as triethylamine. The acetate may then be converted to the triflate as described previously in the above schemes. The triflate I-36 may then be treated with a terminal alkyne of type I-2 containing the R^{12a} group in the presence of a suitable palladium catalyst such as tetrakistriphenylphosphine palladium(0) or [1,1′-bis(diphenylphosphino)ferrocene]dichloropalladium(II) or the like, and copper(I) iodide and an initiator such as tetra-n-butylammonium iodide. The reaction may be performed in an inert organic solvent such as DMF, between room temperature and 100° C., for a period of 6-48 h, and the product should contain an internal alkyne of structural formula I-37. Then, following the same procedures as described in Scheme VII, the following compounds of structure I-38 may be synthesized.

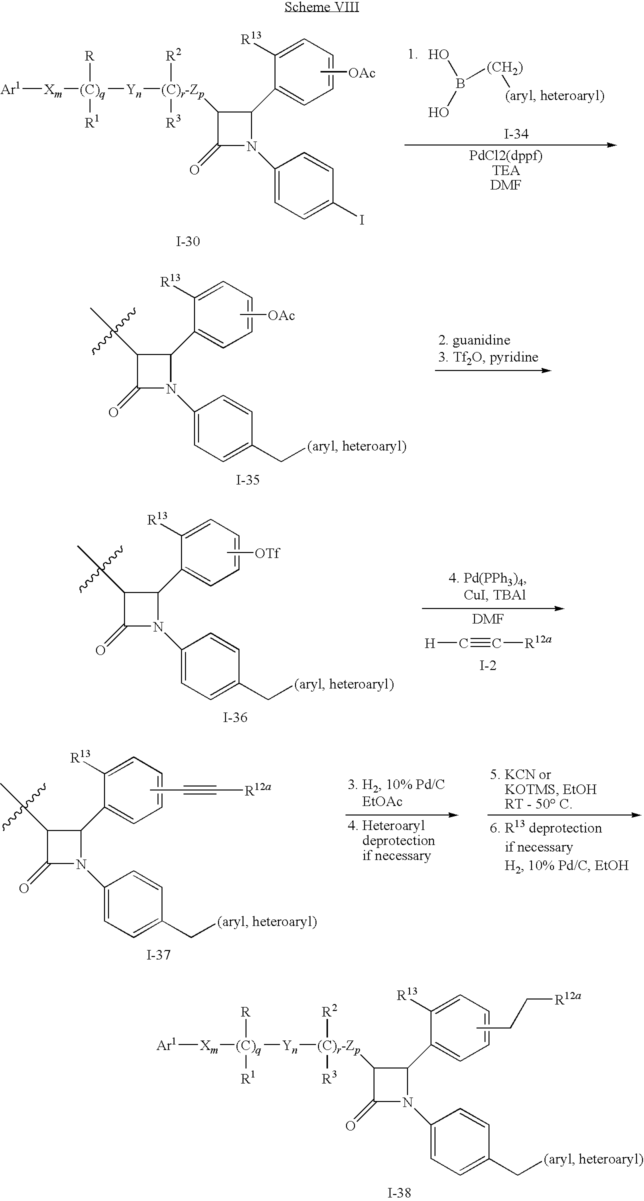

### Preparation of Intermediates

### Preparation of 5-ethynyl-2,2-dimethyl-1,3-dioxan-5-yl acetate (i-1)

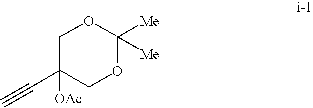

To a dry 250 mL roundbottom flask was charged with a 0.5M solution of ethynylmagnesium bromide in THF (115 mL, 57.7 mmol) under nitrogen atmosphere. The resulting solution was cooled to 0° C. in an ice bath. To the cooled solution was added slowly a solution of 2,2-dimethyl-1,3-dioxane-5-one (5 g, 38.44 mmol) in 50 mL dry THF. The ice bath was removed and the resulting reaction mixture was stirred at ambient temperature for 1.5 hrs. The reaction mixture was quenched with sat. aq. NH₄Cl (50 mL) and then extracted with ethyl acetate (100 mL). The organic layer was dried over Na₂SO₄, filtered and the solvent removed under vacuum to afford the crude intermediate.

The crude intermediate was dissolved in CH₂Cl₂ (100 mL) under nitrogen atmosphere. To the resulting solution was added simultaneously by syringe acetic anhydride (4.34 mL, 46 mmol) and TEA (6.4 mL, 46 mmol). To the reaction mixture was added DMAP (0.56 g, 4.6 mmol). The reaction mixture was stirred for 3 hrs at room temperature at which time the reaction was quenched by the addition of 1N aq. HCl (100 mL). The reaction mixture was transferred to separatory funnel and the organic layer was separated. The organic layer was washed with aq. NaHCO₃ (100 mL), water (50 mL), brine, dried, filtered and the solvent removed under vacuum to afford the title compound (i-1) which was used without further purification. ¹HNMR (500 MHz, CDCl₃) δ: 4.14 (d, J=12.6, 2H) 4.07 (d, J=12.6 Hz, 2H), 2.65 (s, 1H), 2.12 (s, 3H), 1.45 (s, 3H), 1.41 (s, 3H).

### Preparation of 2-ethynylpropane-1,2,3-triol 1,3-diacetate (i-2)

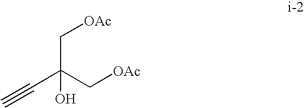

To a cooled solution, 0° C., of 2-oxopropane-1,3-diyl diacetate (17.5 g, 100 mmol) in anhydrous THF (50 mL) under nitrogen atmosphere was added dropwise via syringe 0.5M ethynylmagnesium bromide (200 mL) and the resulting solution stirred for 3 hours allowing to warm to room temperature. The mixture was quenched with a saturated solution of ammonium chloride (50 mL) and extracted with 200 mL ethyl acetate. The organics were dried over magnesium sulfate, filtered, and evaporated under vacuum. MPLC purification with a gradient eluant of 10-50% ethyl acetate in hexane afforded the title compound. ¹H-NMR (400 MHz, CDCl₃) δ: 4.28 (d, J=11.5 Hz, 2H), 4.22 (J=11.5 Hz, 2H), 3.26 (s, 1H), 2.55 (s, 1H), 2.13 (s, 6H).

The compounds (3R,4S)-3-[(3S)-3-(4-fluorophenyl)-3-hydroxypropyl]-4-(4-hydroxyphenyl)-1-(4-iodophenyl)azetidin-2-one (i-3) and (i-4) were prepared according to Burnett, D. S.; Caplen, M. A.; Domalski, M. S.; Browne, M. E.; Davis, H. R. Jr.; Clader, J. W. Bioorg. Med. Chem. Lett. (2002), 12, 311. Compound i-5 is the dihydroxy-protected analog of i-4, where the protecting groups are acetyl.

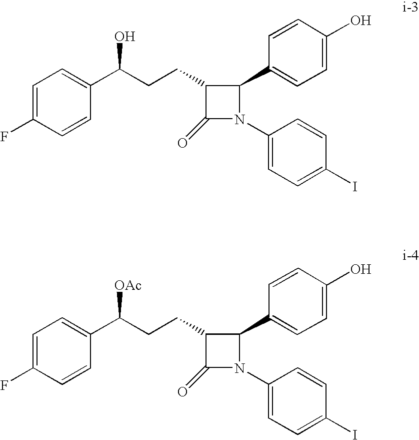

### Preparation of 4-[(2S,3R)-3-[(3S)-3-(acetyloxy)-3-(4-fluorophenyl)propyl]-1-(4-iodophenyl)-4-oxoazetidin-2-yl]phenyl acetate (i-5)

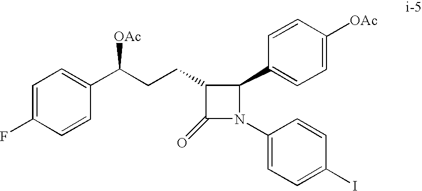

To a solution of (1S)-1-(4-fluorophenyl)-3-[(2S,3R)-2-(4-hydroxyphenyl)-1-(4-iodophenyl)-4-oxoazetidin-3-yl]propyl acetate (i-4) (2 g, 3.58 mmol) (prepared according to Burnett, D. S.; Caplen, M. A.; Domalski, M. S.; Browne, M. E.; Davis, H. R. Jr.; Clader, J. W. Bioorg. Med. Chem. Lett. (2002), 12, 311) in CH₂Cl₂ (25 mL) under nitrogen atmosphere was added acetic anhydride (0.4 mL, 4.30 mmol), triethylamine (0.75 mL, 5.38 mmol) and DMAP. The reaction mixture was stirred at RT for 1 hr and the solvent removed under vacuum. The residue was purified by MPLC (silica column) with stepwise gradient elution; (0-100% EtOAc/hexanes as eluent) to afford the title compound (i-5). m/z (ES) (M-OAc)⁺. ¹HNMR (500 MHz, CDCl₃) δ: 7.57 (d, J=8.6, 1H) 7.38-7.26 (m, 5H), 7.22 (br d, J=7.1H, 2H), 7.14 (d, J=8.5 Hz, 1H), 7.08-7.02 (m, 3H), 5.74 (t, J=6.7 Hz, 1H), 4.62 (d, J=2.3 Hz, 1H), 3.10 (dt, J=2.3, 7.8 Hz, 1H), 2.34 (s, 3H), 2.08 (s, 3H), 2.09-2.03 (m, 2H), 1.94-1.86 (m, 2H). (1S)-3-[(2S,3R)-2-[2,4-bis(benzyloxy)phenyl]-1-(4-iodophenyl)-4-oxoazetidin-3-yl]-1-(4-fluorophenyl)propyl acetate (i-6) was prepared from 2,4-bisbenzyloxyacetaldehyde and 4-iodoaniline using procedures as described in Vaccaro, W. D. et al., Bioorg. Med. Chem., vol. 6 (1998), 1429-1437.

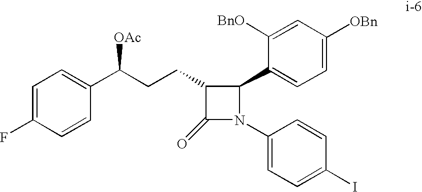

The above intermediates i-3, i-4, and i-5 may utilized in procedures similar to those described in the above Schemes in which the order of introduction of side chains on the aryl groups of the azetidinone ring is reversed.

### Preparation of 1-prop-2-yn-1-yl-1H-1,2,4-triazole (i-7)

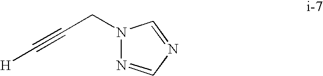

To a solution of 1H-1,2,4-triazole (5 g, 72.4 mmol) in ethanol (50 mL) cooled in a ice-bath was added solution of NaOH (2.9 g, 74.7 mmol) in 5 mL water which immediately resulted in the formation of a white precipitate. To the resulting mixture was added dropwise over 1 h propargyl bromide (8.2 mL, 74.7 mmol). After completion of the addition, the reaction mixture was allowed to warm to RT and stirred for 48 hr. Water (100 mL) was added and the reaction mixture was transferred to a separatory funnel and extracted with methylene chloride (3×75 mL). The combined organic layers were washed with water (2×), dried over Na₂SO₄ filtered and the solvent removed under vacuum. The residue was purified by column chromatography on silica gel eluting with 2% MeOH in CH₂Cl₂ to provide of the title compound. ¹H NMR (500 MHz, CDCl₃) δ: 8.29 (s, 1H), 7.96 (s, 1H), 4.99 (d, J=2.7, 2H), 2.60 (t, J=2.7, 1H)

3-Iodo-1-trityl-1H-1,2,4-triazole (i-8) was prepared according to the procedure described in PCT publication WO 93/15610 A1, (see Examples 1, 4 and 5 therein). ¹HNMR (500 MHz, CDCl₃) δ: 8.09 (s, 1H), 7.38 (m, 9H), 7.04 (m, 6H).

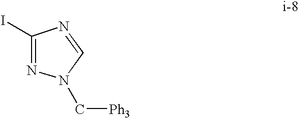

### Preparation of 3-(1-trimethylsilylethyn-2-yl)-1-trityl-1H-1,2,4-triazole (i-9)

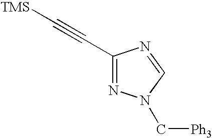

Nitrogen gas was bubbled through a solution of 3-iodo-1-trityl-1H-1,2,4-triazole (37.3 g, 85.35 mmol), and triethylamine (17.8 ml, 128 mmol) in anhydrous DMF (300 ml) heated at 35° C. for 30 mins. Pd(PPh₃)₂Cl₂ (2.4 g, 3.4 mmol) and CuI (651 mg, 3.4 mmol) were added followed by addition of ethynyltrimethylsilane (18 ml, 128 mmol) in anhydrous DMF (18 ml) over 15 hours via syringe pump. After complete addition the mixture was heated at 35° C. for a further 5 hours. The mixture was poured into water (700 ml) and extracted with EtOAc (3×300 ml). Combined EtOAc layers washed with water (2×500 ml), sat. NaCl (250 ml), dried over Na₂SO₄, filtered and evaporated. The residue was purified by MPLC on silica gel eluting with a gradient from 100% hexanes to 10% EtOAc in hexanes to afford the title compound. ¹HNMR (500 MHz, CDCl₃) δ: 7.96 (s, 1H), 7.37 (m, 9H), 7.14 (m, 6H), 0.27 (s, 9H).

### Preparation of 3-ethynyl-1-trityl-1H-1,2,4-triazole (i-10)

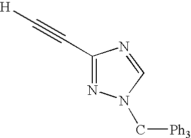

Tetrabutylammonium fluoride (3.8 ml of a 1.0M solution in THF, 3.8 mmol) was added to a solution of 3-(1-trimethylsilylethyn-2-yl)-1-trityl-1H-1,2,4-triazole (7.75 g, 19 mmol) in anhydrous THF (50 ml), and the resulting mixture stirred for 30 mins. Evaporated to dryness, and the residue partitioned between CH₂Cl₂ and water. The organic layer was washed with sat. NaCl, dried over Na₂SO₄, filtered and evaporated. The residue was triturated with Et₂O/hexanes to afford of the title compound. ¹HNMR (500 MHz, CDCl₃) δ: 7.99 (s, 1H), 7.38 (m, 9H), 7.15 (m, 6H), 3.10 (s, 1H).

### Preparation of 1-prop-2-yn-1-yl-1H-1,2,3-triazole (i-11)

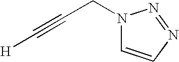

The title compound was prepared from 1H-1,2,3-triazole according to the procedure for intermediate (i-7). NMR (500 MHz, CDCl₃) δ: 7.80 (s, 1H), 7.74 (s, 1H), 5.22 (d, J=2.5, 2H), 2.59 (t, J=2.5, 1H)

### Preparation of 2-bromothiazole-4-carboxamide (i-12)

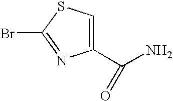

A mixture of ethyl-2-bromothiazole-4-carboxylate (2.95 g, 12.5 mmol) and 7N ammonia in methanol solution (40 ml, 280 mmol) contained within a sealed tube was warmed at 50° C. for 15 hours. The mixture was cooled and evaporated. The residue was triturated with Et₂O/hexanes, filtered and dried to give the title compound. ¹HNMR (500 MHz, DMSO-d6) δ: 8.27 (s, 1H), 7.83 (br s, 1H), 7.64 (br s, 1H).

### Preparation of 2-bromothiazole-5-carboxamide (i-13)

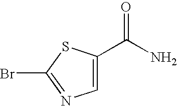

The title compound was prepared from methyl-2-bromothiazole-5-carboxylate according to the procedure for intermediate (i-12). ¹HNMR (500 MHz, DMSO-d6) δ: 8.19 (br s, 2H), 7.76 (br s, 1H).

### Preparation of 4-bromothiazole-2-carboxylic acid (i-14)

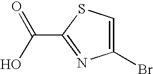

A solution of 2,4-dibromothiazole (5 g, 20.6 mmol) in anhydrous Et₂O (30 ml) was added in a dropwise manner to a solution of butyl lithium (9.9 ml of a 2.5M solution in hexanes, 24.7 mmol) in anhydrous Et₂O (70 ml) cooled at −78° C., at such a rate that the temperature did not rise above at −73° C. After addition was complete the mixture was stirred at −78° C. for 1 hour. Carbon dioxide gas was bubbled through the mixture for 5 mins than a pellet (˜5 g) of solid carbon dioxide added and the mixture allowed to warm to room temperature. Water (100 ml) added and the aqueous layer extracted further with Et₂O. The aqueous layer was acidified with conc. HCl and extracted with Et₂O (3×100 ml), combined Et₂O layers dried over Na₂SO₄, filtered and evaporated. The residue was crystallized from Et₂O/Hexanes to give of the title compound. ¹HNMR (500 MHz, DMSO-d6) δ: 8.20 (s, 1H).

### Preparation of methyl-4-bromothiazole-2-carboxylate (i-15)

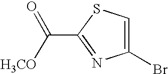

Intermediate 14 (1.98 g, 9.5 mmol) was dissolved in methanol (50 ml) and N-(3-dimethylaminopropyl)-N′-ethylcarbodiimide hydrochloride (2.73 g, 14.3 mmol), 1-hydroxybenzotriazole (1.93 g, 14.3 mmol), and diisopropylethylamine (2.5 ml, 14.3 mmol) added. The resulting mixture was stirred at room temperature for 17 hours. The mixture was evaporated, and the resulting residue partitioned between CH₂Cl₂ and water. The organic layer was washed with 1N HCl, sat. NaHCO₃, sat. NaCl, dried over Na₂SO₄, filtered and evaporated to give the title compound. ¹HNMR (500 MHz, CDCl₃) δ: 7.57 (s, 1H), 4.04 (s, 3H).

### Preparation of 4-bromothiazole-2-carboxamide (i-16)

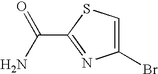

The title compound was prepared from methyl-4-bromothiazole-2-carboxylate according to the procedure for intermediate (i-12). ¹HNMR (500 MHz, CDCl₃) δ: 7.54 (s, 1H), 7.12 (br s, 1H), 5.92 (br s, 1H).

### Preparation of 5-(trimethylstannyl)thiazole-2-carboxamide (i-17)

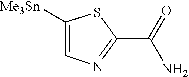

Lithium bis(trimethylsilylamide) (86 ml of a 1M solution in THF, 86 mmol) was added to a solution of thiazole-2-carboxamide (2.2 g, 17.2 mmol) and trimethyltin chloride (5.14 g, 25.8 mmol) in anhydrous THF (80 ml) cooled at −40° C. After addition was complete the mixture was warmed to −20° C. and stirred at this temperature for 7 hours. Quenched by the addition of sat. NH₄Cl (200 ml) and EtOAc (250 ml). Organic layer separated, washed with sat. NaCl, dried over Na₂SO₄, filtered and evaporated. The residue was purified by MPLC on silica eluting with a gradient of 100% hexanes to 40% EtOAc in hexanes to give the title compound. ¹HNMR (500 MHz, CDCl₃) δ: 7.82 (s, 1H), 7.24 (br s, 1H), 6.24 (br s, 1H), 0.47 (t, J=28.8, 9H).

### Preparation of 5-iodothiazole-2-carboxamide (i-18)

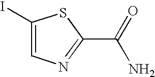

To a solution of intermediate 17 (1.5 g, 5.17 mmol) in anhydrous THF (25 ml) cooled at −55° C. was added N-iodosuccinamide (1.16 g, 5.17 mmol), mixture stirred at this temperature for 10 mins. then allowed to warm to room temperature and stirred for 30 mins. Chloroform (50 ml) added and the mixture washed with sat. NaCl (3×70 ml), dried over Na₂SO₄, filtered and evaporated. The residue was triturated with hexanes, filtered and dried to give the title compound. ¹HNMR (500 MHz, DMSO-d6) δ: 8.16 (br s, 1H), 8.09 (s, 1H), 7.91 (br s, 1H).

### Preparation of methyl-5-bromothiazole-4-carboxylate (i-19)

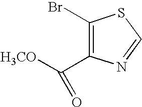

The title compound was prepared 4-bromothiazole-2-carboxylate according to the procedure for intermediate (i-15). ¹HNMR (500 MHz, CDCl₃) δ: 8.81 (s, 1H), 4.00 (s, 3H).

### Preparation of 5-bromothiazole-4-carboxamide (i-20)

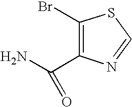

The title compound was prepared from 4-bromothiazole-2-carboxylate according to the procedure for intermediate (i-12) 700 mg. ¹HNMR (500 MHz, DMSO-d6) δ: 9.14 (s, 1H), 7.81 (br s, 1H), 7.64 (br s, 1H).

### Preparation of ethyl 2-amino-5-methylthiazole-4-carboxylate (i-21)

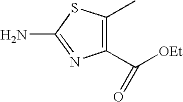

N-bromosuccinamide (36.77 g, 206 mmol) was added to a solution of ethyl-2-hydroxybutyrate (13.65 g, 103 mmol) in carbon tetrachloride (200 ml), and the resulting mixture heated at reflux for 5 hours. The mixture was cooled and filtered through celite 545®, and the filtrate evaporated. The residue was taken up in water (129 ml), and thiourea (5.49 g, 72 mmol) added, and the resulting mixture heated to reflux for 15 mins, cooled to room temperature and stirred overnight. The mixture was basified by the addition of NH₄OH and the resulting cream precipitate filtered, washed and washed with further portions of water. The precipitate was taken up in CH₂Cl₂ (500 ml) and EtOH (20 ml), dried over Na₂SO₄, filtered and evaporated to give the title compound. ¹HNMR (500 MHz, CDCl₃) δ: 5.55 (br s, 2H), 4.33 (q, J=7.1, 2H), 2.59 (s, 3H), 1.37 (t, J=7.1, 3H).

### Preparation of Ethyl-2-bromo-5-methylthiazole-4-carboxylate (i-22)

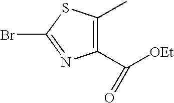

Intermediate 21 (10 g, 53.8 mmol) was added portionwise to a mixture of tert-butyl nitrite (9.58 ml, 80.6 mmol) and copper (II) bromide (18 g, 80.6 mmol) in acetonitrile (200 ml) warmed at 60° C. After complete addition the mixture was heated at 75° C. for 2 hours. The mixture was cooled and poured into 1N HCl (500 ml), and extracted with CH₂Cl₂ (2×200 ml). The combined CH₂Cl₂ extracts were dried over Na₂SO₄, filtered and evaporated to give of the title compound. ¹HNMR (500 MHz, CDCl₃) δ: 4.39 (q, J=7.1, 2H), 2.72 (s, 3H), 1.39 (t, J=7.1, 3H).

### Preparation of 2-bromo-5-methylthiazole-4-carboxamide (i-23)

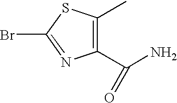

The title compound was prepared from intermediate 22 according to the procedure for intermediate (i-12). ¹HNMR (500 MHz, DMSO-d6) δ: 7.65 (br s, 1H), 7.50 (br s, 1H), 2.68 (s, 3H).

### Preparation of 2-bromothiazole-4-methanol (i-24)

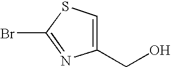

Sodium borohydride (99 mg, 26 mmol) was added portionwise to a solution of 2-bromothiazole-4-carbaldehyde (1 g, 5.2 mmol) in anhydrous methanol (20 ml) cooled in an ice bath. After the addition was complete the cooling was removed and the mixture stirred for 150 mins. The mixture was evaporated and the residue partitioned between 1N HCl (50 ml) and EtOAc (50 ml). the organic layer was washed with sat. NaCl (20 ml), dried over Na₂SO₄, filtered and evaporated to give the title compound. ¹HNMR (500 MHz, CDCl₃) δ: 7.19 (s, 1H), 4.77 (s, 2H), 2.76 (br s, 1H).

### Preparation of 2-bromo-1,3-thiazol-4-yl)methyl methanesulfonate (i-25)

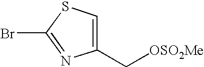

To a solution of intermediate 24 (200 mg, 1 mmol) in anhydrous CH₂Cl₂ (5 ml) cooled at 0° C. was added triethylamine (172 μl, 1.2 mmol) followed by methanesulfonyl chloride (88 μl, 1.1 mmol), and the mixture allowed to warm to room temperature overnight. Diluted with more CH₂Cl₂ (15 ml), washed with water, sat. NaCl, dried over Na₂SO₄, filtered and evaporated. The residue was purified by MPLC on silica gel eluting with a gradient rising from 100% hexanes to 25% EtOAc in hexanes to give the title compound. ¹HNMR (500 MHz, CDCl₃) δ: 7.42 (s, 1H), 5.29 (s, 2H), 3.08 (s, 3H).

### Preparation of 2-bromo-4-methylthiomethyl thiazole (i-26)

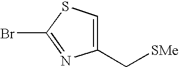

To a solution of intermediate 25 (200 mg, 0.74 mmol) in anhydrous EtOH (3 ml) was added sodium thiomethoxide (57 mg, 0.8 mmol), and the resulting mixture stirred at room temperature for 30 mins. The mixture was evaporated and the residue partitioned between CH₂Cl₂ and water. The organic layer was dried over Na₂SO₄, filtered and evaporated. The residue was purified by MPLC on silica gel eluting with a gradient rising from 100% hexanes to 20% EtOAc in hexanes to give the title compound. ¹HNMR (500 MHz, CDCl₃) δ: 7.10 (s, 1H), 3.79 (s, 2H), 2.12 (s, 3H).

### Preparation of 2-bromo-4-methylsulfonylmethyl thiazole (i-27)

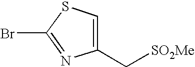

To a solution of intermediate 26 (695 mg, 3.1 mmol) in CH₂Cl₂ (40 ml) cooled in an ice bath was added in one portion 77% 3-chloroperbenzoic acid (1.74 g, 7.75 mmol) and the resulting mixture allowed to warm to room temperature under stirring overnight. Mixture filtered through celite 545®, and the filtrate washed with 1N NaOH (50 ml), dried over Na₂SO₄, filtered and evaporated to give of the title compound. ¹HNMR (500 MHz, CDCl₃) δ: 7.47 (s, 1H), 4.42 (s, 2H), 2.98 (s, 3H).

### Preparation of 2-bromothiazole-5-methanol (i-28)

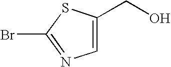

The title compound was prepared from 2-bromothiazole-5-carbaldehyde according to the procedure for intermediate (i-24). ¹HNMR (500 MHz, CDCl₃) δ: 7.40 (s, 1H), 4.82 (s, 2H), 3.00 (br s, 1H).

### Preparation of 2-bromo-5-methylsulfonylmethyl thiazole (i-29)

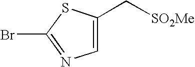

The title compound was prepared from 2-bromothiazole-5-methanol according to the procedures for intermediates (i-25, i-26, i-27). ¹HNMR (500 MHz, CDCl₃) δ: 7.60 (s, 1H), 4.45 (s, 2H), 2.92 (s, 3H).

### Preparation of 5-bromothiazole (i-30)

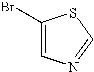

To a solution of 2-amino-5-bromothiazole (12.58 g, 70 mmol) in a mixture of phosphoric acid (106 ml of an 86% solution in water), and conc. nitric acid (19.2 ml) cooled at −5° C. was added over 45 mins a solution of sodium nitrite (7.59 g, 110 mmol) in water (26 ml). After the addition was complete the mixture was stirred at −5° C. for 15 mins, then hypophosphorous acid (38.8 ml) added dropwise over 30 mins keeping the temperature below 0° C. The mixture was stirred at 0° C. for 150 mins then allowed to warm to room temperature overnight. The mixture was poured into a solution of NaOH (85 g) in water (400 ml). 5N NaOH solution added until the mixture reached neutrality, and the resulting mixture extracted with CH₂Cl₂ (3×200 ml). Combined CH₂Cl₂ layers washed with sat. NaCl, dried over Na₂SO₄, filtered and evaporated. The residue was purified by MPLC on silica gel eluting with a gradient rising from 100% hexanes to 10% EtOAc in hexanes to give of the title compound. ¹HNMR (500 MHz, CDCl₃) δ: 8.78 (s, 1H), 7.83 (s, 1H).

### Preparation of ethyl 2-bromo-4-thiazoleacetate (i-31)

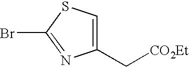

The title compound was prepared from ethyl 2-amino-4-thiazoleaceate according to the procedure for intermediate (i-22). ¹HNMR (500 MHz, CDCl₃) δ: 7.19 (s, 1H), 4.20 (q, J=7.1, 2H), 3.82 (s, 2H), 1.29 (t, J=7.1, 3H).

### Preparation of 2-bromothiazole-4-acetamide (i-32)

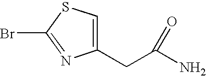

The title compound was prepared from intermediate 31 according to the procedure for intermediate (i-12). ¹HNMR (500 MHz, CDCl₃) δ: 7.15 (s, 1H), 6.47 (br s, 1H), 5.60 (br s, 1H), 3.73 (s, 2H).

### Preparation of 2-(2-bromo-1,3-thiazol-4-yl)ethanol (i-33)

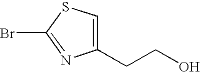

To a solution of intermediate 31 (2.5 g, 10 mmol) in anhydrous Et₂O (40 ml) was added lithium borohydride (381 mg, 17.5 mmol) followed by slow addition of methanol (709 μl, 17.5 mmol), and the resulting mixture stirred for 30 mins. The mixture was cooled in an ice bath and quenched by the addition of 1N HCl (150 ml). The resulting mixture was extracted with CH₂Cl₂ (100 ml), dried over Na₂SO₄, filtered and evaporated. The residue was purified by MPLC on silica gel eluting with a gradient rising from 100% hexanes to 50% EtOAc in hexanes to give of the title compound. ¹HNMR (500 MHz, CDCl₃) δ: 6.98 (s, 1H), 3.94 (t, J=5.9, 2H), 2.98 (t, J=5.9, 2H), 2.59 (s, 1H).

### Preparation of 2-bromo-4-[2-(methylsulfonyl)ethyl]-1,3-thaizole (i-34)

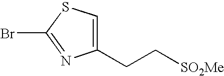

The title compound was prepared from intermediate 32 according to the procedures for intermediates (i-25, i-26, i-27). ¹HNMR (500 MHz, CDCl₃) δ: 7.08 (s, 1H), 3.47 (t, J=8.2, 2H), 3.29 (t, J=8.2), 2.88 (s, 3H).

### Preparation of Ethyl 2-amino-5-[(benzyloxy)methyl]-1,3-thiazole-4-carboxylate (i-35)

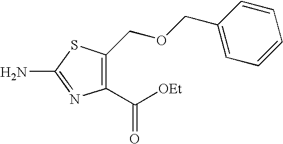

Sodium ethoxide (33.6 ml of a 21% wt solution in ethanol, 90 mmol) was added dropwise to a mixture of benzyloxyacetaldehyde (15 g, 100 mmol), and ethyl dichloroacetate (11.15 ml, 90 mmol) in anhydrous Et₂O (50 ml) cooled at 0° C. The resulting mixture was stirred at 0° C. for 1 hour then more Et₂O (50 ml), and sat. NaCl (100 ml) added. The organic layer was separated dried over Na₂SO₄, filtered and evaporated. The residue was dissolved in ethanol (100 ml) and thiourea (6.55 g, 86 mmol) added, and the resulting mixture heated at reflux for 4 hours. The mixture was cooled and evaporated, and the residue partitioned between water and CH₂Cl₂. The organic layer was extracted with CH₂Cl₂ (×2); the combined CH₂Cl₂ layers washed with water, sat. NaCl, dried over Na₂SO₄, filtered and evaporated. The residue was purified by MPLC on silica gel eluting with a gradient rising from 100% hexanes to 50% EtOAc in hexanes to give the title compound. ¹HNMR (500 MHz, CDCl₃) δ: 7.32 (m, 5H), 5.78 (br s, 2H), 4.99 (s, 2H), 4.63 (s, 2H), 4.33 (q, J=7.1, 2H), 1.36 (t, J=7.13H).

### Preparation of 2-amino-5-[(benzyloxy)methyl]-1,3-thiazole-4-carboxamide (i-36)

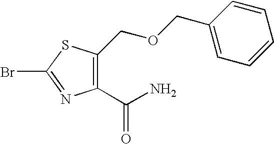

The title compound was prepared from intermediate 35 according to the procedures for intermediates (i-22, and i-12). ¹HNMR (500 MHz, CDCl₃) δ: 7.38 (m, 5H), 7.07 (br s, 1H), 5.93 (br s, 1H), 5.15 (s, 2H), 4.70 (s, 2H).

### Preparation of 2-bromo-N-(3-hydroxypropyl)-1,3-thiazole-4-carboxamide (i-37)

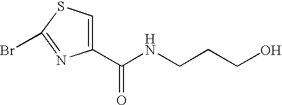

To a solution of 2-bromothiazole-4-carboxylic acid (500 mg, 2.4 mmol), and 3-aminopropan-1-ol (247 μl, 4.8 mmol) in CH₂Cl₂ (15 ml) was added N-(3-dimethylaminopropyl)-N′-ethylcarbodiimide hydrochloride (690 mg, 3.6 mmol), 1-hydroxybenzotriazole (486 mg, 3.6 mmol), and diisopropylethylamine (627 μl, 3.6 mmol). The resulting mixture was stirred at room temperature for 4 hours. The mixture was washed with water, 1N HCl, sat. NaHCO₃, sat. NaCl, dried over Na₂SO₄, filtered and evaporated to give the title compound. ¹HNMR (500 MHz, CDCl₃) δ: 8.08 (s, 1H), 7.55 (br s, 1H), 3.70 (q, J=5.7, 2H), 3.63 (q, J=6.4, 2H), 3.13 (t, J=6.2, 1H), 1.82 (m, 2H).

### Preparation of 2-bromo-N-(2-hydroxyethyl)-1,3-thiazole-4-carboxamide (i-38)

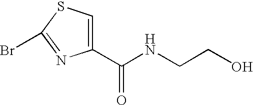

The title compound was prepared 2-bromothiazole-4-carboxylic acid and ethanolamine according to the procedure for intermediate (i-37). ¹HNMR (500 MHz, CDCl₃) δ: 8.08 (s, 1H), 7.65 (br s, 1H), 3.85 (t, J=5.0, 2H), 3.63 (q, J=5.7, 2H), 2.90 (br s, 1H).

### Preparation of Ethyl 2-aminooxazole-4-carboxylate (i-39)

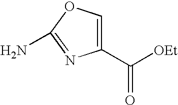

A mixture of ethyl bromopyruvate (59.7 g, 306 mmol) and urea (27.6, 460 mmol) in ethanol (220 ml) was heated at reflux for 24 hours. The mixture was cooled and evaporated. The residue was taken up in water and treated with 1N NaOH until the pH>9. The mixture was extracted with Et₂O (4×100 ml); the combined Et₂O layers were dried over Na₂SO₄, filtered and evaporated. The residue was purified by MPLC on silica gel eluting with a gradient rising from 100% hexanes to 90% EtOAc in hexanes. Product containing fractions were combined and evaporated and the residue triturated with EtOAc/hexanes filtered and dried to give the title compound. ¹HNMR (500 MHz, DMSO-d6) δ: 8.04 (s, 1H), 6.90 (br s, 2H), 4.18 (q, J=7.1, 2H), 1.22 (t, J=7.13H).

### Preparation of ethyl 2-chlorooxazole-4-carboxylate (i-40)

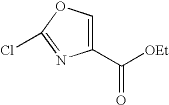

Intermediate 39 (3 g, 19.2 mmol) was added portionwise to a mixture of tert-butyl nitrite (93.4 ml, 28.8 mmol) and copper (II) chloride (3.87 g, 28.8 mmol) in acetonitrile (100 ml) warmed at 60° C. After complete addition the mixture was heated at 75° C. for 2 hours. The mixture was cooled and poured into 1N HCl (300 ml), and extracted with CH₂Cl₂ (3×120 ml). The combined CH₂Cl₂ extracts were dried over Na₂SO₄, filtered and evaporated to give of the title compound. ¹HNMR (500 MHz, CDCl₃) δ: 8.20 (s, 1H), 4.39 (q, J=7.3, 2H), 1.39 (t, J=7.3, 3H).

### Preparation 2-chlorooxazole-4-carboxamide (i-41)

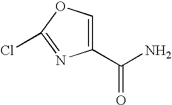

The title compound was prepared from intermediate 40 according to the procedure for intermediate (i-12). ¹HNMR (500 MHz, DMSO-d6) δ: 7.79 (s, 1H), 7.30 (br s, 1H), 7.10 (br s, 1H).

### Preparation of 2-chloropyrimidine-4-carboxamide (i-42)

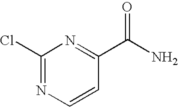

Lithium hydroxide (122 mg, 2.91 mmol) was dissolved in water (4 ml), and H₂O₂ (536 μl of a 30% solution in water, 4.89 mmol) added. This mixture was added to a solution of 2-chloro-4-cyanopyrimidine [prepared as described in WO 2006 072831 A1] (340 mg, 2.45 mmol) in THF (16 ml). The resulting mixture was stirred at room temperature for 2 hours. The mixture was partitioned between EtOAc and water; the organic layer was washed with more water, sat. NaCl, dried over Na₂SO₄, filtered and evaporated. The residue was triturated with Et₂O/hexanes, filtered and dried to give the title compound. ¹HNMR (500 MHz, CDCl₃) δ: 8.91 (d, J=4.8, 1H), 8.10 (d, J=4.8, 1H), 7.68 (br s, 1H), 5.99 (br s, 1H).

### Preparation of 5-bromo-2-cyanopyrimidine (i-43)

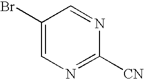

Sodium cyanide (270 mg, 5.43 mmol) was dissolved in water (3 ml) and DABCO (87 mg, 0.8 mmol) added, followed by DMSO (3 ml). To this mixture was added a solution of 5-bromo-2-chloropyrimidine (1 g, 5.17 mmol) in DMSO (3 ml), and the resulting mixture stirred at room temperature for 2 hours. The mixture was diluted with EtOAc (75 ml) and washed with water, 1N HCl, sat. NaHCO₃, filtered and evaporated to give of the title compound. ¹HNMR (500 MHz, CDCl₃) δ: 8.96 (s, 2H).

### Preparation of 5-bromopyrimidine-2-carboxamide (i-44)

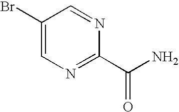

The title compound was prepared from intermediate 41 according to the procedure for intermediate (i-42). ¹HNMR (500 MHz, CDCl₃) δ: 8.97 (s, 2H), 7.72 (br s, 1H), 6.38 (br s, 1H).

### Preparation of 2-hydroxy-4-iodobenzaldehyde (i-45)

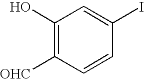

3-Iodophenol (10 g, 45 mmol) was dissolved in anhydrous acetonitrile (160 ml), cooled in an ice bath and magnesium chloride (12.8 g, 134 mmol) added portionwise over 10 mins. Triethylamine (25.3 ml, 363 mmol) was added to this mixture over 5 mins, followed by portionwise addition of paraformaldehyde (5.47 g, 636 mmol). After complete addition the mixture was heated at reflux for 18.5 hours. The mixture was cooled and quenched by the addition of sat. NH₄Cl (350 ml) and extracted with EtOAc (3×150 ml). The combined EtOAc layers were washed with sat. NaHCO₃ (2×150 mml), 1N HCl (2×150 ml), and sat. NaCl (2×100 ml), dried over Na₂SO₄, filtered and evaporated. The residue was purified by MPLC on silica gel eluting with a gradient rising from 100% hexanes to 20% EtOAc in hexanes. Product containing fractions were combined and evaporated and recrystallised from hot hexanes to give of the title compound. ¹HNMR (500 MHz, CDCl₃) δ: 11.02 (s, 1H), 9.87 (s, 1H), 7.46 (d, 1H), 7.42 (dd, 1H), 7.25 (d, 1H).

### Preparation of 2-benzyloxy-4-iodobenzaldehyde (i-46)

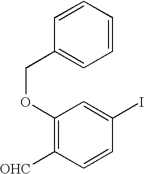

To a solution of 2-hydroxy-4-iodobenzaldehyde (i-45) (5 g, 20.2 mmol) in anhydrous acetonitrile (25 ml) was added 1,8-diazabicyclo[5.4.0]undec-7-ene (3.2 ml, 21.2 mmol), followed by benzyl bromide (2.53 ml, 21.2 mmol). The mixture was stirred at room temperature for 15 mins then warmed at 50° C. for 4 hours. The cooled reaction mixture was evaporated. The residue was partitioned between 1N HCl (150 ml) and Et₂O (150 ml), and extracted with Et₂O (3×150 ml). The combined Et₂O layers were washed with water (150 ml), sat. NaCl (150 ml), dried over MgSO₄, filtered and evaporated. The residue was recrystallized from EtOAc/hexanes to give of the title compound. ¹HNMR (500 MHz, CDCl₃) δ: 10.50 (s, 1H), 7.58 (d, 1H), 7.50-7.38 (m, 7H), 5.19 (s, 2H).

### Preparation of 4-({(1E)-[2-(benzyloxy)-4-iodophenyl]methylene}amino)phenol (i-47)

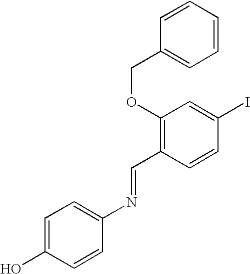

2-benzyloxy-4-iodobenzaldehyde (i-46) (1.1 g, 3.25 mmol) was suspended in propan-2-ol (10.5 ml) and warmed until complete dissolution. 4-hydroxyaniline (355 mg, 3.25 mmol) was added to the clear solution and the resulting mixture warmed at 50° C. for 4 hours. The cooled mixture was evaporated, and the residue triturated with a mixture of Et₂O and hexanes, filtered an air dried to give of the title compound. ¹HNMR (500 MHz, CD₃OD) δ: 8.84 (s, 1H), 7.75 (d, 1H), 7.52 (s, 1H), 7.48-7.30 (m, 6H), 7.10 (d, 2H), 6.80 (d, 2H), 5.20 (s, 2H).

### Preparation of 4-(2S,3R)-3-[(3S)-3-(acetyloxy)-3-(4-fluorophenyl)propyl]-2-[2-(benzyloxy)-4-iodophenyl]-4-oxoazetidin-1-yl}phenyl acetate (i-48)

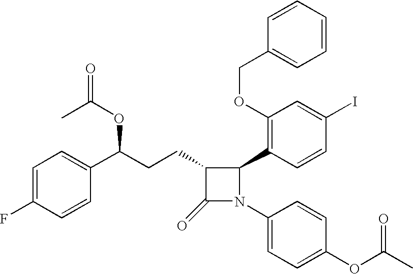

The title compound was prepared from 4-({(1E)-[2-(benzyloxy)-4-iodophenyl]methylene}amino)phenol (i-47), according to the procedures outlined in Example 1, steps B,C, and D. ¹HNMR (500 MHz, CDCl₃) δ: 7.48-7.35 (m, 6H), 7.31-7.20 (m, 4H), 7.11 (t, 2H), 7.01-6.90 (m, 4H), 5.58 (t, 1H), 5.10 (m, 2H), 4.97 (d, 1H), 3.04 (m, 1H), 2.29 (s, 3H), 2.01 (s, 3H), 2.00-1.88 (m, 2H), 1.88-1.68 (m, 2H).

### Preparation of (1S)-3-{(2S,3R)-2-(4-{4-(acetyloxy)-3-[(acetyloxy)methyl]-3-hydroxybut-1-yn-1-yl}phenyl)-4-oxo-1-[4-(4,4,5,5-tetramethyl-1,3,2-dioxaborolan-2-yl)phenyl]azetidin-3-yl}-1-(4-fluorophenyl)propyl acetate (i-49)

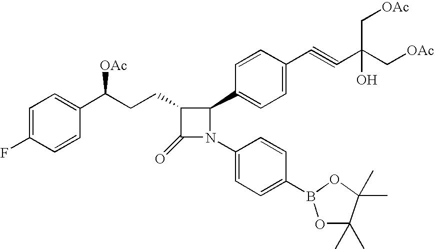

Nitrogen gas was bubbled through a solution of (1S)-3-[(2S,3R)-2-(4-{4-(acetyloxy)-3-[(acetyloxy)methyl]-3-hydroxybut-1-yn-1-yl}phenyl)-4-oxo-1-(4-{[(trifluoromethyl)sulfonyl]oxy}phenyl)azetidin-3-yl]-1-(4-fluorophenyl)propyl acetate (Intermediate from Example 1, step F), (1 g, 1.3 mmol), bis(pinacolato)diboron (366 mg, 1.4 mmol), and potassium acetate (382 mg, 3.9 mmol) in anhydrous 1,4-dioxane (15 ml) for 15 mins. [1,1′-Bis(diphenylphosphino)ferrocene]dichloropalladium(II) (95 mg, 0.13 mmol) was added to the mixture and the resulting mixture heated at 60° C. for 14 hours. The mixture was cooled and poured into water (80 ml), and extracted with EtOAc (3×50 ml). The combined EtOAc layers were washed with water (100 ml), sat. NaCl (50 ml), dried over Na₂SO₄, filtered and evaporated. The residue was purified by MPLC on silica gel eluting with a gradient rising from 100% hexanes to 40% EtOAc in hexanes. ¹HNMR (500 MHz, CDCl₃) δ: 7.47 (d, J=8.2, 2H), 7.32-7.27 (m, 6H), 7.16 (d, J=8.9, 2H), 7.04 (t, J=8.7, 2H), 5.72 (t, J=6.5, 1H), 4.63 (d, J=2.3, 1H), 4.39 (d, J=11.2, 2H), 4.32 (d, J=11.2, 2H), 3.14-3.12 (m, 2H), 2.16 (s, 6H), 2.11-2.03 (m, 5H), 1.94-1.86 (m, 2H), 1.26 (s, 12H).

### Preparation of 2-chloro-6-[(4-methyoxybenzyl)oxy]pyrazine (i-50)

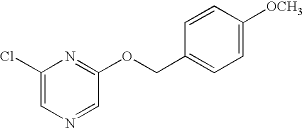

To a solution of 4-methoxybenzyl alcohol (186 mg, 2.68 mmol) in anhydrous DMF (5 mL) set under nitrogen atmosphere and cooled to 0° C. was added in portions solid NaH (60% dispersion in oil, 112 mg, 2.80 mmol) and the resulting solution stirred for 1 hour at 0° C. A pre-made solution on 2,6-dichloropyrazine (200 mg, 2.68 mmol) in DMF (1 mL) was introduced via syringe to the cooled solution and the resulting mixtures stirred overnight allowing to warm to room temperature. The reaction was quenched with saturated ammonium chloride solution (10 mL) and extracted with ethyl acetate (3×7 mL). The organics were combined, dried over sodium sulfate, filtered and concentrated. Preparative plate purification eluting with 10% ethyl acetate/90% hexane afforded the title compound. m/z (ES) 251 (MH)+ and 253 (M2+H)+.

### Preparation of 2-chloro-3-[(4-methyoxybenzyl)oxy]pyrazine (i-51)

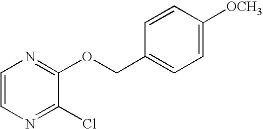

The title compound was prepared from 2,3-dichloropyrazine according to the procedure for intermediate (i-45). m/z (ES) 251 (MH)+ and 253 (M2+H)+.

### Preparation of Ethyl-3-iodo-1-trityl-1,2,4-triazole-5-carboxylate (i-52)

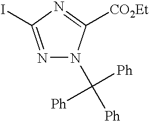

Ethyl-5-iodo-1H-1,2,4-triazole-3-carboxylate (Chinese Journal of Synthetic Chemistry, 12(2), 2004, page 191) in an anhydrous solvent such as DMF, may be treated with an organic base such as triethylamine and trityl chloride under an inert atmosphere such as nitrogen or argon. The mixture may be stirred at a temperature between 20° C. and 40° C. for a time between 1 hour and 24 hours. The reaction may be worked up by pouring into an excess of water and extracting with an organic solvent such as EtOAc, drying the organic extracts over a drying agent such as MgSO₄, or Na₂SO₄, filtering and evaporating under vacuum.

### Preparation of 3-iodo-1-trityl-1,2,4-triazole-5-carboxamide (i-53)

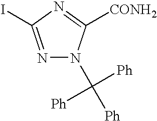

The title compound may be prepared by stirring ethyl-3-iodo-1-trityl-1,2,4-triazole-5-carboxylate with a solution of ammonia in an alcoholic solvent such as MeOH or EtOH in a sealed vessel at a temperature between 20° C. and 60° C. for a time between 1 hour and 36 hours. The title compound may be isolated by filtration of any precipitated product, or evaporation of the crude reaction mixture.

### Preparation of 5-cyano-3-iodo-1-trityl-1,2,4-triazole (i-54)

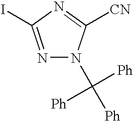

The title compound may be prepared by the slow addition of trifluoroacetic anhydride to a solution of 3-iodo-1-trityl-1,2,4-triazole-5-carboxamide and an organic base such as pyridine or triethylamine in an anhydrous solvent such as CH₂Cl₂ or 1,4-dioxane under an inert atmosphere such as nitrogen or argon at a temperature between 0° C. and 20° C. The mixture may be stirred at a temperature between 0° C. and 20° C. for a time between 1 and 12 hours. The reaction may be worked up by pouring into an excess of water and extracting with an organic solvent such as CH₂Cl₂ or EtOAc, drying the organic extracts over a drying agent such as MgSO₄, or Na₂SO₄, filtering and evaporating under vacuum.

### Preparation of 3-iodo-1-trityl-1,2,4-triazole-5-methanol (i-55)

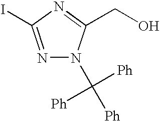

The title compound may be prepared by treating ethyl-3-iodo-1-trityl-1,2,4-triazole-5-carboxylate in an anhydrous solvent such as tetrahydrofuran or diethyl ether under an inert atmosphere such as nitrogen or argon with a reducing agent such as lithium aluminum hydride or lithium borohydride at a temperature between 0° C. and 20° C. The reaction may be stirred at a temperature between 0° C. and 40° C. for a time between 1 and 12 hours. The cooled reaction may worked up by the careful addition of 1N HCl, and extraction into an organic solvent such as CH₂Cl₂ or EtOAc, drying the organic extracts over a drying agent such as MgSO₄, or Na₂SO₄, filtering and evaporating under vacuum.

### Preparation of [(prop-3-yn-1-yloxy)methyl]benzene or benzyl prop-3-yn-1-yl ether (i-56)

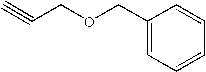

To a solution of 3-prop-1-ol (1.17 g, 11.88 mmol) in anhydrous DMF (100 mL) under nitrogen atmosphere was added TBAI (0.87 g, 2.38 mmol) followed by 60% NaH dispersion in oil (0.55 g, 14.26 mmol) in portions over 0.5 h. The reaction mixture was stirred for 0.5 hr at which time benzyl bromide (2.44 g, 14.26 mmol) was added by syringe. The reaction mixture was stirred for 16 h at room temperature at which time the reaction was quenched by the addition of sat. aq. NH₄Cl (100 mL). The reaction mixture was transferred to separatory funnel and extracted with ether (3×75 mL). The combined organic extracts were washed with water (50 mL), brine (75 mL), dried (Na₂SO₄), filtered and the solvent removed under vacuum. The residue was purified by MPLC (silica column) with stepwise gradient elution (0-60% EtOAc/hexanes as eluent) to afford the title compound (i-56).

Intermediates related to those described above of varying substitution and alkyl chain length may be prepared from the appropriate starting materials using the procedures described above.

### Example 1

### (3R,4S)-4-{4-[3,4-dihydroxy-3-(hydroxymethyl)butyl]phenyl}-3-[(3S)-3-(4-fluorophenyl)-3-hydroxypropyl]-1-{4-[3-(1H-1,2,4-triazol-1-yl)propyl]phenyl}azetidin-2-one

### Step A: Preparation of 4-{[(1E)-(4-iodophenyl)methylene]amino}phenol

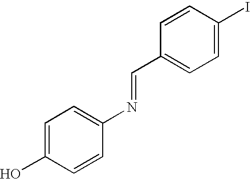

To a round bottom flask under nitrogen atmosphere was added iodobenzaldehyde (400 g, 1.724 mol) which was then dissolved in 2-propanol (950 ml). 4-hydroxyaniline was added and the resulting mixture heated to 70° C. After heating at that temperature for 3 h, a tan precipitate formed in the dark brown solvent mixture. The reaction mixture was cooled, filtered, washed with 2-propanol then ether. The organics were evaporated in vacuo and the residue was dried under high vacuum overnight to afford the title compound which was used without further purification. ¹HNMR (500 MHz, DMSO-D6) δ: 9.55 (s, 1H), 8.59 (s, 1H), 7.85 (d, 2H), 7.63 (d, 2H), 7.2 (d, 2H), 6.80 (d, 2H).

### Step B: Preparation of (4S)-3-{(2R)-5-(4-fluorophenyl)-2-[(S)-(4-iodophenyl)({4-[trimethylsilyl)oxy]phenyl}amino)methyl]-5-(trimethylsilyl)oxy]pentanoyl}-4-phenyl-1,3-oxazolidin-2-one

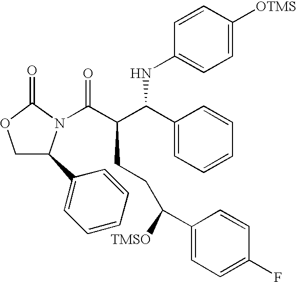

To a suspension of (4S)-3-[(5S)-5-(4-fluorophenyl)-5-hydroxypentanoyl]-4-phenyl-1,3-oxazolidin-2-one (251.6 g, 0.704 mol) (prepared according to the procedures of Fu, X.; McCallister, T. L.; Thiruvengadam, T. K.; Tann, C. H.; and Su, D. Tetrahedron Lett. (2003) 44, 801-804) and 4-{[(1E)-(4-iodophenyl)methylene]amino}phenol (455 g, 1.41 mol; intermediate step A) in CH₂Cl₂ (3.1 L) under nitrogen atmosphere at −5° C. was added N,N-diisopropylethylamine (640 mL, 3.66 mol) keeping the temperature below 0° C. To the resulting yellow suspension was added chlorotrimethylsilane (297 mL, 2.323 mol) keeping the temperature below 0° C. The resulting dark red solution was stirred at −5° C. for 1 h at which time the reaction mixture was cooled to −30° C. To this cooled solution was added TiCl₄ (90 mL, 0.774 mol) keeping the temperature below −25° C. The resulting dark purple solution was stirred at −30° C. for 2.5 hrs at which time acetic acid (210 mL) was added keeping the temperature below −25° C. After the completion of the addition, the reaction mixture was poured into a pre-cooled 0° C. solution of Rochelle's salt (245 g, potassium sodium tartrate) in water (3.5 L) cooled in an ice/salt bath. The resulting mixture was stirred at 0° C. for 1 hr at which time a solution of sodium hydrogensulfite (250 g) in water (1.25 L) was added. The resulting solution was stirred at ambient temperature overnight. Filter aid was added to the mixture; the reaction mixture was then filtered through a pad of filter aid. The solids were washed with CH₂Cl₂ and the filtrates transferred to a separatory funnel. The layers were separated and the aqueous layer extracted with CH₂Cl₂ (3 L). The combined organic layers were washed with water, dried over MgSO₄, filtered and the solvent removed under vacuum until ˜2 L of a dark red solution remained. This mixture was placed in a round bottom flask under nitrogen atmosphere and N,O-bis(trimethylsilyl)acetamide (216 mL, 0.866 mol) was added. After completion of the addition, the mixture was heated to 45° C., then kept at that temperature for 0.5 hr. The reaction mixture was cooled, concentrated under vacuum until a light orange solid formed. A small amount of methyl-t-butylether was added followed by heptane (2 L). The resulting suspension was stirred for ten minutes, filtered and the resulting solid washed with heptane. The resulting solid was dried under vacuum at 60° C. overnight to afford the title compound, which was used without further purification.

### Step C: Preparation of (3R,4S)-3-[(3S)-3-(4-fluorophenyl)-3-hydroxypropyl]-1-(4-hydroxyphenyl)-4-(4-iodophenyl)azetidin-2-one

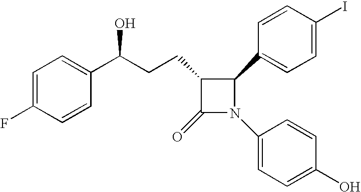

To a suspension of (4S)-3-{(2R)-5-(4-fluorophenyl)-2-[(5)-(4-iodophenyl)({4-[(trimethylsilyl)oxy]phenyl}amino)methyl]-5-[(trimethylsily)oxy]pentanoyl}-4-phenyl-1,3-oxazolidin-2-one (22.13 g, 26.83 mmol; intermediate of step B) in methyl-t-butylether (180 mL) was added N,O-bis(trimethylsilyl)acetamide (12 mL, 45.61 mmol) followed by tetra-n-butylammonium fluoride (0.45 g, 1.34 mmol). The reaction mixture was stirred at ambient temperature for 2.5 h at which time acetic acid (1.10 mL) was added then the mixture was stirred for ten minutes. The solvent was removed under vacuum to give a yellow oil. The oil was dissolved in 2-propanol (110 mL) and then a solution of 2N aq. H₂SO₄ (11 mL) was added. The resulting mixture was stirred at RT for ˜16 h then poured into a separatory funnel containing water and ethyl acetate. The layers were separated and the aqueous layer extracted with EtOAc. The combined organic layers were dried over MgSO₄, filtered and the solvent removed under vacuum. The residue (19.7 g dissolved in a minimal amount of CH₂Cl₂) was purified by MPLC on silica gel eluting with gradient from 20% EtOAc/heptane to 60% EtOAc/heptane to afford the title compound, which contained a minor amount of oxazolidinone impurity.

### Step D: Preparation of 4-[(2S,3R)-3-[(3S)-3-(acetyloxy)-3-(4-fluorophenyl)propyl]-2-(4-iodophenyl)-4-oxoazetidin-1-yl]phenyl acetate

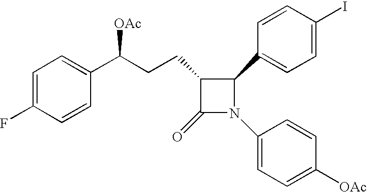

To a solution of the intermediate of step C (16.6 g, ˜26.8 mmol) in CH₂Cl₂ (145 mL) was added anhydrous pyridine (2.6 mL, 32.2 mmol), acetic anhydride (3.1 mL, 32.2 mmol) and DMAP (0.2 g, ˜1.3 mmol). The reaction mixture was stirred at RT for 1 hr at which time was added pyridine (1.8 mL, 0.8 equiv.), acetic anhydride (2.1 mL, ˜0.8 equiv.). The reaction mixture was stirred at RT for another 1 hr at which time was added pyridine (0.5 mL, ˜0.23 equiv.), acetic anhydride (0.5 mL, ˜0.20 equiv.). The reaction mixture was stirred at ambient temperature for 16 hr then poured into a separatory funnel which contained a solution of 1N aq. HCl (200 mL). The layers were separated and the organic layer was washed with sat. aq. NaHCO₃, dried over MgSO₄, filtered and the solvent removed under vacuum. The residue dissolved in a minimal amount of CH₂Cl₂ was purified by MPLC on silica gel eluting with gradient from 20% EtOAc/heptane to 50% EtOAc/heptane to afford the title compound. ¹HNMR (500 MHz, CDCl₃) δ: 7.74 (d, J=8.5, 2H), 7.29 (m, 2H), 7.26 (d, J=8.9, 2H), 7.10 (d, J=8.2, 2H), 7.05 (t, J=8.5, 2H), 6.99 (d, J=8.7, 2H), 5.72 (t, J=6.9, 1H), 4.57 (d, J=2.1, 1H), 3.08 (m, 1H), 2.29 (s, 3H), 2.08 (s, 3H), 2.08-2.01 (m, 2H), 1.92-1.85 (m, 2H).

### Step E: Preparation of (1S)-1-(4-fluorophenyl)-3-[(2S,3R)-2-(4-iodophenyl)-4-oxo-1-(4-{[(trifluoromethyl)sulfonyl]oxy}phenyl)azetidin-3-yl]propyl acetate

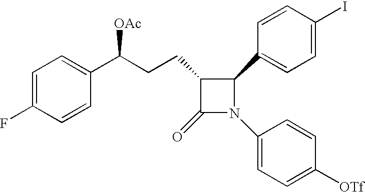

To a solution of 4-[(2S,3R)-3-[(3S)-3-(acetyloxy)-3-(4-fluorophenyl)propyl]-2-(4-iodophenyl)-4-oxoazetidin-1-yl]phenyl acetate (18 g; 30 mmol; intermediate step D) in MeOH (100 mL) was added guanidine (2.9 g, 30 mmol) followed by TEA (4.2 mL, 30 mmol). The resulting mixture was stirred at RT for 3 hr at which time the solvent was removed under vacuum. The residue was dissolved in EtOAc (400 mL) and 1N aq. HCl (200 mL). The layers were separated and the organic layer was washed with brine (200 mL), dried over MgSO₄, filtered and the solvent removed under vacuum.

The material obtained above was dissolved in CH₂Cl₂ (100 ml) and then pyridine (2.67 mL, 33 mmol) and trifluoromethanesulfonic anhydride (5.55 mL; 33 mmol) were added simultaneously by separate syringes over a 20 minute period. The reaction mixture stirred for 1 h. The reaction mixture was washed with 1N aq. hydrochloric acid (100 mL) and then brine (100 ml), dried over anhydrous MgSO₄ powder, filtered, and the solvent evaporated under reduced pressure to leave a yellow oil. The oil was purified by MPLC on silica gel eluting with gradient from 0% EtOAc/hexane to 70% EtOAc/hexane to afford the title compound. ¹HNMR (500 MHz, CDCl₃) δ: 7.76 (d, J=8.2, 2H), 7.32-7.28 (m, 4H), 7.18 (d, J=8.9, 2H), 7.10 (d, J=8.2, 2H), 7.05 (t, J=8.7, 2H), 5.73 (t, J=6.7, 1H), 4.59 (d, J=2.5), 3.12 (m, 1H), 2.08 (s, 3H), 2.08-2.02 (m, 2H), 1.93-1.86 (m, 2H).

### Step F: Preparation of (1S)-3-[(2S,3R)-2-(4-{4-(acetyloxy)-3-[(acetyloxy)methyl]-3-hydroxybut-1-yn-1-yl}phenyl)-4-oxo-1-(4-{[(trifluoromethyl)sulfonyl]oxy}-phenyl)azetidin-3-yl]-1-(4-fluorophenyl)propyl acetate

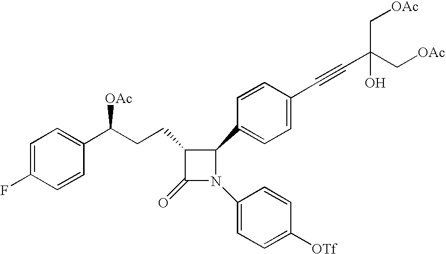

Nitrogen gas was bubbled through a solution of (1S)-1-(4-fluorophenyl)-3-[(2S,3R)-2-(4-iodophenyl)-4-oxo-1-(4-{[(trifluoromethyl)sulfonyl]oxy}phenyl)azetidin-3-yl]propyl acetate (10.25 g, 14.83 mmol; intermediate step E), 2-ethynylpropane-1,2,3-triol 1,3-diacetate (3.86 g, 19.28 mmol; intermediate i-2) and triethylamine (14.47 mL, 104 mmol) in anhydrous DMF (100 mL) for 15 minutes. Pd(PPh₃)Cl₂ (1.04 g, 1.48 mmol) and CuI (0.057 g, 2.97 mmol) were added and the reaction mixture was stirred under nitrogen atmosphere for 1.5 h. The reaction mixture was poured into water (500 mL) and extracted with EtOAc (3×150 mL). The combined organic layers were washed with water (2×500 mL), brine (200 mL) dried over Na₂SO₄, filtered and the solvent removed under vacuum. The residue was purified by MPLC on silica gel with gradient from 0% EtOAc/hexanes to 50% EtOAc/hexanes then 50% EtOAc/hexanes to afford the title compound. ¹HNMR (500 MHz, CDCl₃) δ: 7.47 (d, J=8.2, 2H), 7.31-7.27 (m, 6H), 7.16 (d, J=9.1, 2H), 7.04 (t, J=8.5, 2H), 5.72 (t, J=6.6, 1H), 4.63 (d, J=2.1, 1H), 4.39 (d, J=11.4, 2H), 4.32 (d, J=11.4, 2H), 3.12 (m, 2H), 2.16 (s, 6H), 2.08 (s, 3H), 2.08-2.02 (m, 2H), 1.93-1.86 (m, 2H).

### Step G: Preparation of (1S)-3-((2S,3R)-2-(4-{4-(acetyloxy)-3-[(acetyloxy)methyl]-3-hydroxybut-1-yn-1-yl}phenyl)-4-oxo-1-{4-[3-(1H-1,2,4-triazol-1-yl)prop-1-yn-1-yl]phenyl}azetidin-3-yl)-1-(4-fluorophenyl)propyl acetate

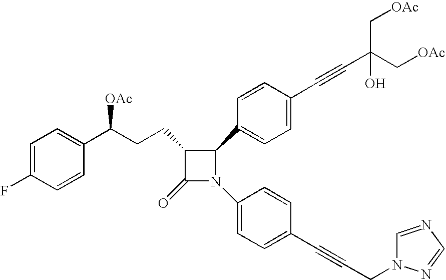

Nitrogen gas was bubbled through a solution of (1S)-3-[(2S,3R)-2-(4-{4-(acetyloxy)-3-[(acetyloxy)methyl]-3-hydroxybut-1-yn-1-yl}phenyl)-4-oxo-1-(4-{[(trifluoromethyl)sulfonyl]oxy}phenyl)azetidin-3-yl]-1-(4-fluorophenyl)propyl acetate (300 mg, 0.43 mmol; intermediate step F), 1-prop-2-yn-1-yl-1H-1,2,4-triazole (i-7) (229 mg, 2.1 mmol), triethylamine (0.29 mL, 2.1 mmol) and tetra-n-butylammonium iodide (159 mg, 0.43 mmol) in anhydrous DMF (5 mL) for 15 minutes. Pd(PPh₃)₄ (50 mg, 0.043 mmol) and CuI (4 mg, 0.022 mmol) were added and the reaction mixture was heated at 70° C. under nitrogen atmosphere for 3 days. The reaction mixture was cooled to RT, poured into water (50 mL) and extracted with EtOAc (3×20 mL). The combined organic layers were washed with water (2×50 mL), brine (25 mL) dried over Na₂SO₄, filtered and the solvent removed under vacuum. The residue was purified by MPLC on silica gel eluting with gradient from 0% EtOAc/hexanes to 90% EtOAc/hexanes then 90% EtOAc/hexanes to afford the title compound. ¹HNMR (500 MHz, CDCl₃) δ: 8.48 (s, 1H), 8.06 (s, 1H), 7.47 (d, J=8.2, 2H, 7.36 (d, J=8.7, 2H), 7.30 (m, 4H), 7.20 (d, J=8.7, 2H), 7.06 (t, J=8.7, 2H), 5.72 (t, J=6.9, 1H), 5.22 (s, 1H), 4.65 (d, J=2.3, 1H), 4.40 (d, J=11.2, 2H), 4.33 (d, J=11.2, 2H), 3.10 (m, 1H), 2.17 (s, 6H), 2.09 (s, 3H), 2.09-2.03 (m, 2H), 1.94-1.87 (m, 2H).

### Step H: Preparation of (1S)-3-((2S,3R)-2-(4-{4-(acetyloxy)-3-[(acetyloxy)methyl]-3-hydroxybutyl}phenyl)-4-oxo-1-{-[3-(1H-1,2,4-triazol-1-yl)propyl]phenyl}azetidin-3-yl)-1-(4-fluorophenyl)propyl acetate

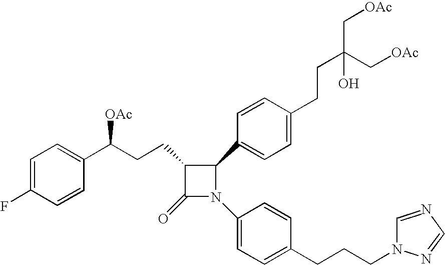

To a solution of (1S)-3-((2S,3R)-2-(4-{4-(acetyloxy)-3-[(acetyloxy)methyl]-3-hydroxybut-1-yn-1-yl}phenyl)-4-oxo-1-{4-[3-(1H-1,2,4-triazol-1-yl)prop-1-yn-1-yl]phenyl}azetidin-3-yl)-1-(4-fluorophenyl)propyl acetate (intermediate Step G; 65 mg, 0.09 mmol) in EtOAc/EtOH (4 mL; 10/1) flushed with nitrogen gas was added 10% Pd—C (15 mg). The resulting mixture was stirred under hydrogen atmosphere at room pressure for 16 hrs. The catalyst was removed by filtration through filter aid and the solvent removed under vacuum. The residue was purified by preparative plate eluting with MeOH/CH₂Cl₂ (90/10) to provide the title compound. ¹HNMR (500 MHz, CDCl₃) δ: 8.08 (s, 1H), 7.99 (s, 1H), 7.31-7.27 (m, 4H), 7.24-7.21 (m, 4H), 7.06-7.03 (m, 4H), 5.73 (t, J=6.9, 1H), 4.60 (d, J=2.2, 1H), 4.18-4.11 (m, 6H), 3.27 (m, 3H), 3.18 (m, 1H), 2.77 (m, 2H), 2.57 (t, J=7.6, 2H), 2.20 (t, J=7.6, 2H), 2.13 (s, 6H), 2.08 (s, 3H), 2.07-2.04 (m, 2H), 1.91-1.86 (m, 4H), 1.48 (m, (3H).

### Step I: Preparation of (3R,4S)-4-{4-[3,4-dihydroxy-3-(hydroxymethyl)butyl]phenyl}-3-[(3S)-3-(4-fluorophenyl)-3-hydroxypropyl]-1-{4-[3-(1H-1,2,4-triazol-1-yl)propyl]phenyl}azetidin-2-one

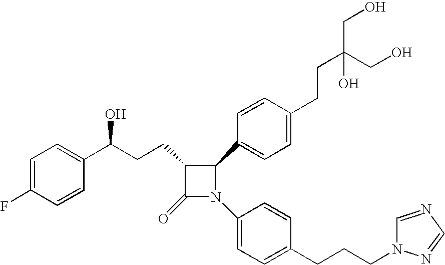

To a solution of (1S)-3-((2S,3R)-2-(4-{4-(acetyloxy)-3-[(acetyloxy)methyl]-3-hydroxybutyl}phenyl)-4-oxo-1-{4-[3-(1H-1,2,4-triazol-1-yl)propyl]phenyl}azetidin-3-yl)-1-(4-fluorophenyl)propyl acetate (35 mg, 0.05 mmol; intermediate step H) in EtOH (3 mL) was added potassium trimethylsilanoate (2 mg, 0.014 mmol). The resulting mixture was stirred at RT for 16 hrs. The reaction mixture was purified by prep HPLC (C-18 Sunfire column) eluting with gradient CH₃CN/0.1% aq. TFA (5 to 90%). The product fractions were collected and freeze dried from CH₃CN/water to afford the title compound. m/z (ES) 603 (MH)⁺, ¹HNMR (500 MHz, DMSO-d6) δ: 8.48 (s, 1H), 7.94 (s, 1H), 7.29 (m, 4H), 7.18 (d, J=8.0, 2H), 7.11 (m, 6H), 5.26 (d, J=4.5, 1H), 4.84 (d, J=2.2, 1H), 4.49 (q, J=6.4, 1H), 4.39 (t, J=5.7, 2H), 4.12 (t, J=7.1, 2H), 4.05 (s, 1H), 3.29 (m, 4H), 3.06 (m, 1H), 2.59 (m, 2H), 2.43 (t, J=7.4, 2H), 2.01 (m, 2H), 1.83 (m, 1H), 1.72 (m, 3H), 1.58 (m, 2H)

### Example 2

### (3R,4S)-4-{4-[3,4-dihydroxy-3-(hydroxymethyl)butyl]phenyl}-3-[(3S)-3-(4-fluorophenyl)-3-hydroxypropyl]-1-{4-[2-(1H-1,2,4-triazol-5-yl)ethyl]phenyl}azetidin-2-one

### Step A: Preparation of (1S)-3-((2S,3R)-2-(4-{4-(acetyloxy)-3-[(acetyloxy)methyl]-3-hydroxybut-1-yn-1-yl}phenyl)-4-oxo-1-{4-[(trimethylsilyl)ethynyl]phenyl}azetidin-3-yl)-1-(4-fluorophenyl)propyl acetate

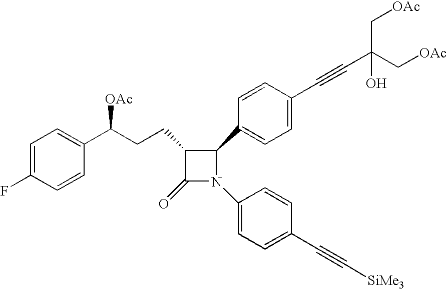

Nitrogen gas was bubbled through a solution of (1S)-3-[(2S,3R)-2-(4-{4-(acetyloxy)-3-[(acetyloxy)methyl]-3-hydroxybut-1-yn-1-yl}phenyl)-4-oxo-1-(4-{[(trifluoromethyl)sulfonyl]oxy}phenyl)azetidin-3-yl]-1-(4-fluorophenyl)propyl acetate (9.77 g, 12.8 mmol; intermediate step F, Example 1), trimethylsilylacetylene (4.52 mL, 32 mmol), tetra-n-butylammonium iodide (4.72 g, 12.8 mmol) and triethylamine (8.92 mL, 64 mmol) in anhydrous DMF (100 mL) for 15 minutes. Pd(PPh₃)₄ (1.48 g, 1.28 mmol) and CuI (0.49 g, 2.56 mmol) were added and the reaction mixture was heated at 50° C. under nitrogen atmosphere for 16 hr. The reaction mixture was cooled to RT, poured into water (500 mL) and extracted with EtOAc (3×200 mL). The combined organic layers were washed with water (2×500 mL), brine (200 mL) dried over Na₂SO₄, filtered and the solvent removed under vacuum. The residue was purified by MPLC on silica gel eluting with gradient from 0% EtOAc/hexanes to 40% EtOAc/hexanes then 40% EtOAc/hexanes to afford the title compound. ¹HNMR (500 MHz, CDCl₃) δ: 7.45 (d, J=8.0, 2H), 7.34 (d, J=8.7, 2H), 7.29 (m, 4H), 7.15 (d, J=8.7, 2H), 7.04 (t, J=8.7, 2H), 5.72 (t, J=6.6, 1H), 4.63 (d, J=2.1, 1H), 4.39 (d, J=11.4, 2H), 4.32 (d, J=11.4), 3.09-3.05 (m, 2H), 2.16 (s, 6H), 2.08 (s, 3H), 2.07-2.01 (m, 2H), 1.93-1.86 (m, 2H), 0.24 (s, 9H).

### Step B: Preparation of (1S)-3-[(2S,3R)-2-(4-{4-(acetyloxy)-3-[(acetyloxy)methyl]-3-hydroxybut-1-yn-1-yl}phenyl)-1-(4-ethynylphenyl)-4-oxoazetidin-3-yl]-1-(4-fluorophenyl)propyl acetate

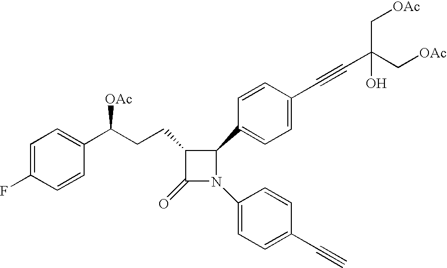

To a solution of (1S)-3-((2S,3R)-2-(4-{4-(acetyloxy)-3-[(acetyloxy)methyl]-3-hydroxybut-1-yn-1-yl}phenyl)-4-oxo-1-{4-[(trimethylsilyl)ethynyl]phenyl}azetidin-3-yl)-1-(4-fluorophenyl)propyl acetate (5.7 g, 8 mmol; intermediate step A) in anhydrous THF (60 mL) cooled to 0° C. in an ice bath was added slowly a 1.0M solution of tetra-n-butylammonium fluoride (8 mL, 8 mmol). The reaction mixture was stirred with continued cooling for 0.5 hr. The reaction mixture was diluted with water (150 mL) and extracted with CH₂Cl₂ (150 mL). The organic layer was dried over Na₂SO₄, filtered and the solvent removed under vacuum. The residue was purified by MPLC on silica gel eluting with 0% EtOAc/hexanes then gradient from 0% EtOAc/hexanes to 45% EtOAc/hexanes then 45% EtOAc/hexanes to afford the title compound. ¹HNMR (500 MHz, CDCl₃) δ: 7.45 (d, J=8.2, 2H), 7.37 (d, J=8.7, 2H), 7.29 (m, 4H), 7.18 (d, J=8.7, 2H), 7.04 (t, J=8.7, 2H), 5.72 (t, J=6.6, 1H), 4.63 (d, J=2.3, 1H), 4.38 (d, J=11.2, 2H), 4.31 (d, J=11.2, 2H), 3.11 (s, 1H), 3.08 (m, 1H), 3.04 (s, 1H), 2.16 (s, 6H), 2.08 (s, 3H), 2.07-2.02 (m, 2H), 1.93-1.86 (m, 2H).

### Step C: Preparation of (1S)-3-((2S,3R)-2-(4-{4-(acetyloxy)-3-[(acetyloxy)methyl]-3-hydroxybut-1-yn-1-yl}phenyl)-4-oxo-1-{4-[(1-trityl-1H-1,2,4-triazol-3-yl)ethynyl]phenyl}azetidin-3-yl)-1-(4-fluorophenyl)propyl acetate

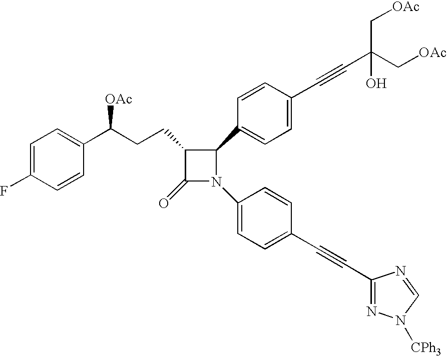

Nitrogen gas was bubbled through a solution of (1S)-3-[(2S,3R)-2-(4-{4-(acetyloxy)-3-[(acetyloxy)methyl]-3-hydroxybut-1-yn-1-yl}phenyl)-1-(4-ethynylphenyl)-4-oxoazetidin-3-yl]-1-(4-fluorophenyl)propyl acetate (4.0 g, 6.3 mmol; intermediate step B), 3-iodo-1-trityl-1H-1,2,4-triazole (i-8) (5.47 g, 12.5 mmol), triethylamine (4.4 mL, 31.3 mmol), and tetra-n-butylammonium iodide (2.31 g, 6.3 mmol) in anhydrous DMF (5 mL) and was heated at 50° C. for 20 minutes. Pd(PPh₃)₄ (0.72 g, 0.63 mmol) and CuI (0.2 g, 1.25 mmol) were added and the reaction mixture was heated at 50° C. under nitrogen atmosphere for ˜18 hr. The reaction mixture was cooled to RT, poured into water (700 mL) and extracted with EtOAc (3×200 mL). The combined organic layers were washed with water (2×500 mL), brine (200 mL) dried over Na₂SO₄, filtered and the solvent removed under vacuum. The residue was purified by column chromatography eluting with 0% EtOAc/hexanes then gradient from 0% EtOAc/hexanes to 50% EtOAc/hexanes then 50% EtOAc/hexanes to 60% EtOAc/hexanes then 60% EtOAc/hexanes to afford the title compound. ¹HNMR (500 MHz, CDCl₃) δ: 8.03 (s, 1H), 7.45 (d, J=8.0, 2H), 7.44 (d, J=8.7, 2H), 7.36 (m, 9H), 7.29 (m, 4H), 7.19 (d, J=8.7, 2H), 7.15 (m, 6H), 7.04 (t, J=8.4, 2H), 5.71 (t, J=6.6, 1H), 4.63 (d, J=2.3, 1H), 4.39 (d, J=11.5, 2H), 4.32 (d, J=11.5, 2H), 3.36 (s, 1H), 3.08 (m, 1H), 2.15 (s, 6H), 2.07 (s, 3H), 2.07-2.02 (m, 2H), 1.93-1.85 (m, 2H).

### Step D: Preparation of (1S)-3-{(2S,3R)-2-(4-{4-(acetyloxy)-3-[(acetyloxy)methyl]-3-hydroxybut-1-yn-1-yl}phenyl)-4-oxo-1-[4-(1H-1,2,4-triazol-5-ylethynyl)phenyl]azetidin-3-yl}-1-(4-fluorophenyl)propyl acetate

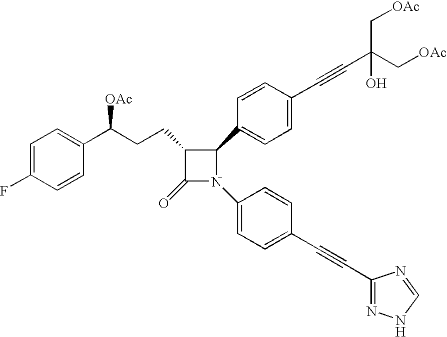

To a solution of (1S)-3-((2S,3R)-2-(4-{4-(acetyloxy)-3-[(acetyloxy)methyl]-3-hydroxybut-1-yn-1-yl}phenyl)-4-oxo-1-{4-[(1-trityl-1H-1,2,4-triazol-3-yl)ethynyl]phenyl}azetidin-3-yl)-1-(4-fluorophenyl)propyl acetate (600 mg, 0.63 mmol; intermediate step C), in acetone (10 mL) was added a solution of 1N aq. HCl (3 mL). The reaction mixture was stirred at RT for 16 hr. The reaction mixture was poured into sat. aq. NaHCO₃ (60 mL) and extracted with CH₂Cl₂ (3×30 mL). The combined organic layers were dried over Na₂SO₄, filtered and the solvent removed under vacuum. The residue was purified by column chromatography eluting with gradient from 0% EtOAc/hexanes to 100% EtOAc/hexanes then 100% EtOAc/hexanes to afford the title compound. ¹HNMR (500 MHz, CDCl₃) δ: 8.28 (s, 1H), 7.43 (d, J=8.0, 2H), 7.37 (d, J=8.5, 2H), 7.29 (m, 4H), 7.18 (d, J=8.7, 2H), 7.03 (t, J=8.7, 2H), 5.72 (t, J=6.6, 1H), 4.65 (d, J=2.1, 1H), 4.39 (d, J=11.5, 2H), 4.33 (d, J=11.5, 2H), 3.10 (m, 1H), 2.15 (s, 6H), 2.08 (s, 3H), 2.07-2.00 (m, 2H), 1.93-1.86 (m, 2H).

### Step E: Preparation of (1S)-3-((2S,3R)-2-(4-{4-(acetyloxy)-3-[(acetyloxy)methyl]-3-hydroxybutyl}phenyl)-4-oxo-1-{4-[2-(1H-1,2,4-triazol-5-yl)ethyl]phenyl}azetidin-3-yl)-1-(4-fluorophenyl)propyl acetate

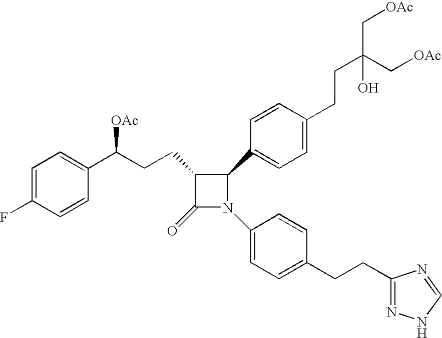

To a solution of (1S)-3-{(2S,3R)-2-(4-{4-(acetyloxy)-3-[(acetyloxy)methyl]-3-hydroxybut-1-yn-1-yl}phenyl)-4-oxo-1-[4-(1H-1,2,4-triazol-5-ylethynyl)phenyl]azetidin-3-yl}-1-(4-fluorophenyl)propyl acetate (176 mg, 0.25 mmol; intermediate Step D) in EtOAc/EtOH (12 mL; 5/1) flushed with nitrogen gas was added 10% Pd—C (50 mg). The resulting mixture was stirred under hydrogen atmosphere at room pressure for 16 hrs. The catalyst was removed by filtration through filter aid and the solvent removed under vacuum. The residue was purified by preparative plate eluting with MeOH/CH₂Cl₂ (85/15) to provide the title compound. ¹HNMR (500 MHz, CDCl₃) δ: 8.01 (s, 1H), 7.29-7.23 (m, 4H), 7.20 (d, J=8.0, 2H), 7.15 (d, J=8.5, 2H), 7.04-7.00 (m, 4H), 5.71 (t, J=6.6, 1H), 4.58 (d, J=2.3, 1H), 4.13 (m, 4H), 3.09-3.03 (m, 3H), 3.02-2.87 (m, 2H), 2.75 (m, 2H), 2.11 (s, 6H), 2.06 (s, 3H), 2.06-2.00 (m, 2H), 1.90-1.84 (m, 4H).

### Step F: Preparation of (3R,4S)-4-{4-[3,4-dihydroxy-3-(hydroxymethyl)butyl]phenyl}-3-[(3S)-3-(4-fluorophenyl)-3-hydroxypropyl]-1-{4-[2-(1H-1,2,4-triazol-5-yl)ethyl]phenyl}azetidin-2-one

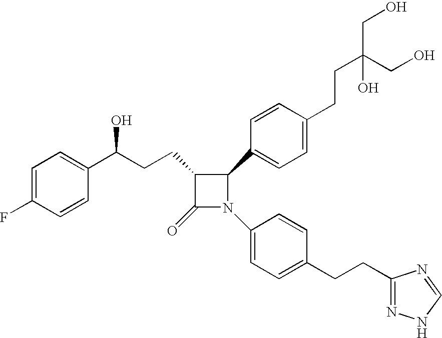

To a solution of (1S)-3-((2S,3R)-2-(4-{4-(acetyloxy)-3-[(acetyloxy)methyl]-3-hydroxybutyl}phenyl)-4-oxo-1-{4-[2-(1H-1,2,4-triazol-5-yl)ethyl]phenyl}azetidin-3-yl)-1-(4-fluorophenyl)propyl acetate (10.33 g, 14.47 mmol; intermediate step E) in anhydrous EtOH (175 mL) was added potassium trimethylsilanoate (2.43 g, 18.8 mmol). The resulting mixture was stirred at RT for 2 hours. The reaction mixture was adjusted to pH=5-6 by addition of conc. HCl (˜1.25 mL), evaporated under vacuum to a volume of ˜70 ml. The reaction mixture was filtered to remove a precipitate and then the filtrate was purified by prep HPLC (Column: C-18 Sunfire OBD 5 μm 30×100 mm) 750 μL injections eluting with a gradient CH₃CN/0.1% aq. TFA (20 to 40%). The combined product fractions were collected, and the resulting solution neutralized by addition of sat. aq. NaHCO₃, the majority of the organic solvent was removed under vacuum, and a white crystalline material precipitated. The solid was filtered and dried under vacuum to afford the title compound. Mpt 104° C. m/z (ES) 589 (MH)⁺; ¹H NMR (500 MHz, DMSO-d6+D₂O) δ: 8.01 (s, 1H), 7.29-7.26 (m, 4H), 7.18 (d, J=8.0, 2H), 7.11-7.06 (m, 6H), 4.82 (d, J=1.9, 1H), 4.47 (t, J=6.2, 1H), 3.28 (m, 4H), 3.04 (m, 1H), 2.87 (s, 4H), 2.58 (m, 2H), 1.86-1.78 (m, 1H), 1.75-1.66 (m, 3H), 1.56 (m, 2H).

### Example 3

### (3R,4S)-4-{4-[3,4-dihydroxy-3-(hydroxymethyl)butyl]phenyl}-3-[(3S)-3-(4-fluorophenyl)-3-hydroxypropyl]-1-{4-[3-(1,3-thiazol-2-ylamino)propyl]-phenyl}azetidin-2-one

### Step A: Preparation of (1S)-3-[(2S,3R)-2-(4-{4-(acetyloxy)-3-[(acetyloxy)methyl]-3-hydroxybut-1-yn-1-yl}phenyl)-4-oxo-1-(4-{[(trifluoromethyl)sulfonyl]oxy}-phenyl)azetidin-3-yl]-1-(4-fluorophenyl)propyl acetate

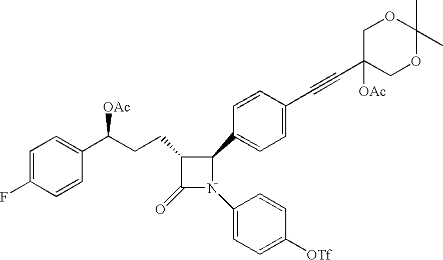

Nitrogen gas was bubbled through a solution of (1S)-1-(4-fluorophenyl)-3-[(2S,3R)-2-(4-iodophenyl)-4-oxo-1-(4-{[(trifluoromethyl)sulfonyl]oxy}phenyl)azetidin-3-yl]propyl acetate (5 g, 8.31 mmol; intermediate Example 1, Step E), 5-ethynyl-2,2-dimethyl-1,3-dioxan-5-yl acetate (2.48 g, 12.52 mmol; intermediate i-1) and triethylamine (8.10 mL, 58.17 mmol) in anhydrous DMF (75 mL) for 15 minutes. Pd(PPh₃)Cl₂ (633 mg, 0.90 mmol) and CuI (0.316 g, 1.66 mmol) were added and the reaction mixture was stirred under nitrogen atmosphere for 1.5 h. The reaction mixture was poured into water (250 mL) and extracted with EtOAc (3×100 mL). The combined organic layers were washed with water (2×500 mL), brine (200 mL) dried over Na₂SO₄, filtered and the solvent removed under vacuum. The residue was purified by MPLC on silica gel with gradient from 0% EtOAc/hexanes to 50% EtOAc/hexanes then 50% EtOAc/hexanes to afford the title compound. m/z (ES) 702 (MH-OAc)⁺, 784 (M+Na)⁺.

### Step B: Preparation of (1S)-3-[(2S,3R)-2-(4-{5-(acetyloxy)-2,2-dimethyl-1,3-dioxan-5-yl}ethynyl}phenyl)-1-{4-[3-(benzyloxy)prop-1-yn-1-yl]-phenyl}-4-oxoazetidin-3-yl)-1-(4-fluorophenyl)propyl acetate

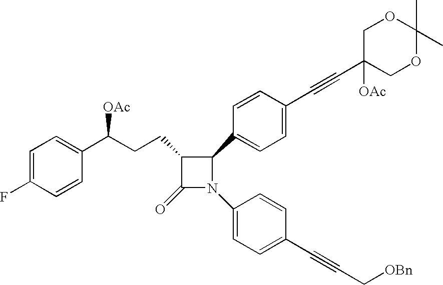

The title compound was prepared from the intermediate of step A according to the procedure for Example 1, step G. m/z (ES) 698 (MH-OAc)⁺.

### Step C: Preparation of (1S)-3-[(2S,3R)-2-(4-{2-[5-(acetyloxy)-2,2-dimethyl-1,3-dioxan-5-yl}ethyl}phenyl)-1-[4-(3-hydroxypropyl)phenyl]-4-oxoazetidin-3-yl}-1-(4-fluorophenyl)propyl acetate

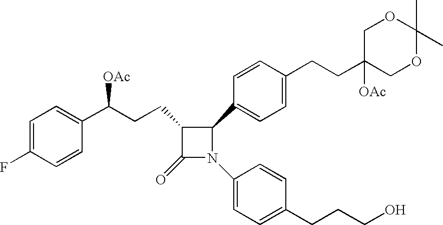

The title compound was prepared from the intermediate of step B according to the procedure for Example 1, step H. m/z (ES) 616 (MH-OAc)⁺. 698 (M+Na)⁺.

### Step D: Preparation of (1S)-3-[(2S,3R)-2-(4-{2-[5-(acetyloxy)-2,2-dimethyl-1,3-dioxan-5-yl}ethyl}phenyl)-4-oxo-1-[4-(3-oxopropyl)phenyl]-azetidin-3-yl}-1-(4-fluorophenyl)propyl acetate

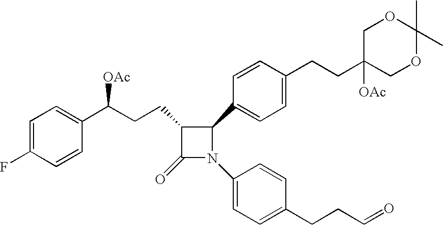

To a solution of the intermediate from Step C, Example 3 (75 mg, 0.11 mmol) in dichloromethane (1.5 mL) was added dropwise via syringe a 15% wt solution of Dess Martin reagent in dichloromethane (630 μL, 0.12 mmol) and the resulting mixture stirred at room temperature under nitrogen atmosphere for two hours. The mixture was then quenched with saturated sodium bicarbonate solution (2 mL) and extracted with dichloromethane (2×2 mL). The organics were combined, dried over sodium sulfate, filtered and then evaporated under vacuum. Preparative plate purification eluding with 60% ethyl acetate/40% hexane afforded the title compound. m/z (ES) 674 (MH)⁺.

### Step E: Preparation of (1S)-3-((2S,3R)-2-(4-{2-[5-(acetyloxy)-2,2-dimethyl-1,3-dioxan-5-yl}ethyl}phenyl)-4-oxo-1-{4-[3-(1,3-thiazol-2-ylamino)propyl]phenyl}-azetidin-3-yl)-1-(4-fluorophenyl)propyl acetate

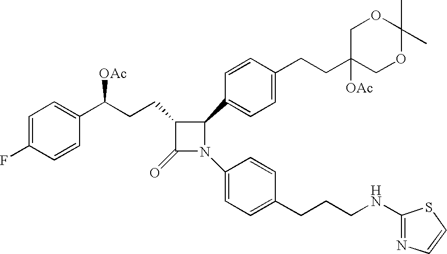

To a solution of the intermediate from Step D, Example 3 (15 mg, 0.02 mmol) in dichloromethane (0.5 mL) and acetic acid (10 μL) was added 2-aminothiazole (2 mg, 0.02 mmol) followed by 4A crushed molecular sieves and the resulting mixture stirred at room temperature under nitrogen atmosphere for eight hours. Sodium triacetoxyborohydride (12 mg, 0.06 mmol) was then added to the solution and the resulting suspension was stirred overnight at room temperature. The mixture was then quenched with saturated sodium bicarbonate solution (2 mL) and extracted with dichloromethane (2×5 mL). The organics were combined, dried over sodium sulfate, filtered and then evaporated under vacuum. Preparative plate purification eluding with 80% ethyl acetate/20% hexane afforded the title compound. m/z (ES) 758 (MH)⁺.

### Step F: Preparation of 3-[4-((2S,3R)-3-[(3S)-3-(acetyloxy)-3-(4-fluorophenyl)propyl]-4-oxo-1-{4-[3-(1,3-thiazol-2-ylamino)propyl]phenyl}azetidin-2-yl)phenyl]-1,1-bis(hydroxylmethyl)propyl acetate

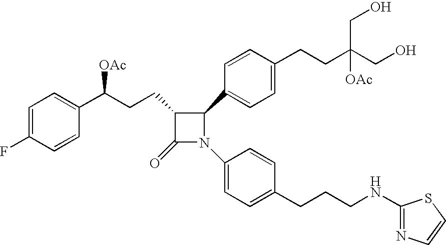

To a solution of the intermediate from Step E, Example 3 (4.0 mg, 0.005 mmol) in dichloromethane (0.5 mL) was added via syringe trifluoroacetic acid (0.2 mL) and the resulting solution stirred for 2 hours. The mixture was concentrated in vacuo and used without purification for the next reaction. m/z (ES) 718 (MH)+.

### Step G: Preparation of (3R,4S)-4-{4-[3,4-dihydroxy-3-(hydroxymethyl)butyl]phenyl}-3-[(3S)-3-(4-fluorophenyl)-3-hydroxypropyl]-1-{4-[3-(1,3-thiazol-2-ylamino)propyl]phenyl}azetidin-2-one

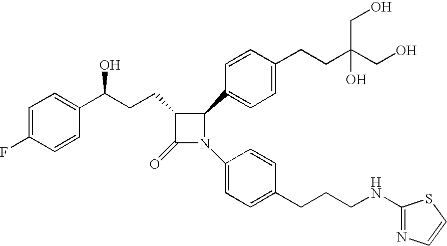

The title compound was prepared from the intermediate of step F, Example 3 according to the procedure for Example 1, step I. m/z (ES) 634 (M+H)⁺.

Using procedures similar to those described above the following Examples in Table 1 were prepared from the appropriate starting materials:

TABLE 1

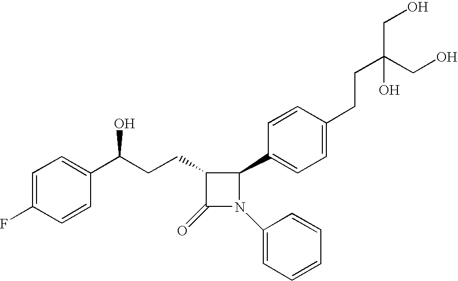

| | | | m/z (ES) |
|---|---|---|---|
| Example | R⁹ | MW | (M + H)⁺ |
| 4 | 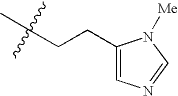 | 601.7 | 602 |
| 5 | 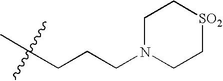 | 668.7 | 669 |
| 6 | 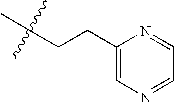 | 599.7 | 600 (50%) 582 (100%) —OH |
| 7 | 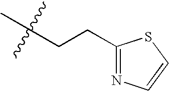 | 604.7 | 605 |
| 8 | 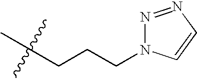 | 602.7 | 603 |
| 9 | 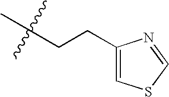 | 604.6 | 605 |
| 10 | 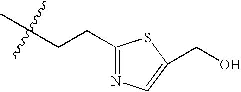 | 634.8 | 635 |
| 11 | 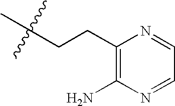 | 614.7 | 615 |
| 12 | 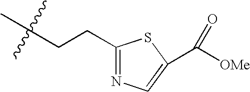 | 662.7 | 663 |
| 13 | 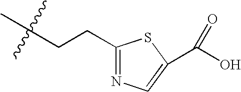 | 648.6 | 649 671 (MNa)⁺ |
| 14 | 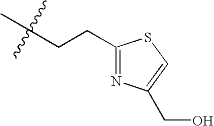 | 634.8 | 635 |
| 15 | 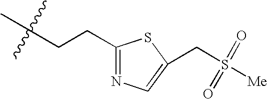 | 696.9 | 697 (50%) 679 (100%) —OH |
| 16 | 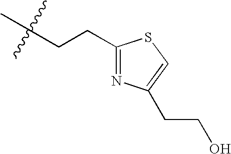 | 648.8 | 649 |
| 17 | 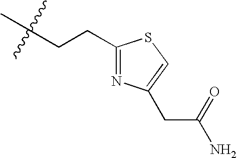 | 661.8 | 662 (80%) 644 (100%) —OH |
| 18 | 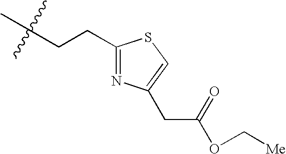 | 690.8 | 691 |
| 19 | 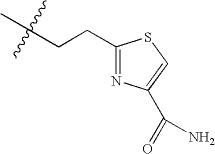 | 647.8 | 630 —OH |
| 20 | 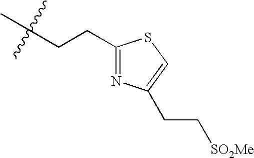 | 710.9 | 711 (40%) 693 (100%) —OH |
| 21 | 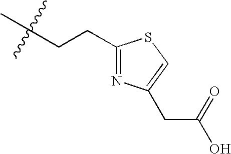 | 662.7 | 645 —OH |
| 22 | 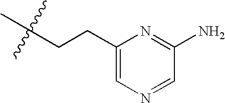 | 614.7 | 615 |
| 23 | 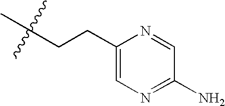 | 614.7 | 615 |
| 24 | 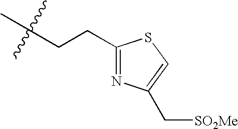 | 696.9 | 697 (30%) 679 (100%) —OH |
| 25 | 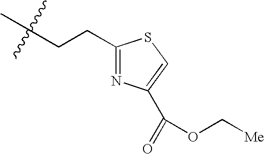 | 676.8 | 677 |
| 26 | 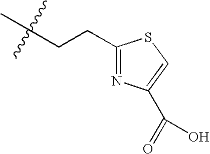 | 648.7 | 631 —OH |
| 27 | 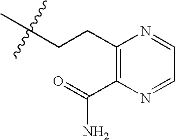 | 642.7 | 625 —OH |
| 29 | 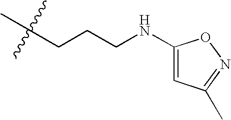 | 631.5 | 632 |
| 30 | 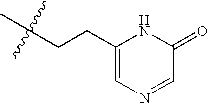 | 615.5 | 616 |
| 31 | 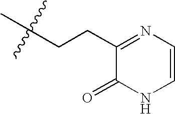 | 615.5 | 616 |
| 32 | 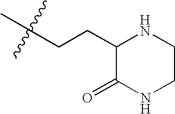 | 619.5 | 620 |
| 33 | 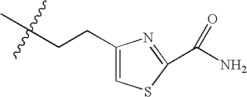 | 647.6 | 648 (40%) 630 (100%) —OH |
| 34 | 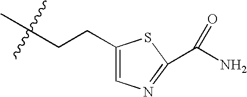 | 647.6 | 648 (30%) 630 (100%) —OH |
| 35 | 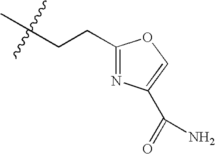 | 631.7 | 632 (30%) 614 (100%) —OH |
| 36 | 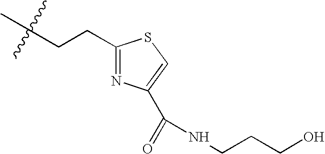 | 705.8 | 706 (80%) 688 (100%) —OH |
| 37 | 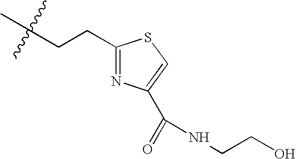 | 691.8 | 692 (50%) 674 (100%) —OH |
| 38 | 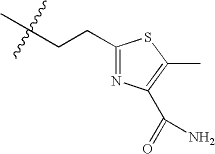 | 661.8 | 662 (60%) 644 (100%) —OH |
| 39 | 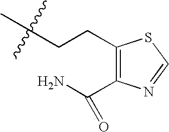 | 647.8 | 648 (60%) 630 (100%) —OH |
| 40 | 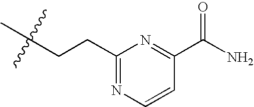 | 642.7 | 643 (40%) 626 (100%) —OH |
| 41 | 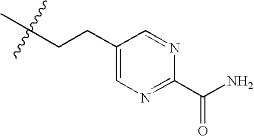 | 642.7 | 643 (25%) 626 (100%) —OH |
| 42 | 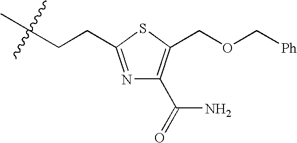 | 767.9 | 768 |
| 43 | 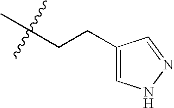 | 587 | 588 |

Compounds related to those described above having varying alkyl chain lengths linking the heterocycle of R⁹ to the rest of the structure may be prepared from the appropriate starting materials using the procedures described above.

### Example 44

### (3R,4S)-3-[(3S)-3-(4-fluorophenyl)-3-hydroxypropyl]-4-{4-[2,3,4,5-tetrahydroxy-4-(hydroxymethyl)pentyl]phenyl}-1-{4-[2-(1H-1,2,4-triazol-3-yl)ethyl]phenyl}azetidin-2-one

### Step A: Preparation of (1S)-3-[(2S,3R)-2-(4-allylphenyl)-4-oxo-1-(4{[(trifluoromethyl)sulfonyl]oxy}phenyl)azetidin-3-yl]-1-(4-fluorophenyl)propyl acetate

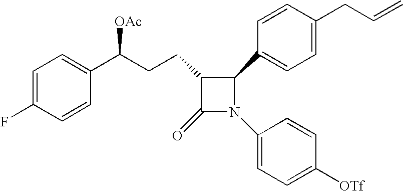

To a solution of (1S)-1-(4-fluorophenyl)-3-[(2S,3R)-2-(4-iodophenyl)-4-oxo-1-(4-{[(trifluoromethyl)sulfonyl]oxy}phenypazetidin-3-yl]propyl acetate (1.5 g, 2.17 mmol, see Example 1, step E) in anhydrous dioxane (25 mL) was added lithium chloride (275 mg, 6.50 mmol) and palladium tetrakis (255 mg, 0.22 mmol) and the resulting solution set under nitrogen atmosphere. Allyl tributyltin (780 μl, 2.60 mmol) was then added to the solution via syringe and the resulting mixture was heated to 80° C. for 16 hours. After cooling to room temperature, the solution was evaporate in vacuo and the residue was dissolved in ethyl acetate (100 mL). The organics were washed with water (50 mL), brine (50 mL), dried over magnesium sulfate, filtered, and evaporated in vacuo. MPLC purification using the Horizon instrument with a gradient eluant of 0-60% ethyl acetate in hexane afforded the title compound. m/z (ES) 546 (M-OAc)⁺ and 606 (M+H)⁺.

### Step B: Preparation of 2-[(acetyloxy)methyl]-2-hydroxybut-3-en-1-yl acetate

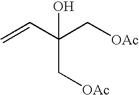

To a dry 100 mL round bottom flask set under nitrogen atmosphere was charged 2-oxopropane-1,3-diyl diacetate (10 g, 57.4 mmol) in 20 mL dry THF and cooled to 0° C. using an ice/water bath. To this cooled solution was added a 1.0M solution of vinylmagnesium bromide in THF (57.4 mL, 57.4 mmol) and the resulting solution stirred at 0° C. for 1 hour. The ice bath was removed and the resulting reaction mixture was stirred at ambient temperature for an additional 1.5 hrs. The reaction mixture was quenched with sat. aq. NH₄Cl (50 mL) and then extracted with ethyl acetate (100 mL). The organic layer was dried over Na₂SO₄, filtered and the solvent removed under vacuum to afford the crude intermediate. Horizon MPLC purification with a gradient eluant of 10-60% ethyl acetate in hexane afforded the title compound.

¹HNMR (500 MHz, CDCl₃): 5.86 (dd, J=11.0, 17.1 Hz, 1H), 5.47 (dd, J=0.8, 17.2 Hz, 1H), 5.30 (dd, J=0.8, 11.0 Hz, 1H), 4.1 (ABx q, J=11.4 Hz, 4H), 2.08 (s, 6H).

### Step C: Preparation of (1S)-3-[(2S,3R)-2-(4-{(2E)-5-(acetyloxy)-4-[(acetyloxy)methyl]-4-hydroxypent-2-en-1-yl}phenyl)-4-oxo-1-(4-{[(trifluoromethyl)sulfonyl]oxy}phenyl)azetidin-3-yl]-1-(4-fluorophenyl)propyl acetate

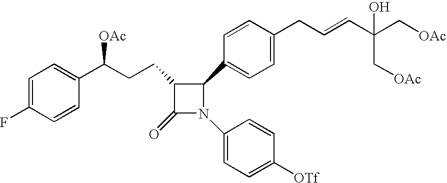

To a solution of (1S)-3-[(2S,3R)-2-(4-allylphenyl)-4-oxo-1-(4{[(triflo\uoromethyl)sulfonyl]oxy}phenyl)azetidin-3-yl]-1-(4-fluorophenyl)propyl acetate (510 mg, 0.84 mmol, intermediate Step A,) and 2-[(acetyloxy)methyl]-2-hydroxybut-3-en-1-yl acetate (205 mg, 1.02 mmol, intermediate Step B,) in anhydrous dichloromethane (5 mL) under nitrogen atmosphere was added Zhan catalyst I (670 mg, 1.02 mmol) and the resulting mixture stirred at room temperature for two hours. The reaction mixture was then evaporated in vacuo. Preparative plate purification eluting with 40% ethyl acetate/60% hexane afforded the title compound. m/z (ES) 780 (MH)⁺; 720 (M-OAc)⁺.

### Step D: Preparation of (1S)-3-[(2S,3R)-2-(4-{5-(acetyloxy)-4-[(acetyloxy)methyl]-2,3,4-trihydroxypentyl}phenyl)-4-oxo-1-(4-{[(trifluoromethyl)sulfonyl]oxy}phenyl)azetidin-3-yl]-1-(4-fluorophenyl)propyl acetate

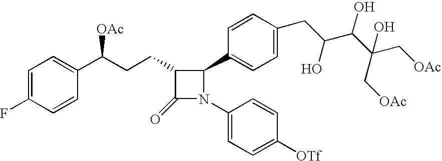

To a solution of (1S)-3-[(2S,3R)-2-(4-{(2E)-5-(acetyloxy)-4-[(acetyloxy)methyl]-4-hydroxypent-2-en-1-yl}phenyl)-4-oxo-1-(4-{[(trifluoromethyl)sulfonyl]oxy}phenyl)azetidin-3-yl]-1-(4-fluorophenyl)propyl acetate (200 mg, 0.26 mmol, intermediate Step C) in an 8:1 solution of acetone:water (4.5 mL) was added N-methylmorpholine-N-oxide (52 mg, 0.52 mmol) followed by a 2.5% wt solution of osmium tetraoxide in isopropanol (228 μL, 0.002 mmol) and the resulting mixture stirred at room temperature for 3 hours. The mixture was diluted with dichloromethane (20 mL) and washed with 1N HCl (15 mL), followed by brine (15 mL). The organics were dried over magnesium sulfate, filtered, and concentrated. Preparative plate purification eluting with 60% ethyl acetate/40% hexane afforded the title compound. m/z (ES) 814 (MH)⁺; 754 (M-OAc)⁺.

### Step E: Preparation of (1S)-3-[(2S,3R)-2-(4-{5-(acetyloxy)-4-[(acetyloxy)methyl]-2,3,4-trihydroxypentyl}phenyl)-4-oxo-1-(4-{[(1-trityl-1H-1,2,4-triazol-3-yl)ethynyl]phenyl)azetidin-3-yl]-1-(4-fluorophenyl)propyl acetate

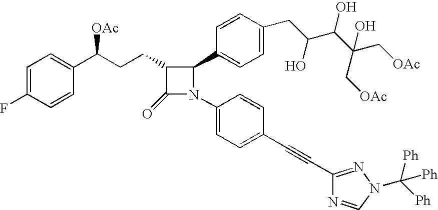

The title compound was prepared from (1S)-3-[(2S,3R)-2-(4-{5-(acetyloxy)-4-[(acetyloxy)methyl]-2,3,4-trihydroxypentyl}phenyl)-4-oxo-1-(4-{[(trifluoromethyl)sulfonyl]oxy}phenyl)azetidin-3-yl]-1-(4-fluorophenyl)propyl acetate (intermediate step D) and i-10 according to the procedure for Example 1, step G. m/z (ES) 999 (MH⁺)⁺, 757 (MH-trityl)⁺.

### Step F: Preparation of (1S)-3-{(2S,3R)-2-(4-{5-(acetyloxy)-4-[(acetyloxy)methyl]-2,3,4-trihydroxypentyl}phenyl)-4-oxo-1-[4-(1H-1,2,4-triazol-3-yl)ethynyl)phenyl]azetidin-3-yl]}-1-(4-fluorophenyl)propyl acetate

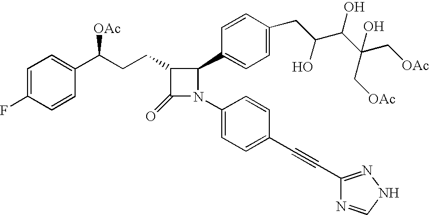

The title compound was prepared from (1S)-3-[(2S,3R)-2-(4-{5-(acetyloxy)-4-[(acetyloxy)methyl]-2,3,4-trihydroxypentyl}phenyl)-4-oxo-1-(4-{[(1-trityl-1H-1,2,4-triazol-3-yl)ethynyl]phenyl)azetidin-3-yl]-1-(4-fluorophenyl)propyl acetate (intermediate step E) according to the procedure for Example 2, step D. m/z (ES) 757 (MH)⁺.

### Step G: Preparation of (1S)-3-((2S,3R)-2-(4-{5-(acetyloxy)-4-[(acetyloxy)methyl]-2,3,4-trihydroxypentyl}phenyl)-4-oxo-1-{4-[2-(1H-1,2,4-triazol-3-yl)ethyl]phenyl}azetidin-3-yl]}-1-(4-fluorophenyl)propyl acetate

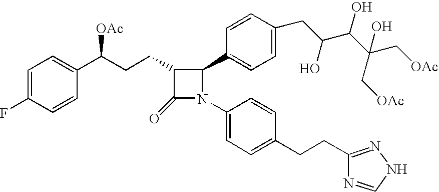

The title compound was prepared from (1S)-3-{(2S,3R)-2-(4-{5-(acetyloxy)-4-[(acetyloxy)methyl]-2,3,4-trihydroxypentyl}phenyl)-4-oxo-1-[4-(1H-1,2,4-triazol-3-yl)ethynyl)phenyl]azetidin-3-yl]}-1-(4-fluorophenyl)propyl acetate (intermediate step F) according to the procedure for Example 2, step E. m/z (ES) 761 (MH)⁺.

### Step H: Preparation of (3R,4S)-3-[(3S)-3-(4-fluorophenyl)-3-hydroxypropyl]-4-{4-[2,3,4,5-tetrahydroxy-4-(hydroxymethyl)pentyl]phenyl}-1-{4-[2-(1H-1,2,4-triazol-3-yl)ethyl]phenyl}azetidin-2-one

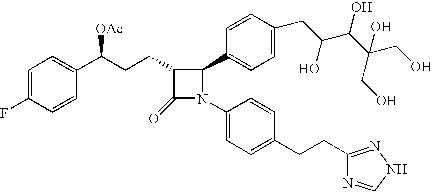

The title compound was prepared from (1S)-3-((2S,3R)-2-(4-{5-(acetyloxy)-4-[(acetyloxy)methyl]-2,3,4-trihydroxypentyl}phenyl)-4-oxo-1-{4-[2-(1H-1,2,4-triazol-3-yl)ethyl]phenyl}azetidin-3-yl]}-1-(4-fluorophenyl)propyl acetate (intermediate step G) according to the procedure for Example 2, step F. m/z (ES) 635 (MH)⁺.

### Example 45

### (3R,4S)-3-[(3S)-3-(4-fluorophenyl)-3-hydroxypropyl]-4-{4-[1,2,3,4-tetrahydroxy-3-(hydroxymethyl)butyl]phenyl}-1-{4-[2-(1H-1,2,4-triazol-3-yl)ethyl]phenyl}azetidin-2-one

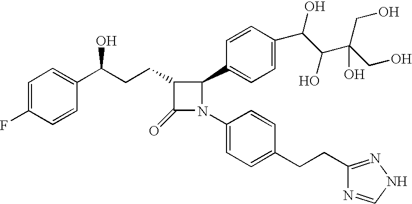

The title compound was prepared from the appropriate starting materials using procedures similar to those described above in the prior Example 44, but substituting vinyl tributyltin for the allyl tributyltin used therein. m/z (ES) 621 (MH)⁺.

### Example 46

### Step A: Preparation of (1S)-3-((2S,3R)-2-(4-{(2E)-5-(acetyloxy)-4-[(acetyloxy)methyl]-4-hydroxypent-2-en-1-yl}phenyl)-4-oxo-1-(4-[(1-trityl-1H-1,2,4-triazol-3-yl)ethynyl]phenyl)azetidin-3-yl]-1-(4-fluorophenyl)propyl acetate

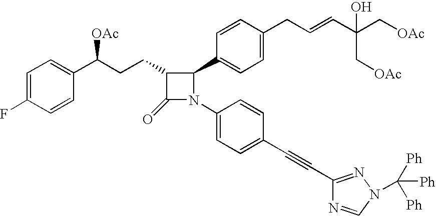

The title compound was prepared from (1S)-3-[(2S,3R)-2-(4-{(2E)-5-(acetyloxy)-4-[(acetyloxy)methyl]-4-hydroxypent-2-en-1-yl}phenyl)-4-oxo-1-(4-{[(trifluoromethyl)sulfonyl]oxy}phenyl)azetidin-3-yl]-1-(4-fluorophenyl)propyl acetate (Example 44, Step C) and i-10 according to the procedure for Example 1, step G. m/z (ES) 965 (MH)⁺, 723 (MH-trityl)⁺.

### Step B: Preparation of 1(S)-3-((2S,3R)-2-(4-{5-(acetyloxy)-4-[(acetyloxy)methyl]-4-hydroxypentyl)phenyl)-4-oxo-1-{4-[2-(1H-1,2,4-triazol-3-yl)ethyl]phenyl}azetidin-3-yl)-1-(4-fluorophenyl)propyl acetate

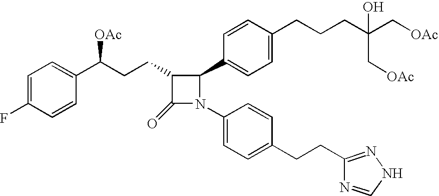

To a suspension of 10% palladium on carbon (40 mg) in ethyl acetate/ethanol (3/1; 2 mL) was added a solution of (1S)-3-((2S,3R)-2-(4-{(2E)-5-(acetyloxy)-4-[(acetyloxy)methyl]-4-hydroxypent-2-en-1-yl}phenyl)-4-oxo-1-(4-[(1-trityl-1H-1,2,4-triazol-3-yl)ethynyl]phenyl)azetidin-3-yl]-1-(4-fluorophenyl)propyl acetate (22 mg, 0.03 mmol, intermediate step A) in ethanol (0.2 mL) and the resulting mixture set under hydrogen atmosphere and stirred overnight. The catalyst was filtered off using a Gilmen PTFE 0.45 μM syringe filter disc and washed with ethanol (10 mL). The organics were concentrated to dryness to afford the crude product. The compound was used in the next reaction with further purification. m/z (ES) 729 (MH)⁺.

### Step C: Preparation of (3R,4S)-4-{4-[4,5-dihydroxy-4-(hydroxymethyl)pentyl]phenyl}-3-[(3S)-3-(4-fluorophenyl)-3-hydroxpropyl]-1-{4-[2-(1H-1,2,4-triazol-3-yl)ethyl]phenyl)azetidin-2-one

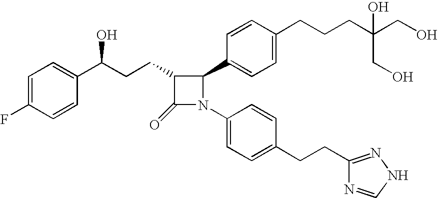

The title compound was prepared from 1(S)-3-((2S,3R)-2-(4-{5-(acetyloxy)-4-[(acetyloxy)methyl]-4-hydroxypentyl)phenyl)-4-oxo-1-{4-[2-(1H-1,2,4-triazol-3-yl)ethyl]phenyl}azetidin-3-yl)-1-(4-fluorophenyl)propyl acetate (intermediate step C) according to the procedure for Example 2, step F. m/z (ES) 603 (MH)⁺.

### Example 47

### Step A: Preparation of 5-ethynyl-2,2,2′,2′-tetramethyl-4,5′-bi-1,3-dioxane-5,5′-diol

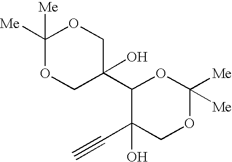

To a dry 100 mL round bottom flask set under nitrogen atmosphere was charged 2,2-dimethyl-1,3-dioxane-5-one (5 g, 38.4 mmol) in 20 mL dry THF and cooled to 0° C. using an ice/water bath. To this cooled solution was added a 0.5M solution of ethynylmagnesium bromide in THF (76.8 mL, 38.4 mmol) and the resulting solution stirred at 0° C. for 30 minutes. The ice bath was removed and the resulting reaction mixture was stirred at ambient temperature for an additional 1.5 hrs. The reaction mixture was quenched with sat. aq. NH₄Cl (50 mL) and then extracted with ethyl acetate (100 mL). The organic layer was dried over Na₂SO₄, filtered and the solvent removed under vacuum to afford the crude intermediate. Horizon MPLC purification with a gradient eluant of 10-60% ethyl acetate in hexane afforded the title compound. ¹HNMR (500 MHz, CDCl₃) δ: 4.63 (s, 1H), 4.32 (d, J=12.2 Hz, 1H), 4.08 (dd, J=1.2, 12.2 Hz, 1H), 4.04 (s, 1H), 3.98 (app t, J=12.2 Hz, 2H), 3.82 (d, J=12.1H, 1H) 3.78 (dd, J=1.2, 12.2 Hz, 1H), 3.66 (s, 1H), 2.69 (s, 1H), 1.51 (s, 3H), 1.50 (s, 3H), 1.48 (s, 3H), 1.47 (s, 3H).

### Step B: Preparation of (1S)-3-[(2S,3R)-2-{4-[(5,5′-dihydroxy-2,2,2′,2′-tetramethyl-4,5′-bi-1,3-diox-5-yl)ethynyl]phenyl}-4-oxo-1-(4-{[(trifluoromethyl)sulfonyl]-oxy}phenyl)azetidin-3-yl]-1-(4-fluorophenyl)propyl acetate

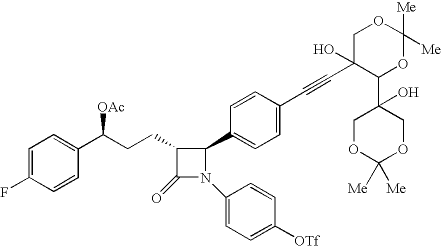

The title compound was synthesized from 5-ethynyl-2,2,2′,2′-tetramethyl-4,5′-bi-1,3-dioxane-5,5′-diol (intermediate step A) and (1S)-1-(4-fluorophenyl)-3-[(2S,3R)-2-(4-iodophenyl)-4-oxo-1-(4-{[(trifluoromethyl)sulfonyl]oxy}phenyl)azetidin-3-yl]propyl acetate (intermediate, Example 1, Step E) according to the procedure for Example 1, step F. m/z (ES) 850 (MH)⁺.

### Step C: Preparation of (1S)-3-[(2S,3R)-2-{4-[(5,5′-dihydroxy-2,2,2′,2′-tetramethyl-4,5′-bi-1,3-diox-5-yl)ethynyl]phenyl}-4-oxo-1-{4-[(1-trityl-1H-1,2,4-triazol-3-yl)ethynyl]phenyl}azetidin-3-yl]-1-(4-fluorophenyl)propyl acetate

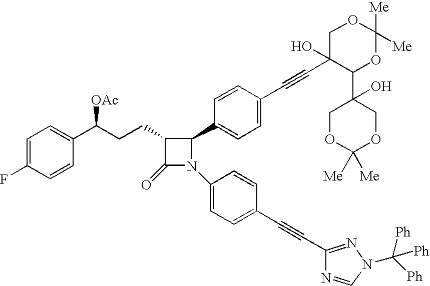

The title compound was prepared from (1S)-3-[(2S,3R)-2-{4-[(5,5′-dihydroxy-2,2,2′,2′-tetramethyl-4,5′-bi-1,3-diox-5-yl)ethynyl]phenyl}-4-oxo-1-(4-{[(trifluoromethyl)sulfonyl]oxy}-phenyl)azetidin-3-yl]-1-(4-fluorophenyl)propyl acetate (intermediate step B) and i-10 according to the procedure for Example 1, step G. m/z (ES) 1035 (MH)⁺, 793 (MH-trityl)⁺.

### Step D: Preparation of (1S)-3-[(2S,3R)-2-{4-[(5,5′-dihydroxy-2,2,2′,2′-tetramethyl-4,5′-bi-1,3-diox-5-yl)ethyl]phenyl}-4-oxo-1-{4-[2-(1H-1,2,4-triazol-3-yl)ethyl]phenyl}azetidin-3-yl]-1-(4-fluorophenyl)propyl acetate

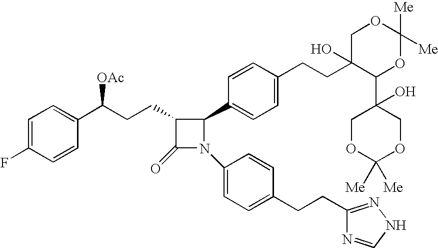

The title compound was prepared from (1S)-3-[(2S,3R)-2-{4-[(5,5′-dihydroxy-2,2,2′,2′-tetramethyl-4,5′-bi-1,3-diox-5-yl)ethynyl]phenyl}-4-oxo-1-{4-[(1-trityl-1H-1,2,4-triazol-3-yl)ethynyl]phenyl}azetidin-3-yl]-1-(4-fluorophenyl)propyl acetate (intermediate step C) according to the procedure for Example 46, step B. m/z (ES) 801 (MH)⁺. ¹HNMR (500 MHz, CDCl₃) δ: 7.98 (s, 1H), 7.32-7.28 (m, 2H), 7.24-7.18 (m, 2H), 7.17 (d, J=8.5 Hz, 2H), 7.06-7.00 (m, 3H), 5.72 (t, J=6.7 Hz, 1H), 4.58 (d, J=1.8 Hz, 1H), 4.15 (dd, J=7.8, 12.6 Hz, 2H), 3.97 (d, J=12.6 Hz, 1H), 3.85 (d, J=11.7 Hz, 1H), 3.8 (d, 4.0 Hz, 1H), 3.60 (dd, J=2.4 Hz, 11.7 Hz, 1H), 3.52 (s, 1H), 3.50 (d, J=12.8, 1H), 3.42 (br s, 1H), 3.10-2.98 (m, 4H), 2.80-2.70 (m, 1H), 2.72-2.64 (m, 1H), 2.12-2.00 (m, 3H), 2.08 (s, 3H), 1.92-1.84 (m, 2H), 1.48 (s, 3H), 1.45 (s, 3H), 1.43 (overlapping singlets, 6H).

### Step E: Preparation of (1S)-1-(4-fluorophenyl)-3-((3R,4S)-2-oxo-4-{4-[3,4,5,6-tetrahydroxy-3,5-bis(hydroxymethyl)hexyl]phenyl}-1-{4-[2-(1H-1,2,4-triazol-3-yl)ethyl]phenyl}azetidin-3-yl)propyl acetate

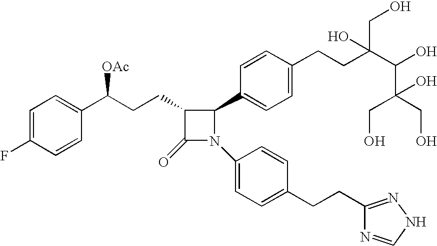

To a solution of (1S)-3-[(2S,3R)-2-{4-[(5,5′-dihydroxy-2,2,2′, 2′-tetramethyl-4,5′-bi-1,3-diox-5-yl)ethyl]phenyl}-4-oxo-1-{4-[2-(1H-1,2,4-triazol-3-yl)ethyl]phenyl}azetidin-3-yl]-1-(4-fluorophenyl)propyl acetate (intermediate Step D) in THF/water (10/1; 1.65 mL) was added trifluoroacetic acid (0.3 mL) and the resulting solution stirred at room temperature for 3 hours. Evaporate in vacuo and azeotrope with toluene (3×5 mL) to remove traces of water and ecess TFA. The residue was used for the next reaction without further purification. m/z (ES) 721 (MH)⁺.

### Step F: Preparation of (3R,4S)-3-[(3S)-3-(4-fluorophenyl)-3-hydroxypropyl]-4-{4-[3,4,5,6-tetrahydroy-3,5-bis(hydroxymethyl)hexyl]phenyl}-1-{4-[2-(1H-1,2,4-triazol-3-yl)ethyl]phenyl}azetidin-2-one

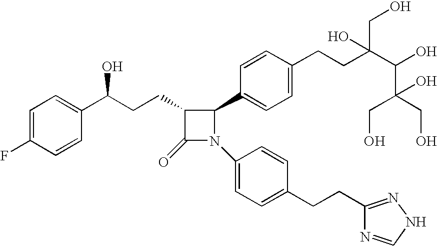

The title compound was prepared from (15)-1-(4-fluorophenyl)-3-((3R,4S)-2-oxo-4-{4-[3,4,5,6-tetrahydroxy-3,5-bis(hydroxymethyl)hexyl]phenyl}-1-{4-[2-(1H-1,2,4-triazol-3-yl)ethyl]phenyl}azetidin-3-yl)propyl acetate (intermediate step E) according to the procedure from Example 2, step F. m/z (ES) 679 (MH)⁺.

### Example 48

### Step A: Preparation of (2R,3S,5R,6S)-2,3,4,5,6-pentakis(benzyloxy)-1-ethynylcyclohexanol

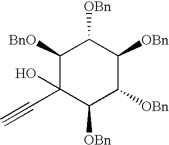

To a dry 100 mL round bottom flask was charged with a 0.5M solution of ethynylmagnesium bromide in THF (1.0 mL, 0.50 mmol) under nitrogen atmosphere. The resulting solution was cooled to 0° C. in an ice bath. To the cooled solution was added slowly a solution of (2R,3S,5R,6S)-2,3,4,5,6 pentakis(benzyloxy)cyclohexanone (300 mg, 0.48 mmol) in 0.5 mL dry THF. The ice bath was removed and the resulting reaction mixture was stirred at ambient temperature for 1.5 hrs. The reaction mixture was quenched with sat. aq. NH₄Cl (50 mL) and then extracted with ethyl acetate (100 mL). The organic layer was dried over Na₂SO₄, filtered and the solvent removed under vacuum. Preparative plate purification eluting with 20% ethyl acetate/80% hexane afforded the title compound.

(2R,3S,5R,6S)-2,3,4,5,6 Pentakis(benzyloxy)cyclohexanone can be prepared from myo-inosose-2 following the procedures described in Posternak, T, in E. G. Ball (editor), Biochemical Preparations, Vol II, John Wiley and Sons, Inc, New York, p. 57 (1952), and Billington D. C., Baker R, Kulagowski J J, Mawer I. M., J. Chem Soc Chem Comm (4), p. 314-316 (1987).

### Step B: Preparation of (1S)-1-(4-fluorophenyl)-3-[(3R,4S)-2-oxo-4-(4-{[2R,3S,5R,6S)-2,3,4,5,6-pentakis(benzyloxy)-1-hydroxycyclohexyl]ethynyl]phenyl)-1-(4-{[(trifluoromethyl)sulfonyl]oxy}phenyl)azetidin-3-yl]propyl acetate

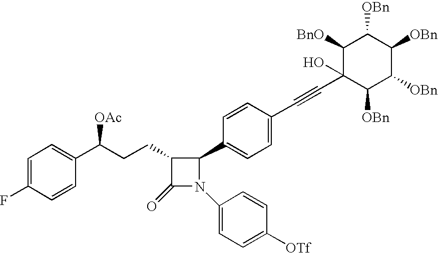

The title compound was synthesized from (2R,3S,5R,6S)-2,3,4,5,6-pentakis(benzyloxy)-1-ethynylcyclohexanol (intermediate Step A) and (15)-1-(4-fluorophenyl)-3-[(2S,3R)-2-(4-iodophenyl)-4-oxo 1-(4 {[(trifluoromethyl)sulfonyl]oxy}phenyl)azetidin-3-yl]propyl acetate (intermediate, Example 1, Step E) according to the procedure for Example 1, step F. m/z (ES) 1218 (MH)⁺.

### Step C: Preparation of (1S)-1-(4-fluorophenyl)-3-((3R,4S)-2-oxo-4-(4-{[2R,3S,5R,6S)-2,3,4,5,6-pentakis(benzyloxy)-1-hydroxycyclohexyl]ethynyl}phenyl)-1-{4-[(1-trityl-1H-1,2,4-triazol-3-ynethynyl]phenyl}azetidin-3-yl]propyl acetate

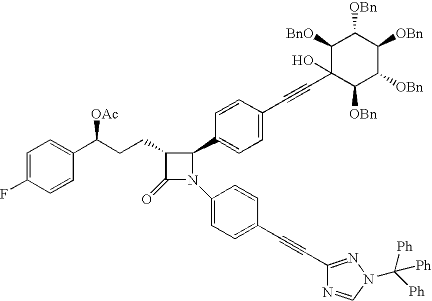

The title compound was prepared from intermediate of step B and i-10 according to the procedure for Example 1, step G. m/z (ES) 1403 (MH)⁺, 1160 (MH-trityl)⁺.

### Step D: Preparation of (1S)-1-(4-fluorophenyl)-3-((3R,4S)-2-oxo-4-(4-{2-[2R,3S,5R, 6S)-2,3,4,5,6-pentakis(benzyloxy)-1-hydroxycyclohexyl]ethyl}phenyl)-1-{4-[2-(1H-1,2,4-triazol-3-yl)ethyl]phenyl}azetidin-3-yl]propyl acetate

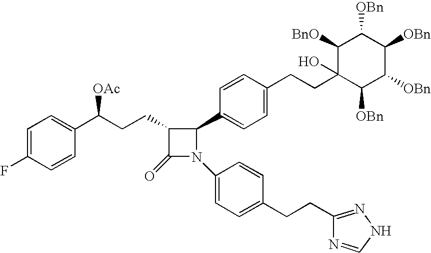

The title (major) compound was prepared from (1S)-1-(4-fluorophenyl)-3-((3R,4S)-2-oxo-4-(4-{[2R,3S,5R,6S)-2,3,4,5,6-pentakis(benzyloxy)-1-hydroxycyclohexyl]ethynyl}phenyl)-1-{4-[(1-trityl-1H-1,2,4-triazol-3-yl)ethynyl]phenyl}azetidin-3-yl]propyl acetate (intermediate Step C) according to the procedure for Example 46, Step B. m/z (ES) 1169 (MH)⁺. A mixture of the 4 other products were seen in minor percentages. They were identified via LC-MS as the mono-[m/z (ES) 809, 5%], di-[m/z (ES) 899, 15%], and tri-benzyl [m/z (ES) 989, 25%] protected hydroxyl compounds; along with a trace of the completely de-benzylated product [m/z (ES) 719, ±1%]. The mixture was used in the next reaction.

### Step E: Preparation of (1S)-1-(4-fluorophenyl)-3-((2R,3S)-2-(4-{2-[2R,3S,4S,5R,6S)-1,2,3,4,5-6-hexahydroxycyclohexyl]ethyl}phenyl)-4-oxo-1-{4-[2-(1H-1,2,4-triazol-3-yl)ethyl]phenyl}azetidin-3-yl]propyl acetate

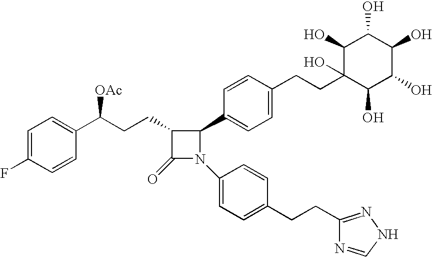

H-cube hydrogenation: the H-cube was set for 10 bar hydrogen gas with eluant follow of 1.0 mL/minute of ethanol. A solution of the intermediate from step D (mixture 42 mg) in ethanol 15 mL was then prepared and passed through the 10% palladium on carbon cartridge of the H-cube. After 20 mL of ethanol had passed through, the hydrogen was shut down and the column heated to 50° C. Another 20 mL of ethanol was then passed through the column to wash all the compound of the catalyst cartridge. The product was observed in the later fractions that come from the H-cube during the second 20 mL ethanol wash. These fractions were combined and concentrated to dryness. Gilson HPLC purification with a gradient eluent of 10-70% acetonitrile/water (0.1% TFA buffer) afforded the title compound. m/z (ES) 719 (MH)⁺.

### Step F: Preparation of (3R,4S)-3-{(3S)-3-(4-fluorophenyl)-3-hydroxypropyl]-4-(4-{2-[(2R,3S,4S,5R,6S)-1,2,3,4,5,6-hexahydroxycyclohexyl]ethyl}phenyl)-1-{4-[2-(1H-1,2,4-triazol-3-yl)ethyl]phenyl}azetidin-2-one

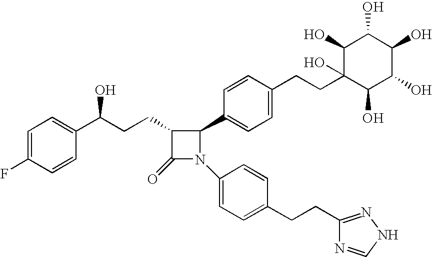

The title compound was prepared from (1S)-1-(4-fluorophenyl)-3-((2R,3S)-2-(4-{2-[2R,3S,4S,5R,6S)-1,2,3,4,5-6-hexahydroxycyclohexyl]ethyl}phenyl)-4-oxo-1-{4-[2-(1H-1,2,4-triazol-3-yl)ethyl]phenyl}azetidin-3-yl]propyl acetate (intermediate step E) according to the procedure for Example 2, step F. m/z (ES) 677 (MH)⁺.

### Example 49

### Step A: Preparation of 2-hydroxy-5-iodobenzaldehyde

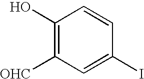

To a solution of salicylaldehyde (10.65 ml, 100 mmol) in anhydrous CH₂Cl₂ (40 ml) cooled at 0° C. was added iodime iodine monochloride (100 ml of a 1.0M solution in CH₂Cl₂, 100 mmol). After complete addition the mixture was allowed to warm to room temperature and stirred for 16 hours. The reaction was quenched by the addition of 10% aqueous Na₂SO₃ (150 ml). The organic layer was separated, washed with water (200 ml), dried over MgSO₄, filtered and evaporated. The residue was recrystallised from cyclohexane to afford the title compound. ¹HNMR (500 MHz, CDCl₃) δ: 9.85 (s, 1H), 7.86 (d, J=2.3, 1H), 7.78 (dd, and 2.3, 1H), 6.82 (d, J=8.7, 1H).

### Step B: Preparation of 2-benzyloxy-5-iodobenzaldehyde

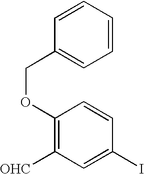

To a solution of 2-hydroxy-5-iodobenzaldehyde (10.4 g, 41.9 mmol; intermediate of step A) in anhydrous THF (100 ml) was added portionwise sodium hydride (1.85 g of a 60% suspension in oil, 46.1 mmol). After complete addition the mixture was stirred for 15 mins then benzyl bromide (5.48 ml, 46.1 mmol) added and the resulting mixture stirred at room temperature for 3 days. The mixture was poured into water (250 ml) and extracted with EtOAc (3×100 ml). The combined EtOAc layers were washed with water (200 ml), sat. NaCl (100 ml), dried over Na₂SO₄, filtered and evaporated. The residue was triturated with a mixture of Et₂O and hexanes, filtered and dried to afford the title compound. ¹HNMR (500 MHz, CDCl₃) δ: 10.44 (s, 1H), 8.12 (d, J=2.3, 1H), 7.79 (dd, J=8.6 and 2.3), 7.44 (m, 5H), 6.86 (d, J=8.6, 1H), 5.19 (s, 2H).

### Step C: 4-{[(1E)-(3-benzyloxy-5-iodophenyl)methylene]amino}phenol

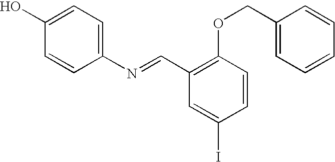

The title compound was prepared from 2-benzyloxy-5-iodobenzaldehyde (intermediate from step B) and 4-hydroxyaniline according to the procedure of Example 1, step A. ¹HNMR (500 MHz, CDCl₃) δ: 8.87 (s, 1H), 8.47 (d, J=2.3, 1H), 7.67 (dd, J=8.7 and 2.3, 1H), 7.44-7.35 (m, 5H), 7.19 (d, J=8.7, 2H), 6.87 (d, J=8.7, 2H), 6.79 (d, J=8.7, 1H).

### Step D: 4-[(2S,3R)-3-[(3S)-3-(acetyloxy)-3-(4-fluorophenyl)propyl]-2-(2-benzyloxy-5-iodophenyl)-4-oxoazetidin-1-yl]phenyl acetate

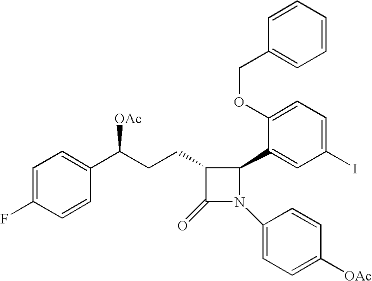

The title compound was prepared from 4-{[(1E)-(3-benzyloxy-5-iodophenyl)methylene]amino}phenol (intermediate from step C) and (4S)-3-[(5S)-5-(4-fluorophenyl)-5-hydroxypentanoyl]-4-phenyl-1,3-oxazolidin-2-one (prepared according to the procedures of Fu, X.; McCallister, T. L.; Thiruvengadam, T. K.; Tann, C. H.; and Su, D. Tetrahedron Lett. (2003) 44, 801-804) according to the procedure of Example 1, steps B, C, and D. ¹HNMR (500 MHz, CDCl₃) δ: 7.58 (dd, J=8.5 and 2.1, 1H), 7.47 (d, J=2.1, 1H), 7.42 (m, 3H), 7.36 (m, 2H), 7.25 (d, J=8.9, 2H), 7.15 (dd, J=8.5 and 5.2, 2H), 7.00 (d, J=8.7, 2H), 6.97 (t, J=8.7, 2H), 6.78 (d, J=8.7, 1H), 5.58 (t, J=6.6, 1H), 5.11 (q, J=13.8 and 11.6, 2H), 4.96 (d, J=2.6), 3.10 (m, 1H), 2.28 (s, 3H), 2.01 (s, 3H), 2.00-1.92 (m, 2H), 1.86-1.72 (m, 2H).

### Step E: Preparation of (1S)-1-(4-fluorophenyl)-3-[(2S,3R)-2-(2-benzyloxy-5-iodophenyl)-4-oxo-1-(4-{[(trifluoromethyl)sulfonyl]oxy}phenyl)azetidin-3-yl]propyl acetate

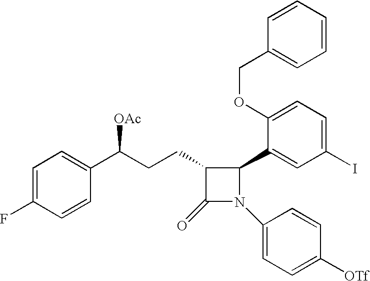

The title compound was prepared from 4-[(2S,3R)-3-[(3S)-3-(acetyloxy)-3-(4-fluorophenyl)propyl]-2-(2-benzyloxy-5-iodophenyl)-4-oxoazetidin-1-yl]phenyl acetate (intermediate from step D) according to the procedure of Example 1, step E. ¹HNMR (500 MHz, CDCl₃) δ: 7.61 (dd, J=8.5 and 2.1, 1H), 7.46 (d, J=2.1, 1H), 7.43 (m, 3H), 7.36 (m, 2H), 7.30 (d, J=8.9, 2H), 7.17 (m, 4H), 6.99 (t, J=8.7, 2H), 6.81 (d, J=8.7, 1H), 5.61 (t, J=6.6, 1H), 5.15 (d, J=11.7, 1H), 5.09 (d, J=11.7, 1H), 4.97 (d, J=2.5, 1H), 3.17 (m, 1H), 2.07 (s, 3H), 2.07-1.96 (m, 2H), 1.86-1.74 (m, 2H).

### Step F: Preparation of (1S)-3-[(2S,3R)-2-[5-{4-(acetyloxy)-3-[(acetyloxy)methyl]-3-hydroxybut-1-yn-1-yl}-2-(benzyloxy)phenyl]-4-oxo-1-(4-{[(trifluoromethyl)sulfonyl]oxy}phenyl)azetidin-3-yl]-1-(4-fluorophenyl)propyl acetate

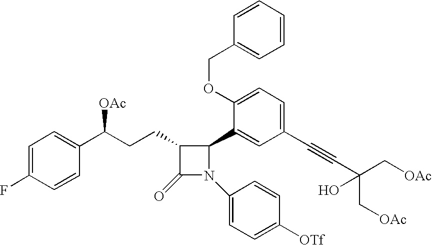

The title compound was prepared from (1S)-1-(4-fluorophenyl)-3-[(2S,3R)-2-(2-benzyloxy-5-iodophenyl)-4-oxo-1-(4-{[(trifluoromethyl)sulfonyl]oxy}phenyl)azetidin-3-yl]propyl acetate (intermediate from step E) and 2-ethynylpropane-1,2,3-triol 1,3-diacetate (intermediate i-2) according to the procedure of Example 1, step F. ¹HNMR (500 MHz, CDCl₃) δ: 7.44-7.39 (m, 4H), 7.37 (m, 2H), 7.31 (d. J=9.2, 2H), 7.26 (d, J=1.8, 1H), 7.19-7.14 (m, 4H), 7.01-6.96 (m, 3H), 5.59 (t, J=6.6, 1H), 5.18 (d, J=11.5, 1H), 5.12 (d, J=11.7, 1H), 5.01 (d, J=2.3, 1H), 4.33 (dd, J=11.4 and 4.1, 2H), 4.27 (dd, J=11.4 and 3.2, 2H), 3.14 (m, 1H), 2.09 (s, 6H), 2.02 (s, 3H), 2.00-1.94 (m, 2H), 1.86-1.74 (m, 2H).

### Step G: Preparation of (1S)-3-((2S,3R)-2-[5-{4-(acetyloxy)-3-[(acetyloxy)methyl]-3-hydroxybut-1-yn-1-yl}-2-(benzyloxy)phenyl]-4-oxo-1-{4-[(trimethylsilyl)ethynyl]phenyl)azetidin-3-yl)-1-(4-fluorophenyl)propyl acetate

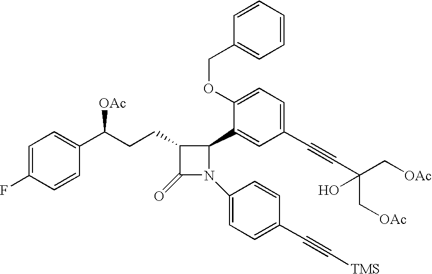

The title compound was prepared from (1S)-3-[(2S,3R)-2-[5-{4-(acetyloxy)-3-[(acetyloxy)methyl]-3-hydroxybut-1-yn-1-yl}-2-(benzyloxy)phenyl]-4-oxo-1-(4-{[(trifluoromethyl)sulfonyl]oxy}phenyl)azetidin-3-yl]-1-(4-fluorophenyl)propyl acetate (intermediate from step F) and trimethylsilylacetylene according to the procedure of Example 2, step A. ¹HNMR (500 MHz, CDCl₃) δ: 7.47-7.42 (m, 3H), 7.39-7.35 (m, 5H), 7.20 (d. J=1.8, 1H), 7.18-7.14 (m, 4H), 6.99-6.96 (m, 3H), 5.58 (t, J=6.7, 1H), 5.17 (d, J=11.4, 1H), 5.12 (d, J=11.4, 1H), 5.01 (d, J=2.1, 1H), 4.33 (d, J=11.4, 2H), 4.27 (d, J=11.4, 2H), 3.07 (m, 1H), 2.09 (s, 6H), 2.01 (s, 3H), 2.00-1.94 (m, 2H), 1.88-1.72 (m, 2H), 0.24 (s, 9H).

### Step H: Preparation of (1S)-3-[(2S,3R)-2-[5-{4-(acetyloxy)-3-[(acetyloxy)methyl]-3-hydroxybut-1-yn-1-yl}-2-(benzyloxy)phenyl]-1-(4-ethynylphenyl)-4-oxoazetidin-3-yl)-1-(4-fluorophenyl)propyl acetate

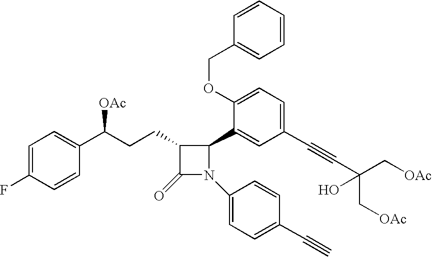

The title compound was prepared from (1S)-3-((2S,3R)-2-[5-{4-(acetyloxy)-3-[(acetyloxy)methyl]-3-hydroxybut-1-yn-1-yl}-2-(benzyloxy)phenyl]-4-oxo-1-{4-[(trimethylsilyl)ethynyl]phenyl)azetidin-3-yl)-1-(4-fluorophenyl)propyl acetate (intermediate from step G) according to the procedure of Example 2, step B. ¹HNMR (500 MHz, CDCl₃) δ: 7.45-7.43 (m, 3H), 7.40-7.37 (m, 5H), 7.23 (d. J=2.1, 1H), 7.19 (d, J=8.5, 2H), 7.15 (dd, J=8.7 and 5.5, 2H), 7.00-6.96 (m, 3H), 5.58 (t, J=6.6, 1H), 5.17 (d, J=11.5, 1H), 5.12 (d, J=11.5, 1H), 5.01 (d, J=2.3, 1H), 4.33 (d, J=11.3, 2H), 4.26 (d, J=11.3, 2H), 3.08 (m, 1H), 3.05 (s, 1H), 2.09 (s, 6H), 2.01 (s, 3H), 2.00-1.94 (m, 2H), 1.88-1.72 (m, 2H).

### Step I: Preparation of (1S)-3-[(2S,3R)-2-[5-{4-(acetyloxy)-3-[(acetyloxy)methyl]-3-hydroxybut-1-yn-1-yl}-2-(benzyloxy)phenyl]-1-(4-{[4-(aminocarbonyl)-1-3-thiazol-2-yl]ethylnyl}phenyl)-4-oxoazetidin-3-yl]-1-(4-fluorophenyl)propyl acetate

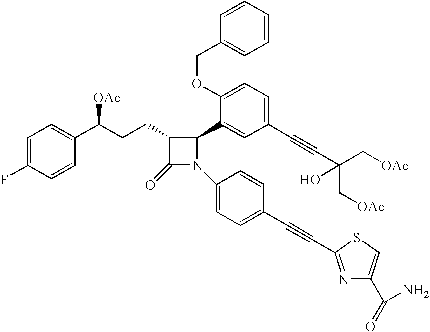

The title compound was prepared from (1S)-3-[(2S,3R)-2-[5-{4-(acetyloxy)-3-[(acetyloxy)methyl]-3-hydroxybut-1-yn-1-yl}-2-(benzyloxy)phenyl]-1-(4-ethynylphenyl)-4-oxoazetidin-3-yl)-1-(4-fluorophenyl)propyl acetate (intermediate from step H), and 2-bromothiazole-4-carboxamide (i-12) according to the procedure of Example 2, step C. ¹HNMR (500 MHz, CDCl₃) δ: 8.18 (s, 1H), 7.51 (d, J=8.7, 2H), 7.44-7.41 (m, 3H), 7.44-7.41 (m, 3H), 7.39-7.36 (m, 3H), 7.28-7.13 (m, 4H), 7.17-7.14 (m, 3H), 7.00-6.96 (m, 3H), 5.76 (br s, 1H), 5.58 (t, J=6.7, 1H), 5.18 (d, J=11.7, 1H), 5.12 (d, J=11.7, 1H), 5.02 (s, 1H), 4.32 (d, J=11.4, 2H), 4.26 (d, J=11.4, 2H), 3.11 (m, 1H), 2.09 (s, 6H), 2.01 (s, 3H), 2.00-1.94 (m, 2H), 1.88-1.72 (m, 2H).

### Step J: Preparation of (1S)-3-[(2S,3R)-2-[5-{4-(acetyloxy)-3-[(acetyloxy)methyl]-3-hydroxybutyl}-2-(benzyloxy)phenyl]-1-(4-{2-[4-(aminocarbonyl)-1-3-thiazol-2-yl]ethyl}phenyl)-4-oxoazetidin-3-yl]-1-(4-fluorophenyl)propyl acetate

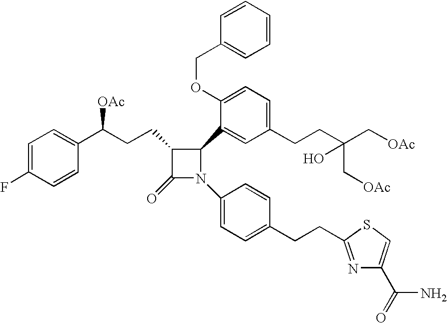

The title compound was prepared from (1S)-3-[(2S,3R)-2-[5-{4-(acetyloxy)-3-[(acetyloxy)methyl]-3-hydroxybut-1-yn-1-yl}-2-(benzyloxy)phenyl]-1-(4-{[4-(aminocarbonyl)-1-3-thiazol-2-yl]ethylnyl}phenyl)-4-oxoazetidin-3-yl]-1-(4-fluorophenyl)propyl acetate (intermediate from step I), according to the procedure of Example 2, step E. ¹HNMR (500 MHz, CDCl₃) δ: 8.01 (s, 1H), 7.44-7.39 (m, 5H), 7.20 (d, J=8.2, 2H), 7.16-7.07 (m, 6H), 6.98-6.95 (m, 4H), 5.72 (br s, 1H), 5.58 (t, J=6.4, 1H), 5.14 (d, J=11.6, 1H), 5.09 (d, J=11.6, 1H), 5.05 (d, J=1.6, 1H), 4.06 (m, 4H), 3.27 (t, J=7.3, 2H), 3.08-3.04 (m, 3H), 2.62-2.56 (m, 2H), 2.09 (s, 6H) 2.04-1.96 (m, 5H), 1.88-1.67 (m, 5H).

### Step K: Preparation of 2-[2-(4-{(2S,3R)-2-{2-(benzyloxy)-5-[3,4-dihydroxy-3-(hydroxymethyl)butyl]phenyl}-3-[(3S)-3-(4-fluorophenyl)-3-hydroxypropyl]-4-oxoazetidin-1-yl}phenyl)ethyl]-1,3-thiazole-4-carboxamide

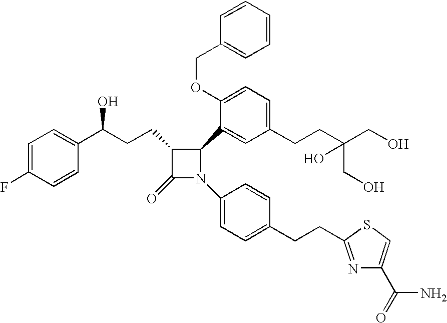

The title compound was prepared from (1S)-3-[(2S,3R)-2-[5-{4-(acetyloxy)-3-[(acetyloxy)methyl]-3-hydroxybutyl}-2-(benzyloxy)phenyl]-1-(4-{2-[4-(aminocarbonyl)-1-3-thiazol-2-yl]ethyl}phenyl)-4-oxoazetidin-3-yl]-1-(4-fluorophenyl)propyl acetate (intermediate from step J), according to the procedure of Example 2, step F. ¹HNMR (500 MHz, DMSO-d6) δ: 8.05 (s, 1H), 7.63 (br s, 1H), 7.51 (br s, 1H) 7.40 (d, J=7.1, 2H), 7.36-7.32 (m, 3H), 7.20 (dd, J=8.5 and 6.0, 2H), 7.16 (d, J=8.2, 2H), 7.08-7.04 (m, 6H), 7.00 (s, 1H), 5.21 (d, J=4.6, 1H), 5.15 (d, J=12.1, 1H), 5.10 (d, J=12.1, 1H), 5.03 (d, J=1.6, 1H), 4.42 (m, 1H), 4.35 (t, J=5.7, 2H), 4.01 (s, 1H), 3.24 (m, 6H), 3.14 (m, 1H), 2.97 (t, J=7.8, 2H), 2.46 (m, 1H), 1.80 (m, 1H), 1.76-1.64 (m, 3H), 1.50-1.46 (m, 2H).

### Step L: Preparation of 2-[2-(4-{(2S,3R)-2-{5-[3,4-dihydroxy-3-(hydroxymethyl)butyl]-2-hydroxyphenyl}-3-[(3S)-3-(4-fluorophenyl)-3-hydroxypropyl]-4-oxoazetidin-1-yl}phenyl)ethyl]-1,3-thiazole-4-carboxamide

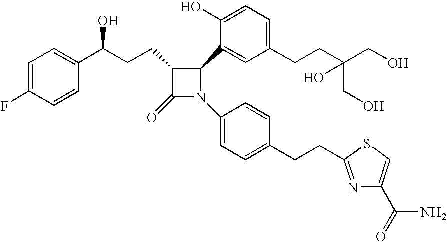

10% Palladium on carbon (20 mg) was added to a nitrogen flushed solution of 2-[2-(4-{(2S,3R)-2-{2-(benzyloxy)-5-[3,4-dihydroxy-3-(hydroxymethyl)butyl]phenyl}-3-[(3S)-3-(4-fluorophenyl)-3-hydroxypropyl]-4-oxoazetidin-1-yl}phenyl)ethyl]-1,3-thiazole-4-carboxamide (intermediate from step K) (80 mg, 0.106 mmol) in ethanol (5 ml), and the resulting mixture stirred under an atmosphere of hydrogen for 72 hours. The catalyst was removed by filtration through filter aid and the solvent removed under vacuum. The residue was purified by prep HPLC (C18 Sunfire column) eluting with gradient CH₃CN/0.1% aq. TFA (10 to 60%) and the appropriate fractions freeze dried to afford the title compound. m/z (ES) 646 (100%) (M-OH)⁺. ¹HNMR (500 MHz, DMSO-d6) δ: 9.56 (s, 1H), 8.05 (s, 1H), 7.63 (br s, 1H), 7.50 (br s, 1H) 7.28 (dd, J=8.2 and 5.7, 2H), 7.17 (m, 2H), 7.12-7.08 (m, 4H), 6.95 (s, 1H), 6.91 (d, J=8.2, 1H), 6.74 (d, J=8.3, 1H), 4.95 (s, 1H), 4.47 (t, J=6.0, 1H), 3.24 (m, 5H), 3.15 (m, 1H), 2.97 (t, J=7.8, 2H), 2.43 (m, 2H), 1.83 (m, 1H), 1.79-1.70 (m, 3H), 1.46 (m, 2H).

### Example 50

### Preparation of (3R,4S)-4-{4-[3,4-dihydroxy-3-(hydroxymethyl)butyl]-2-hydroxyphenyl}-3-[(3S)-3-(4-fluorophenyl)-3-hydroxypropyl]-1-{4-[2-(1H-1,2,4-triazol-3-yl)ethyl]phenyl}azetidin-2-one

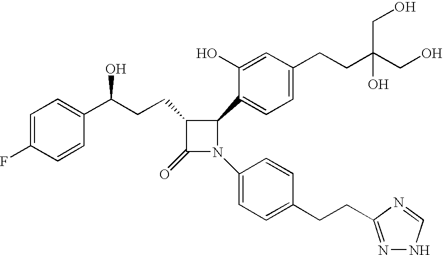

Using procedures outlined in Scheme I and specifically demonstrated in Example 1, steps D-H and Example 49, steps K and L, the title compound may be prepared from 4-(2S,3R)-3-[(3S)-3-(acetyloxy)-3-(4-fluorophenyl)propyl]-2-[2-(benzyloxy)-4-iodophenyl]-4-oxoazetidin-1-yl}phenyl acetate (i-48), and intermediate 3-ethynyl-1-trityl-1H-1,2,4-triazole (i-10).

### Example 51

### Step A: Preparation of 4-[(2S,3R)-3-[3-[(3S)-3-(4-fluorophenyl)-3-hydroxypropyl]-2-(4-iodophenyl)-4-oxoazetidin-1-yl]phenyl trifluoromethanesulfonate

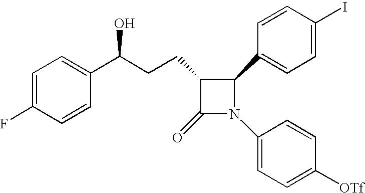

To a solution of (3R,4S)-3-[(3S)-3-(4-fluorophenyl)-3-hydroxypropyl]-1-(4-hydroxyphenyl)-4-(4-iodophenyl)azetidin-2-one (intermediate from Example 1, step C) (26.6 g, 47.6 mmol), and pyridine (4.6 ml, 57.1 mmol) in anhydrous CH₂Cl₂ (300 ml) cooled at 0° C. was added slowly trifluoromethane sulfonic anhydride (8.8 ml, 52.3 mmol). The resulting mixture was stirred at 0° C. for 2 hours, then washed with water (500 ml), sat. NaCl (100 ml), dried over MgSO₄, filtered and evaporated. The residue was purified by MPLC on silica gel eluting with a gradient rising from 100% hexanes to 60% EtOAc in hexanes to afford the title compound. ¹HNMR (500 MHz, CDCl₃) δ: 7.74 (d, J=8.2, 2H), 7.32-7.29 (m, 4H), 7.17 (d, J=9.2, 2H), 7.10 (d, J=8.5, 2H), 7.03 (t, J=8.7, 2H), 4.72 (m, 1H), 4.62 (d, J=2.5, 1H), 3.13 (m, 1H), 2.33 (d, J=2.6, 1H), 2.05-1.88 (m, 4H).

### Step B: Preparation of 2-[(acetyloxy)methyl]-4-{4-[(2S,3R)-3-[(3S)-3-(4-fluorophenyl)-3-hydroxypropyl]-4-oxo-1-(4-{[(trifluoromethyl)sulfonyl]oxy}phenyl)azetidin-2-yl]phenyl}-2-hydroxybut-3-yn-1-yl acetate

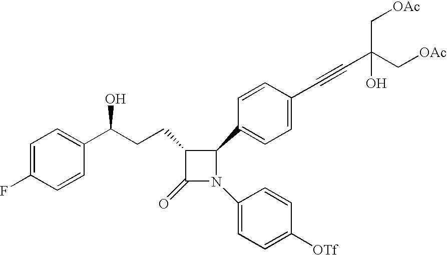

The title compound was prepared from 4-[(2S,3R)-3-[3-[(3S)-3-(4-fluorophenyl)-3-hydroxypropyl]-2-(4-iodophenyl)-4-oxoazetidin-1-yl]phenyl trifluoromethanesulfonate (intermediate from step A), and 2-ethynylpropane-1,2,3-triol 1,3-diacetate according to the procedure of Example 1, step F. ¹HNMR (500 MHz, CDCl₃) δ: 7.46 (d, J=8.3, 2H), 7.31-7.28 (m, 6H), 7.16 (d, J=9.1, 2H), 7.03 (t, J=8.7, 2H), 4.72 (m, 1H), 4.66 (d, J=2.3, 1H), 4.38 (d, J=11.5, 2H), 4.31 (d, J=11.5, 2H), 3.23 (s, 1H), 3.13 (m, 1H), 2.42 (d, J=3.0, 1H), 2.15 (s, 6H), 2.05-1.86 (m, 4H).

### Step C: Preparation of 2-[(acetyloxy)methyl]-4-{4-[(2S,3R)-3-[3-(4-fluorophenyl)-3-oxopropyl]-4-oxo-1-(4-{[(trifluoromethyl)sulfonyl]oxy}phenyl)azetidin-2-yl]phenyl}-2-hydroxybut-3-yn-1-yl acetate

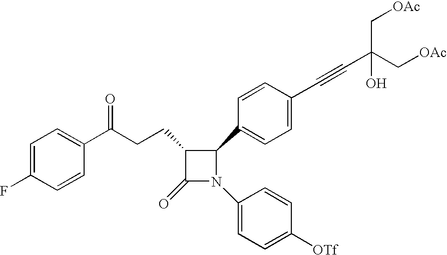

To a solution of 2-[(acetyloxy)methyl]-4-{4-[(2S,3R)-3-[(3S)-3-(4-fluorophenyl)-3 hydroxypropyl]-4-oxo-1-(4-{[(trifluoromethyl)sulfonyl]oxy}phenyl)azetidin-2-yl]phenyl}-2-hydroxybut-3-yn-1-yl acetate (intermediate from step B), (960 mg, 1.33 mmol) in anhydrous CH₂Cl₂ (20 ml) was added Dess-Martin periodinane (734 mg, 1.73 mmol), and the resulting mixture stirred at room temperature for 2 hours. The mixture was washed with sat. NaHCO₃, sat. NaCl, dried over Na₂SO₄, filtered and evaporated. The residue was purified by MPLC on silica gel eluting with a gradient rising from 100% hexanes to 45% EtOAc in hexanes to give the title compound. ¹HNMR (500 MHz, CDCl₃) δ: 8.01 (m, 2H), 7.46 (d, J=8.3, 2H), 7.33 (m, 4H), 7.19-7.14 (m, 4H), 4.80 (d, J=2.6, 1H), 4.38 (d, J=11.2, 2H), 4.31 (d, J=11.2, 2H), 3.34-3.28 (m, 1H), 3.25-3.16 (m, 2H), 3.04 (s, 1H), 2.48-2.43 (m, 1H), 2.35-2.28 (m, 1H), 2.16 (s, 6H).

### Step D: Preparation of (3R,4S)-4-{4-[3,4-dihydroxy-3-(hydroxymethyl)butyl]phenyl}-3-[3-(4-fluorophenyl)-3-oxopropyl]-1-{4-[2-(1H-1,2,4-triazol-3-yl)ethyl]phenyl}azetidin-2-one

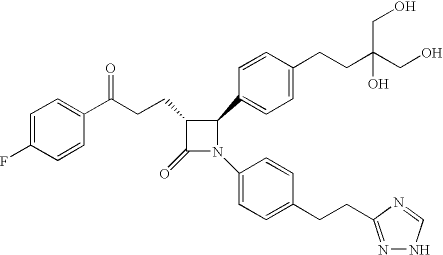

The title compound was prepared from 2-[(acetyloxy)methyl]-4-{4-[(2S,3R)-3-[3-(4-fluorophenyl)-3-oxopropyl]-4-oxo-1-(4-{[(trifluoromethyl)sulfonyl]oxy}phenyl)azetidin-2-yl]phenyl}-2-hydroxybut-3-yn-1-yl acetate (intermediate from step C), according to the general procedures outlined in Example 2, steps A-F. m/z (ES) 587 (100%) (M+H)⁺. ¹HNMR (500 MHz, DMSO-d6) δ: 7.99 (m, 3H), 7.33-7.27 (m, 4H), 7.15 (d, J=8.0, 2H), 7.09 (m, 4H), 4.93 (d, J=1.6, 1H), 3.27 (m, 4H), 3.21-3.18 (m, 2H), 3.12-3.09 (m, 1H), 2.86 (m, 4H), 2.58-2.55 (m, 2H), 2.10 (q, J=7.3, 2H), 1.58-1.54 (m, 2H).

### Example 52

### Preparation of 3-[2-(4-{(2S,3R)-2-{4-[3,4-dihydroxy-3-(hydroxymethyl)butyl]phenyl}-3-[(3S)-3-(4-fluorophenyl)-3-hydroxpropyl]-4-oxoazetidin-1-yl}phenyl)ethyl]-1H-1,2,4-triazole-5-carboxamide

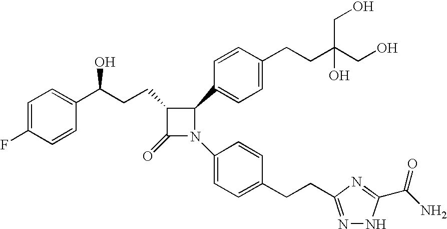

Using procedures outlined in Scheme II and described in Example 2, the title compound may be prepared from (1S)-3-[(2S,3R)-2-(4-{4-(acetyloxy)-3-[(acetyloxy)methyl]-3-hydroxybut-1-yn-1-yl}phenyl)-1-(4-ethynylphenyl)-4-oxoazetidin-3-yl]-1-(4-fluorophenyl)propyl acetate (intermediate from Example 2, step B), and intermediate 3-iodo-1-trityl-1,2,4-triazole-5-carboxamide (i-53).

### Example 53

### Preparation of 342-(4-{(2S,3R)-2-{-4-[3,4-dihydroxy-3-(hydroxymethyl)butyl]phenyl}-3-[(3S)-3-(4-fluorophenyl)-3-hydroxpropyl]-4-oxoazetidin-1-yl}phenyl)ethyl]-1H-1,2,4-triazole-5-carbonitrile

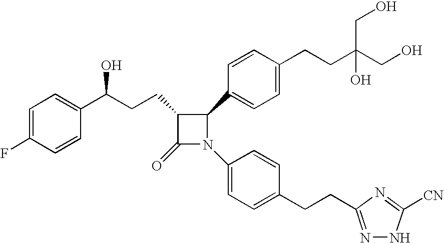

Using procedures outlined in scheme II and described in Example 2, the title compound may be prepared from (1S)-3-[(2S,3R)-2-(4-{4-(acetyloxy)-3-[(acetyloxy)methyl]-3-hydroxybut-1-yn-1-yl}phenyl)-1-(4-ethynylphenyl)-4-oxoazetidin-3-yl]-1-(4-fluorophenyl)propyl acetate (intermediate from Example 2, step B), and intermediate 5-cyano-3-iodo-1-trityl-1,2,4-triazole (i-54).

### Example 54

### Preparation of (3R,4S)-4-{-4-[3,4-dihydroxy-3-(hydroxymethyl)butyl]phenyl}-3-[(3S)-3-(4-fluorophenyl)-3-hydroxypropyl]-1-(4-{2-[5-(hydroxymethyl)-1H-1,2,4-triazol-3-yl]ethyl}phenyl)azetidin-2-one

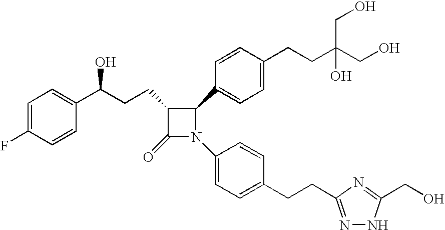

Using procedures outlined in scheme II and described in Example 2, the title compound may be prepared from (1S)-3-[(2S,3R)-2-(4-{4-(acetyloxy)-3-[(acetyloxy)methyl]-3-hydroxybut-1-yn-1-yl}phenyl)-1-(4-ethynylphenyl)-4-oxoazetidin-3-yl]-1-(4-fluorophenyl)propyl acetate (intermediate from Example 2, step B), and intermediate 3-iodo-1-trityl-1,2,4-triazole-5-methanol (i-55).

While the invention has been described and illustrated with reference to certain particular embodiments thereof, those skilled in the art will appreciate that various changes, modifications and substitutions can be made therein without departing from the spirit and scope of the invention. It is intended, therefore, that the invention be defined by the scope of the claims which follow and that such claims be interpreted as broadly as is reasonable.

What is claimed is:

1. A compound of structural Formula I 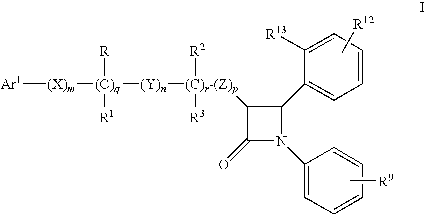
and the pharmaceutically acceptable salts thereof, wherein
Ar¹ is selected from the group consisting of aryl and R⁴-substituted aryl;
X, Y and Z are independently selected from the group consisting of —CH₂—, —CH(C₁₋₆alkyl)- and —C(C₁₋₆alkyl)₂-;
R is selected from the group consisting of —OR⁶, —O(CO)R⁶, —O(CO)OR⁸, —O(CO)NR⁶R⁷, a sugar residue, a disugar residue, a trisugar residue and a tetrasugar residue;
R¹ is selected from the group consisting of —H, —C₁₋₆alkyl and aryl, or R and R¹ together are oxo;
R² is selected from the group consisting of —OR⁶, —O(CO)R⁶, —O(CO)OR⁸ and —O(CO)NR⁶R⁷;
R³ is selected from the group consisting of —H, —C₁₋₆alkyl and aryl, or R² and R³ together are oxo;
q and r are integers each independently selected from 0 and 1 provided that at least one of q and r is 1;
m, n and p are integers each independently selected from 0, 1, 2, 3 and 4, provided that the sum of m, n, p, q and r is 1, 2, 3, 4, 5 or 6;
t is an integer selected from 0, 1 and 2;
R⁴ is 1-5 substituents independently selected at each occurrence from the group consisting of: —OR⁵, —O(CO)R⁵, —O(CO)OR⁸, —O(CO)NR⁵R⁶, —NR⁵R⁶, —NR⁵(CO)R⁶, —NR⁵(CO)OR⁸, —NR⁵(CO)NR⁶R⁷, —NR⁵SO₂R⁸, —COOR⁵, —CONR⁵R⁶, —COR⁵, —SO₂NR⁵R⁶, —S(O)ₜR⁸, —O—C₁₋₁₀alkyl-COORS, —O—C₁₋₁₀alkyl-CONR⁵R⁶ and fluoro;
R⁵, R⁶ and R⁷ are independently selected at each occurrence from the group consisting of —H, —C₁₋₆alkyl, aryl and aryl-substituted-C₁₋₆alkyl;
R⁸ is selected from the group consisting of —C₁₋₆alkyl, aryl and aryl-substituted —C₁₋₆alkyl;
R⁹ is selected from the group consisting of —C₁₋₈alkyl-Hetcy, —(CH₂)₀₋₂CH═CH—C₀₋₆alkyl-Hetcy, —C≡C—C₀₋₆alkyl-Hetcy and —C₁₋₈alkyl-NH-Hetcy;
Hetcy is selected from the group consisting of:
(a) a 5-membered aromatic or partially unsaturated heterocyclic ring containing 1 to 4 heteroatoms selected from 1 to 4 of N, zero to 1 of S, and zero to 1 of O, wherein the heterocyclic ring is optionally mono- or di-substituted with R¹⁴,
(b) a 6-membered aromatic heterocyclic ring containing 1 to 3 N heteroatoms, wherein the heterocyclic ring is optionally mono- or di-substituted with R¹⁴, and
(c) a 6-membered saturated heterocyclic ring containing 1 to 3 heteroatoms selected from 1-3 of N, zero to 1 of O, and zero to 1 of S(O)ₜ, and wherein the heterocyclic ring is optionally mono- or di-substituted with R¹⁴;
R^{10a} is —C₁₋₃alkyl optionally substituted with one or more substituents selected from the group consisting of —OH, phenyl and 1-3 of fluoro;
R¹⁰ is selected from the group consisting of —H and —C₁₋₃alkyl optionally substituted with one or more substituents selected from the group consisting of —OH, phenyl and 1-3 of fluoro;
R¹¹ is selected from the group consisting of —H and —C₁₋₃alkyl optionally substituted with one or more substituents selected from the group consisting of —OH, phenyl and 1-3 of fluoro;
R¹² is selected from the group consisting of —C₁₋₁₅alkyl mono- or poly-substituted with —OH, —C₂₋₁₅alkenyl mono- or poly-substituted with —OH, —C₂₋₁₅alkynyl mono- or poly-substituted with —OH, and —C₁₋₃alkyl-C₃₋₆cycloalkyl wherein each carbon in the cycloalkyl ring is optionally substituted with —OH;
R¹³ is selected from the group consisting of —H and —OH; and
R¹⁴ is independently selected at each occurrence from the group consisting of: R^{10a}, —C₁₋₃ alkyl-COOR¹⁰, —C₁₋₃alkyl-C(O)NR¹⁰R¹¹, —C₁₋₃alkyl-SO₂—R^{10a}, —C₁₋₃alkyl-O—R^{10a}, —COOR¹⁰, —OC(O)—R^{10a}, —C(O)NR¹⁰R¹¹, —NR¹⁰R¹¹, —CN, —OH and oxo.

2. The compound of claim 1 wherein R⁹ is selected from the group consisting of —C₁₋₈alkyl-Hetcy, —(CH₂)₀₋₂CH═CH—C₁₋₆alkyl-Hetcy, —C≡C—C₁₋₆alkyl-Hetcy and —C₁₋₈alkyl-NH-Hetcy and R¹⁴ is independently selected at each occurrence from the group consisting of R^{10a}, —C₁₋₃alkyl-COOR¹⁰, —C₁₋₃alkyl-C(O)NR¹⁰R¹¹, —C₁₋₃ alkyl-SO₂—R^{10a}, —C₁₋₃alkyl-O—R^{10a}, —COOR¹⁰, —OC(O)—R^{10a}, —C(O)NR¹⁰R¹¹, —NR¹⁰R¹¹, —OH and oxo.

3. The compound of claim 1 wherein R⁹ is —C₁₋₈alkyl-Hetcy and R¹² is —C₁₋₈alkyl mono- or poly-substituted with —OH.

4. The compound of claim 3 wherein R⁹ is —C₂₋₃ n-alkyl-Hetcy and R¹² is —C₃₋₈ alkyl mono- or poly-substituted with —OH.

5. The compound of claim 3 wherein R¹² is —(CH₂)₂₋₃—C(OH)(CH₂OH)₂.

6. The compound of claim 1 wherein r is zero, m is zero, q is 1, n is 1 and p is 1.

7. The compound of claim 6 having structural Formula Ia 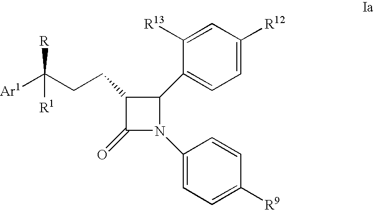
and the pharmaceutically acceptable salts thereof.

8. The compound of claim 7 wherein R⁹ is —C₁₋₈alkyl-Hetcy and R¹² is —C₁₋₈alkyl mono- or poly-substituted with —OH.

9. The compound of claim 8 wherein R⁹ is —C₂₋₃ n-alkyl-Hetcy and R¹² is —C₃₋₈ alkyl mono- or poly-substituted with —OH.

10. The compound of claim 8 wherein R¹² is —(CH₂)₂₋₃—C(OH)(CH₂OH)₂.

11. The compound of claim 7 having structural Formula Ib 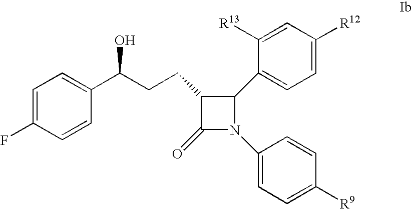
and the pharmaceutically acceptable salts thereof.

12. The compound of claim 11 wherein R⁹ is —C₁₋₈alkyl-Hetcy and R¹² is —C₁₋₈alkyl mono- or poly-substituted with —OH.

13. The compound of claim 12 wherein R⁹ is —C₂₋₃ n-alkyl-Hetcy and R¹² is —C₃₋₈ alkyl mono- or poly-substituted with —OH.

14. The compound of claim 12 wherein R¹² is —(CH₂)₂₋₃—C(OH)(CF₂OH)₂.

15. The compound of claim 1 selected from the group consisting of:
(3R,4S)-4-{4-[3,4-dihydroxy-3-(hydroxymethyl)butyl]phenyl}-3-[(3S)-3-(4-fluorophenyl)-3-hydroxypropyl]-1-{4-[3-(1H-1,2,4-triazol-1-yl)propyl]phenyl}azetidin-2-one;
(3R,4S)-4-{4-[3,4-dihydroxy-3-(hydroxymethyl)butyl]phenyl}-3-[(3S)-3-(4-fluorophenyl)-3-hydroxypropyl]-1-{4-[2-(1H-1,2,4-triazol-5-yl)ethyl]phenyl}azetidin-2-one;
(3R,4S)-4-{4-[3,4-dihydroxy-3-(hydroxymethyl)butyl]phenyl}-3-[(3S)-3-(4-fluorophenyl)-3-hydroxypropyl]-1-{4-[3-(1,3-thiazol-2-ylamino)propyl]phenyl}azetidin-2-one;
(3R,4S)-3-[(3S)-3-(4-fluorophenyl)-3-hydroxypropyl]-4-{4-[2,3,4,5-tetrahydroxy-4-(hydroxymethyl)pentyl]phenyl}-1-{4-[2-(1H-1,2,4-triazol-3-yl)ethyl]phenyl}azetidin-2-one;
(3R,4S)-3-[(3S)-3-(4-fluorophenyl)-3-hydroxypropyl]-4-{4-[1, 2,3,4-tetrahydroxy-3-(hydroxymethyl)butyl]phenyl}-1-{4-[2-(1H-1,2,4-triazol-3-yl)ethyl]phenyl}azetidin-2-one;
(3R,4S)-4-{4-[4,5-dihydroxy-4-(hydroxymethyl)pentyl]phenyl}-3-[(3S)-3-(4-fluorophenyl)-3-hydroxpropyl]-1-{4-[2-(1H-1,2,4-triazol-3-yl)ethyl]phenyl)azetidin-2-one;
(3R,4S)-3-[(3S)-3-(4-fluorophenyl)-3-hydroxypropyl]-4-{4-[3,4,5,6-tetrahydroy-3,5-bis(hydroxymethyl)hexyl]phenyl}-1-{4-[2-(1H-1,2,4-triazol-3-yl)ethyl]phenyl}azetidin-2-one;
(3R,4S)-3-[(3S)-3-(4-fluorophenyl)-3-hydroxypropyl]-4-{4-[3,4,5,6-tetrahydroy-3,5-bis(hydroxymethyl)hexyl]phenyl}-1-{4-[2-(1H-1,2,4-triazol-3-yl)ethyl]phenyl}azetidin-2-one;
2-[2-(4-{(2S,3R)-2-{5-[3,4-dihydroxy-3-(hydroxymethyl)butyl]-2-hydroxyphenyl}-3-[(3S)-3-(4-fluorophenyl)-3-hydroxypropyl]-4-oxoazetidin-1-yl}phenyl)ethyl]-1,3-thiazole-4-carboxamide;
(3R,4S)-4-{4-[3,4-dihydroxy-3-(hydroxymethyl)butyl]-2-hydroxyphenyl}-3-[(3S)-3-(4-fluorophenyl)-3-hydroxypropyl]-1-{4-[2-(1H-1,2,4-triazol-3-yl)ethyl]phenyl}azetidin-2-one;
(3R,4S)-4-{4-[3,4-dihydroxy-3-(hydroxymethyl)butyl]phenyl}-3-[3-(4-fluorophenyl)-3-oxopropyl]-1-{4-[2-(1H-1,2,4-triazol-3-yl)ethyl]phenyl}azetidin-2-one;
3-[2-(4-{(2S,3R)-2-{4-[3,4-dihydroxy-3-(hydroxymethyl)butyl]phenyl}-3-[(3S)-3-(4-fluorophenyl)-3-hydroxpropyl]-4-oxoazetidin-1-yl}phenyl)ethyl]-1H-1,2,4-triazole-5-carboxamide;
3-[2-(4-{(2S,3R)-2-{-4-[3,4-dihydroxy-3-(hydroxymethyl)butyl]phenyl}-3-[(3S)-3-(4-fluorophenyl)-3-hydroxpropyl]-4-oxoazetidin-1-yl}phenyl)ethyl]-1H-1,2,4-triazole-5-carbonitrile;
(3R,4S)-4-{4-[3,4-dihydroxy-3-(hydroxymethyl)butyl]phenyl}-3-[(3S)-3-(4-fluorophenyl)-3-hydroxypropyl]-1-(4-{2-[5-(hydroxymethyl)-1H-1,2,4-triazol-3-yl]ethyl}phenyl)azetidin-2-one;
and the pharmaceutically acceptable salts thereof.

16. The compound of claim 1 having the following structural formula 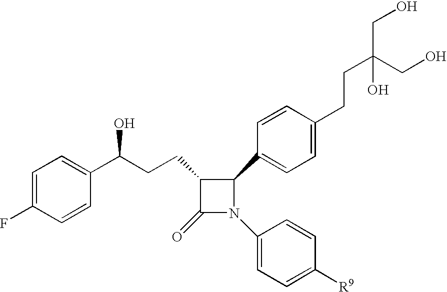
selected from the group consisting of:

| Compound | R⁹ |
|---|---|
| 4 | 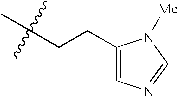 |
| 5 | 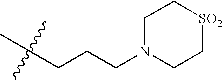 |
| 6 | 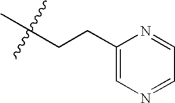 |
| 7 | 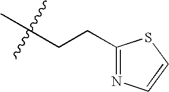 |
| 8 | 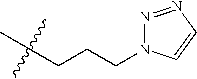 |
| 9 | 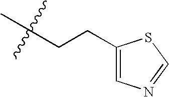 |
| 10 | 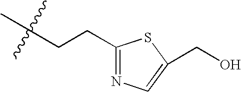 |
| 11 | 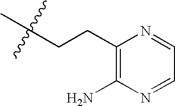 |
| 12 | 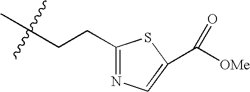 |
| 13 | 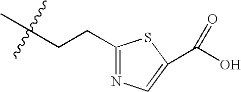 |
| 14 | 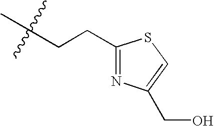 |
| 15 | 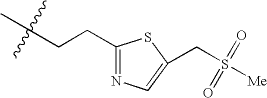 |
| 16 | 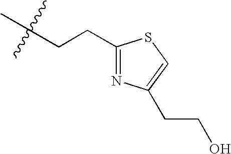 |
| 17 | 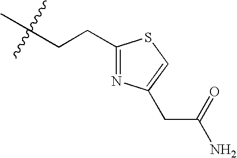 |
| 18 | 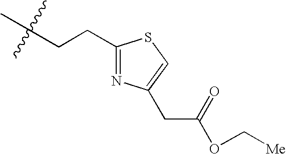 |
| 19 | 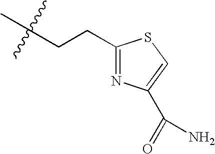 |
| 20 | 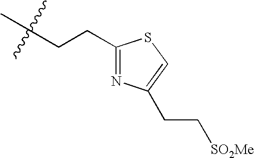 |
| 21 | 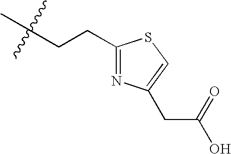 |
| 22 | 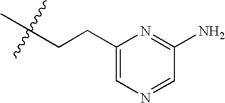 |
| 23 | 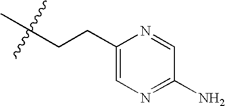 |
| 24 | 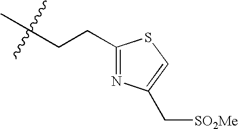 |
| 25 | 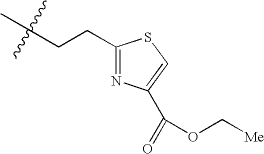 |
| 26 | 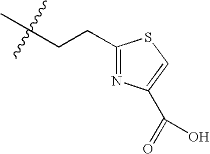 |
| 27 | 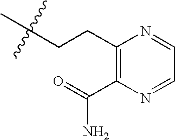 |
| 29 | 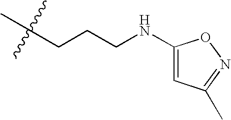 |
| 30 | 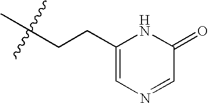 |
| 31 | 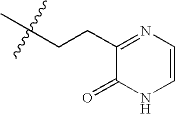 |
| 32 | 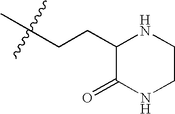 |
| 33 | 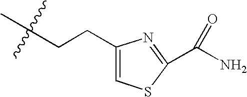 |
| 34 | 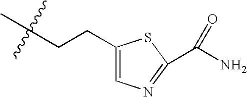 |
| 35 | 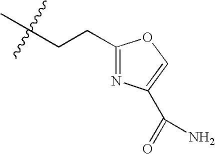 |
| 36 | 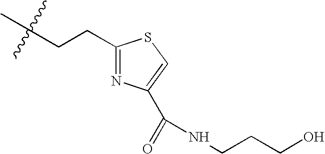 |
| 37 | 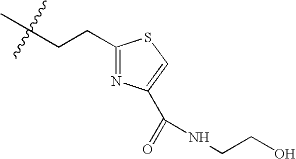 |
| 38 | 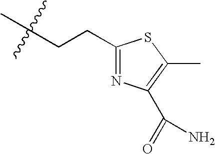 |
| 39 | 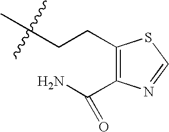 |
| 40 | 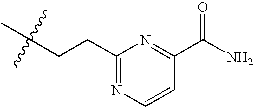 |
| 41 | 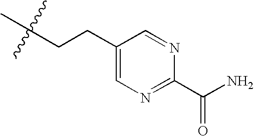 |
| 42 | 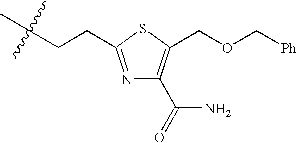 |
| 43 | 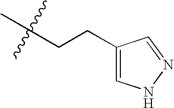 |

and the pharmaceutically acceptable salts thereof.

17. A method for lowering plasma LDL-cholesterol levels comprising administering a therapeutically effective amount of a compound of claim 1 to a patient in need of such treatment

18. The method of claim 17 comprising administering to a patient in need of such treatment a therapeutically effective amount of a compound of claim 1 in combination with a therapeutically effective amount of at least one additional active agent selected from a lipid modifying agent, an anti-diabetic agent and an anti-obesity agent.

19. A method for reducing the risk for having an atherosclerotic disease event comprising administering a prophylactically effective amount of a compound of claim 1 to a patient at risk for such an event.

20. A pharmaceutical composition comprising a compound of claim 1 and a pharmaceutically acceptable carrier, and optionally comprising at least one additional active agent selected from a lipid modifying agent, an anti-diabetic agent and an anti-obesity agent.
